(19) United States
(12) Patent Application Publication
(10) Pub. No.: US 2025/0242012 A1
(43) Pub. Date: Jul. 31, 2025

(54) GENE DELIVERY SYSTEM

(71) Applicant: Symvivo Corporation, Burnaby (CA)

(72) Inventors: Herbert Alexander Graves, Burnaby (CA); Sheetal Raithatha, Burnaby (CA); Eric Sievers, La Jolla, CA (US); Umesh Ramachandran, Burnaby (CA); Kevin Zhang, Burnaby (CA); Elena Topchiy, Burnaby (CA); Navi Gill, Burnaby (CA); Sally Sayer, Burnaby (CA); Fenil Shah, Burnaby (CA); Catherine Steer, Burnaby (CA); Ryan Thomas, Burnaby (CA); Maye Cheng, Burnaby (CA); Sharanya Sivaraman, Burnaby (CA); Lucy Lu, Burnaby (CA); Sophie Yurist-Doutsch, Burnaby (CA); Oleg Tyurin, Burnaby (CA); Lloyd Mackenzie, Burnaby (CA)

(73) Assignee: Symvivo Corporation, Burnaby, BC (CA)

(21) Appl. No.: 17/802,118

(22) PCT Filed: Feb. 25, 2021
(86) PCT No.: PCT/CA2021/050236

§ 371 Date: Aug. 24, 2022

Related U.S. Application Data
(60) Provisional application No. 62/981,464, filed on Feb. 25, 2020.
(60) Provisional application No. 63/079,841, filed on Sep. 17, 2020.

(51) Int. Cl. A61K 39/215; A61K 9/00; A61K 39/00; A61K 45/06; A61P 31/14; C07K 14/195

(52) U.S. Cl. CPC A61K 39/215; A61K 9/0053; A61K 45/06; A61P 31/14; C07K 14/195; A61K 2039/523; A61K 2039/542; A61K 2039/545; A61K 2039/572; C07K 2319/10; C07K 2319/80; C12N 2770/20022; C12N 2770/20034

(57) ABSTRACT

A system for delivering a payload nucleic acid into target cells of a subject and production of a payload (or payloads) encoded by the payload nucleic acid in the cells. The system includes a Bifidobacterium sp. bacterium comprising a plasmid and a transporter nucleic acid, the transporter nucleic acid configured for expression in the bacterium. The transporter nucleic acid encodes a transporter polypeptide comprising, in an amino-terminal to carboxy-terminal order, a bacterial secretion signal peptide, a DNA-binding domain to bind the plasmid, and a cell penetrating peptide. The transporter polypeptide complexes with the plasmid and transports the plasmid from the bacterium into the target cells. The plasmid encodes one or more payloads (protein and/or ribonucleic acid) for production in the target cells. The target cells may be colonic cells. When the payload(s) include an antigen, the system may be a DNA vaccine.

### FIELD OF INVENTION

The present invention relates to gene delivery systems. In particular, the present invention relates to bacteria that colonize a subject and deliver a plasmid to the subject's cells for expression of gene payload(s).

### BACKGROUND OF THE INVENTION

The recent emergence of a novel, highly infectious coronavirus, SARS-CoV-2, has led to the rapid progression of COVID-19 into a menacing global pandemic. There is therefore a need for new vaccines, including, e.g., vaccines against COVID-19.

More generally, there exists a need for gene delivery systems that effectively transport gene payloads into cells of a subject, e.g. for therapeutic or immunoprotective purposes. One gene delivery system is described in PCT Publication Nos. WO/2015/120541 and WO/2015/120542, which disclosed transformation of Bifidobacterium longum cells with a plasmid capable of expressing a green fluorescent protein (GFP) marker and a Novel Hybrid Protein, referred to herein as a “transporter polypeptide” or “Hybrid Transport Protein” (HTP). The transporter polypeptide comprised a DNA-binding domain, a bacterial secretion signal peptide and a cell penetrating peptide (CPP) domain. It was shown that expression of the transporter polypeptide in B. longum caused secretion of the transporter polypeptide and the plasmid encoding the transporter polypeptide from B. longum. It was also shown that complexes of transporter polypeptide and plasmid DNA were capable of transfecting mammalian cells in vitro to intracellularly express GFP encoded by the plasmid. Transporter polypeptides may therefore be useful for transporting plasmids from bacteria to eukaryotic target cells (including mammalian cells), which then express a payload in the target cell.

### SUMMARY

Prior to this application, it was unknown if gene delivery systems such as the transporter polypeptides in PCT Publication Nos. WO/2015/120541 and WO/2015/120542 could be adapted to produce vaccines. It was also unknown if such systems could be adapted for oral administration (or other forms of non-intravenous administration) to a subject to deliver genes for expression in the colon.

Various embodiments of this disclosure relate to a system for use in delivery of a payload nucleic acid into colonic cells (e.g. colonic epithelial cells, colonic immune cells, and/or cells of the lamina propria) of a subject and production of a payload encoded by the payload nucleic acid in the colonic cells (e.g. colonic epithelial cells, colonic immune cells, and/or colonic immune cells); a Bifidobacterium sp. bacterium comprising a plasmid and a transporter nucleic acid the transporter nucleic acid in operative association with a first promoter and a first terminator configured to express the transporter nucleic acid in the bacterium; the transporter nucleic acid having a sequence encoding a transporter polypeptide comprising, in an amino-terminal to carboxy-terminal order, a bacterial secretion signal peptide, a DNA-binding domain, and a cell penetrating peptide, the DNA-binding domain configured for association with the plasmid to form a polypeptide-plasmid complex, the bacterial secretion signal peptide configured for secretion of the polypeptide-plasmid complex from the bacterium, and the cell penetrating peptide configured for importing the polypeptide-plasmid complex into a colonic cell (e.g. a colonic epithelial cell, a colonic immune cell, or a cell of the lamina propria) of the subject; and the plasmid comprising a payload nucleic acid encoding a payload protein or a payload ribonucleic acid, the payload nucleic acid in operative association with a second promoter and a second terminator configured to express the payload nucleic acid in the colonic cell and produce the payload protein or the payload ribonucleic acid.

Various embodiments relate to a method for delivering a payload nucleic acid into colonic cells (e.g. colonic epithelial cells, colonic immune cells, and/or cells of the lamina propria) of a subject and causing the cells to produce a payload encoded by the payload nucleic acid, the method comprising administering to the subject a Bifidobacterium sp. bacterium comprising a plasmid and a transporter nucleic acid such that the bacterium colonizes the colon of the subject; the transporter nucleic acid is in operative association with a first promoter and a first terminator configured to express the transporter nucleic acid in the bacterium; the transporter nucleic acid encoding a transporter polypeptide comprising, in an amino-terminal to carboxy-terminal order, a bacterial secretion signal peptide, a DNA-binding domain, and a cell penetrating peptide, the DNA-binding domain configured for association with the plasmid to form a polypeptide-plasmid complex, the bacterial secretion signal peptide configured for secretion of the polypeptide-plasmid complex from the bacterium, and the cell penetrating peptide configured for importing the polypeptide-plasmid complex into a colonic cell (e.g. a colonic epithelial cell, a colonic immune cell, and/or a cell of the lamina propria) of the subject; and the plasmid comprising a payload nucleic acid having a sequence encoding a payload protein or a payload ribonucleic acid, the payload nucleic acid in operative association with a second promoter and a second terminator configured to express the payload nucleic acid in the colonic cell and produce the payload protein or the payload ribonucleic acid.

Various embodiments relate to a DNA vaccine comprising: a Bifidobacterium sp. bacterium comprising a plasmid and a transporter nucleic acid the transporter nucleic acid in operative association with a first promoter and a first terminator configured to express the transporter nucleic acid in the bacterium; the transporter nucleic acid encoding a transporter polypeptide comprising, in an amino-terminal to carboxy-terminal order, a bacterial secretion signal peptide, a DNA-binding domain, and a cell penetrating peptide, the DNA-binding domain configured for association with the plasmid to form a polypeptide-plasmid complex, the bacterial secretion signal peptide configured for secretion of the polypeptide-plasmid complex from the bacterium, and the cell penetrating peptide configured for importing the polypeptide-plasmid complex into a cell of a subject; and the plasmid comprising a payload nucleic acid encoding a payload protein, the payload nucleic acid in operative association with a second promoter and a second terminator configured to express the payload gene in the cell and produce the payload protein, wherein the payload protein is a component of a pathogen or wherein the payload protein is an antigen that is specific for or associated with a pathology (e.g. cancer, a toxin, or any other foreign or pathologically relevant molecule). In some embodiments, the DNA vaccine is a coronavirus vaccine, wherein the payload nucleic acid encodes one or more components of a coronavirus, e.g. spike, matrix (also called membrane glycoprotein), envelope and/or nucleocapsid, or fragment/derivative thereof). In some embodiments, the coronavirus is SARS-CoV-2.

Various embodiments relate to a method of vaccinating a subject against a pathogen, the method comprising administering to the subject a DNA vaccine defined herein, wherein the payload nucleic acid encodes one or more components of the pathogen.

Various embodiments relate to a method of vaccinating a subject against a coronavirus, the method comprising administering to the subject a DNA vaccine defined herein, wherein the payload nucleic acid encodes one or more components of a coronavirus (e.g. spike, matrix, envelope and/or nucleocapsid, or fragment/derivative thereof). In some embodiments, the coronavirus is a member of the betacoronavirus genus. In some embodiments, the coronavirus is SARS-CoV-2.

Various embodiments relate to a method of vaccinating a subject against a pathology, the method comprising administering to the subject the DNA vaccine defined herein, wherein the payload nucleic acid encodes an antigen that is specific for or associated with the pathology. Various embodiments relate to a method of vaccinating a subject against a cancer, the method comprising administering to the subject the DNA vaccine defined herein, wherein the payload nucleic acid encodes an antigen that is specific for or associated with the cancer.

### BRIEF DESCRIPTION OF THE DRAWINGS

These and other features of the invention will become more apparent from the following description in which reference is made to the appended drawings, as briefly described below.

FIG. 1 shows graphs of Gaussia luciferase fluorescence in blood drawn from bacTRL-treated mice on days 3, 6, 9 and 12.

FIG. 2 shows representative Gram-staining of colonies isolated from colons of bacTRL-treated mice. Panel A (M5). Panel B (M6). Panel C (M7). Panel D (M8).

FIG. 3 shows representative Gram-stained FFPE colon sections from bacTRL-treated mouse M9. Panel A—proximal region of colon (mid-to-late section). Panels B, C and D—10× and 100× magnified.

FIG. 4 shows representative Gram-stained FFPE colon sections from bacTRL-treated mouse M9. Panel A—beginning of middle region of colon. Panels B, C and D—100× magnified.

FIGS. 5A, 5B and 5C show representative immunofluorescent stained colon sections of bacTRL-treated mouse M14 at medial colon part (FIG. 5A), upper distal part (FIG. 5B) and lower distal part (FIG. 5C).

FIG. 6 shows Gram-stained colon sections of bacTRL-Spike treated mice. Panel A shows 4× objective magnification (arrow indicates a cluster of B. longum). Panel B shows 40× objective magnification of the B. longum cluster indicated in Panel A. Panel C shows 100× objective magnification of the B. longum cluster indicated in Panel A.

FIG. 7 shows colon sections of saline and bacTRL-Spike treated mice, respectively, stained with DAPI (nuclei; blue) and subjected to fluorescence microscopy to analyze spike protein expression and localization (green; magnification 10× objective).

FIG. 8, Panel A, shows a graph of % anti-Spike immunoreactivity against a commercially available recombinant trimeric spike ectodomain (S1+S2)in serially titrated serum samples collected from bacTRL-Spike-treated mice. FIG. 8, Panel B, shows a graph of anti-Spike serum binding titers to the SARS-CoV-2 ectodomain (S1+S2) in sera from bacTRL-Spike-treated mice.

FIG. 9, Panel A, shows a graph of % anti-Spike IgA binding activity against a commercially available recombinant trimeric spike ectodomain (S1+S2) in serially titrated fecal extracts collected from bacTRL-Spike-treated and saline-treated mice (day 21 post-immunization). FIG. 9, Panel B, shows a graph of fecal IgA antibody binding titers to the SARS-CoV-2 ectodomain (S1+S2) in the extracts from bacTRL-Spike-treated mice collected at day 21.

FIG. 10 shows a graph of % inhibition (% neutralizing antibody activity) in sera collected from saline and bacTRL-Spike treated mice, respectively, at days 21 and 40.

### DETAILED DESCRIPTION

### I. General Definitions

As used herein, the terms “comprising,” “having”, “including” and “containing,” and grammatical variations thereof, are inclusive or open-ended and do not exclude additional, unrecited elements and/or method steps. The term “consisting essentially of” when used herein in connection with a composition, use or method, denotes that additional elements and/or method steps may be present, but that these additions do not materially affect the manner in which the recited composition, method or use functions. The term “consisting of” (when used) herein in connection with a composition, use or method, excludes the presence of additional elements and/or method steps. A composition, use or method described herein as comprising certain elements and/or steps may also, in certain embodiments consist essentially of those elements and/or steps, and in other embodiments consist of those elements and/or steps, whether or not these embodiments are specifically referred to. A use or method described herein as comprising certain elements and/or steps may also, in certain embodiments consist essentially of those elements and/or steps, and in other embodiments consist of those elements and/or steps, whether or not these embodiments are specifically referred to.

A reference to an element preceded by the indefinite article “a” does not exclude the possibility that more than one of the elements is present, unless the context clearly requires that there be one and only one of the elements. The singular forms “a”, “an”, and “the” include plural referents unless the content clearly dictates otherwise. The use of the word “a” or “an” when used herein in conjunction with the term “comprising” may mean “one,” but it is also consistent with the meaning of “one or more,” “at least one” and “one or more than one.”

Unless indicated to be further limited, the term “plurality” as used herein means more than one, for example, two or more, three or more, four or more, and the like.

Where used herein, the term “about” refers to an approximately +/−10% variation from a given value.

As used herein, the recitation of numerical ranges by endpoints includes all numbers subsumed within that range including all whole numbers, all integers and all fractional intermediates (e.g., 1 to 5 includes 1, 1.5, 2, 2.75, 3, 3.80, 4, and 5 etc.).

Unless specified otherwise, the word “causes”, “causing”, “caused” and similar terms includes “directly causes” as well as “indirectly causes” through one or more than one intermediary molecule, step or mechanism.

Unless otherwise specified, “certain embodiments”, “various embodiments”, “an embodiment” and similar terms includes the particular feature(s) described for that embodiment either alone or in combination with any other embodiment or embodiments described herein, whether or not the other embodiments are directly or indirectly referenced and regardless of whether the feature or embodiment is described in the context of a method, product, use, composition, etcetera. None of Sections I, II, III and IV should be viewed as independent of the other Sections, but instead should be interpreted as a whole. Unless otherwise indicated, embodiments described in individual sections may further include any combination of features described in the other sections. Unless clearly not intended, definitions presented for terms in any section(s) are expressly incorporated into other section(s) as an alternative definition.

As used herein, a “polypeptide” (e.g. as used in the expression “transporter polypeptide”) is a chain of two or more amino acid residues (e.g. 2, 10, 50, 100, 200 or any other number of residues) linked by peptide bonds, including a peptide or a protein chain. As used herein, a “protein” comprises one or more polypeptides and may or may not further comprise non-polypeptide elements, including covalently or non-covalently attached co-factors, metals, organic compounds, lipids, carbohydrates, nucleic acids and/or other biomolecules or molecular entities. As such, the term “protein” expressly encompasses, without limitation, the term “peptide”. As such, a “region”, “portion” or “domain” of a protein may consist or comprise of such non-polypeptide elements. A protein may comprise 1, 2, 3, 4, 5, 6, 7, 8, 9, 10 or more than 10 polypeptide chains in covalent and/or non-covalent association. Non-limiting examples of non-covalent interactions include hydrogen bonds, hydrophobic interactions and/or electrostatic interactions. A non-limiting example of a covalent bond between polypeptides is a disulfide bridge.

As used herein, “nucleic acid”, “polynucleotide”, “oligonucleotide”, “nucleic acid sequence”, “nucleotide sequence”, and similar terms refer to polymers of bases typically linked by a sugar-phosphate backbone, and includes DNA or RNA of genomic or synthetic origin which can be single- or double-stranded, and represent a sense and/or antisense strand. Unless otherwise indicated, a particular nucleic acid sequence of this invention encompasses complementary sequences, in addition to the sequence explicitly indicated. Unless otherwise specified, a “nucleic acid”, “oligonucleotide”, “polynucleotide”, “DNA”, “RNA” and similar terms, can be double stranded or single stranded.

“Conservative variant”, “conservatively modified variants” and similar phrases apply to both amino acid and nucleic acid sequences. With respect to particular nucleic acid sequences, conservatively modified variants refers to those nucleic acids which encode identical or essentially identical amino acid sequences, or where the nucleic acid does not encode an amino acid sequence, to essentially identical sequences. Because of the degeneracy of the genetic code, a large number of functionally identical nucleic acids encode any given protein. For instance, the codons GCA, GCC, GCG and GCU all encode the amino acid alanine. Thus, at every position where an alanine is specified by a codon, the codon can be altered to any of the corresponding codons described without altering the encoded polypeptide. Such nucleic acid variations are “silent variations”, which are one species of conservatively modified variations. Every nucleic acid sequence herein which encodes a polypeptide also describes every possible silent variation of the nucleic acid. One of skill will recognize that each codon in a nucleic acid (except AUG, which is ordinarily the only codon for methionine, and TGG, which is ordinarily the only codon for tryptophan) can be modified to yield a functionally identical molecule. Accordingly, each silent variation of a nucleic acid that encodes a polypeptide is implicit in each described sequence.

As for amino acid sequences, individual substitutions, deletions and/or additions to a nucleic acid, peptide, polypeptide, or protein sequence which alter, add or delete a single amino acid or a small percentage of amino acids in the encoded sequence is a “conservatively modified variant” where the alteration results in the substitution of an amino acid with a chemically similar amino acid. Conservative substitution tables providing functionally similar amino acids are well known in the art. Such conservatively modified variants are in addition to and do not exclude polymorphic variants, interspecies homologues and alleles. Without limitation, the following eight groups each contain amino acids that are conservative substitutions for one another:
1) Alanine (A), Glycine (G);
2) Aspartic acid (D), Glutamic acid (E);
3) Asparagine (N), Glutamine (Q);
4) Arginine (R), Lysine (K);
5) Isoleucine (I), Leucine (L), Methionine (M), Valine (V);
6) Phenylalanine (F), Tyrosine (Y), Tryptophan (W);
7) Serine (S), Threonine (T); and
8) Cysteine (C), Methionine (M).

An amino acid sequence which comprises at least 50, 60, 70, 75, 80, 81, 82, 83, 84, 85, 86, 87, 88, 89, 90, 91, 92, 93, 94, 95, 96, 97, 98, 99 or 100% amino acid sequence identity to a specified reference sequence (e.g. a full-length reference sequence) is also a “conservatively modified variant” so long as it retains a specified activity or fraction of said activity. Sequence identity can be determined using the methods described herein, for example, aligning two sequences using BLAST, ALIGN, or another alignment software or algorithm known in the art using default parameters.

Unless otherwise specified, the term “subject” (or “patient” or “individual”, if used) refers to an animal. In some embodiments, the subject is a vertebrate. In some embodiments, the subject is a mammal. Without limitation, the mammal may be a laboratory mammal (e.g., mouse, rat, rabbit, hamster, non-human primate, mammal disease model, and the like) or may be an agricultural mammal (e.g., equine, ovine, bovine, porcine, camelid, and the like) or a domestic mammal (e.g., canine, feline, and the like). In some embodiments, the subject is a human.

### II. Gene Delivery System

Disclosed herein is a system for delivering a payload gene. The system comprises a Bifidobacterium sp. bacterium comprising a plasmid and a transporter nucleic acid. The transporter nucleic acid is in operative association with a first promoter and a first terminator configured to express the transporter nucleic acid in the bacterium. The transporter nucleic acid encodes a transporter polypeptide comprising, in an amino-terminal to carboxy-terminal order, a bacterial secretion signal peptide, a DNA-binding domain, and a cell penetrating peptide. The DNA-binding domain is configured for association with the plasmid to form a polypeptide-plasmid complex. The bacterial secretion signal peptide is configured for secretion of the polypeptide-plasmid complex from the bacterium. The cell penetrating peptide is configured for importing the polypeptide-plasmid complex into a eukaryotic cell (e.g. a mammalian cell, human cell, or the like). The plasmid comprises a payload nucleic acid having a sequence encoding a payload (e.g. a payload protein or RNA), the payload nucleic acid in operative association with a second promoter and a second terminator configured to express the payload nucleic acid in the eukaryotic cell and to produce the payload.

Non-limiting examples of Bifidobacterium spp. include B. adolescentis, B. angulatum, B. animalis, B. asteroides, B. bifidum, B. boum, B. breve, B. catenulatum, B. choerinum, B. coryneforme, B. cuniculi, B. denticolens, B. dentium, B. gallicum, B. gallinarum, B. indicum, B. inopinatum, B. infantis, B. longum, B. magnum, B. merycicum, B. minimum, B. pseudocatenulatum, B. pseudolongum, B. pullorum, B. ruminantium, B. saeculare, B. subtile, B. thermacidophilum, B. thermophilum and B. tsurumiense, and alternative embodiments include one or more of the foregoing. In some embodiments, the bacterium is Bifidobacterium longum. In some embodiments, the bacterium is Bifidobacterium longum subsp. longum.

As used herein, “plasmid” means a DNA molecule that is physically separated from chromosomal DNA and which can replicate independently. For example, a plasmid may be a circular double-stranded DNA molecule. The plasmid may be an “expression vector”, which refers to a recombinant vector (such as a plasmid) comprising operatively linked polynucleotide sequences that facilitate expression of a coding sequence in a particular host organism (e.g., a bacterial or eukaryotic expression vector). Expression vectors may comprise an “expression cassette” comprising a promoter operatively linked to a coding sequence followed by a transcription termination site (i.e., terminator). An expression cassette may also comprise one or more cloning sites or multiple cloning sites at desired locations within the cassette to permit the introduction or removal of sequences, into and out of, the cassette, respectively. Plasmids may comprise any number of expression cassettes, for example, at least or only one, two, three or more expression cassettes. Expression vectors may comprise at least, or consist of only, one, two, three or more prokaryotic expression cassettes for expression in Gram-positive bacteria, Gram-negative bacteria or combinations thereof. Expression vectors may alternatively or further comprise at least, or consist of only, one, two, three or more eukaryotic (e.g. mammalian, human, and the like) expression cassettes for expression in eukaryotic (e.g. mammalian, human, and the like) cells. A plasmid may have one or multiple origins of replication, including for example an origin of replication suitable for replication in the target or host cell in which expression of the coding sequence is intended. For example, the plasmid may have one or multiple origins of replication for replication in a bacterial cell (e.g. a Bifidobacterium sp. bacteria) and/or may have one or multiple origins of replication for replication in a eukaryotic (e.g. mammalian) cell (e.g. a human cell). Plasmids may also include a ribosomal binding site and/or other sequences. In certain embodiments, the transporter nucleic acid forms part of the plasmid. In certain embodiments, the transporter nucleic acid is separate from the plasmid (e.g. in the bacterial chromosome or in a second plasmid). In some embodiments, the plasmid is up to 16 kb in size.

Plasmids suitable for bacterial and/or eukaryotic (e.g. mammalian) applications are well known in the art, and are routinely designed and developed for particular purposes. A number of examples are described in WO/2015/120541 and WO/2015/120542. A non-limiting example of a plasmid that is suitable for expression of polypeptides in bacteria and vertebrates (e.g. mammals) is pFRG3.5-CMV-GLuc (SEQ ID NO: 43; Table 1), which is designed to express a non-limiting example of a transporter polypeptide “HTP” (arabinosidase signal sequence, Hu DNA-binding domain, Trans-Activator of Transcription (Tat) transduction domain; SEQ ID NO: 2) in the bacterium, to be bound by the Hu DNA-binding domain of HTP once expressed and to then be transported to the eukaryotic cell (e.g. mammalian cell) for expression and production of a payload, namely secreted Gaussia luciferase).

Many other plasmids are known that may be used/adapted, a number of non-limiting examples of which are described in WO/2015/120541 and WO/2015/120542, including (without limitation): pMW211, pBAD-DEST49, pDONRP4-P1R, pENTR-PBAD, pENTR-DUAL, pENTR-term, pBR322, pDESTR4-R3, pBGS18-N9uc8, pBS24Ub, pUbNuc, pIXY154, pBR322DEST, pBR322DEST-PBAD-DUAL-term, pJIM2093, pTG2247, pMEC10, pMEC46, pMEC127, pTX, pSK360, pACYC184, pBOE93, pBR327, pDW205, pKCL11, pKK2247, pMR60, pOU82, pR2172, pSK330, pSK342, pSK355, pUHE21-2, pEHLYA2-SD. See, for example, Stritzker, et al. Intl. J. Med. Microbiol. Vol. 297, pp. 151-162 (2007); Grangette et al., Infect. Immun. vol. 72, pp. 2731-2737 (2004), Knudsen and Karlstrom, App. and Env. Microbiol. pp. 85-92, vol. 57, no. 1 (1991), Rao et al., PNAS pp. 11193-11998, vol. 102, no. 34 (2005). Those skilled in the art, in light of the teachings of this disclosure, will understand that alternative plasmids may be used, or that the above plasmids may be modified in order to combine sequences as desired. For example, plasmids may be modified by inserting additional origins of replication, or replacing origins of replication, introducing expression cassettes comprising suitable promoter and termination sequences, adding one or more than one DNA binding sequence, DNA recognition site, or adding sequence(s) encoding payload polypeptides/proteins/RNAs as described herein, other products of interest, polypeptides of interest or proteins of interest, or a combination thereof. In some embodiments adjacent functional components of a plasmid may be joined by linking sequences.

Without limitation, a “coding sequence” as used herein includes a nucleotide sequence that codes for a polypeptide and (in such a case) is at least bounded by a start codon and a stop codon. A coding sequence also includes nucleic acid sequences which encode RNA payloads. As used herein, a nucleic acid sequence which “encodes” (or “codes” for) a payload (which may also be referred to herein as a “product of interest” or “cargo”) means that said nucleic acid sequence comprises a coding sequence for said payload. When used in the context of “encoding” a product or domain which is manufactured in vivo through an precursor or intermediate (e.g. where the product or domain can be manufactured via a post-translational modification), a nucleic acid which “encodes” said product or domain includes nucleic acids that comprise the nucleotide sequence of said precursor or intermediate. This is because the information for the post-translationally modified product or domain is contained within the sequence of the precursor/intermediate. Non-limiting examples of post-translational modifications include signal peptide processing, pro-peptide processing, protein folding, disulfide bond formation, glycosylation, carbonylation, gamma carboxylation, and beta-hydroxylation, oxidation, myristoylation, palmitoylation, isoprenylation, prenylation, glypiation, lipoylation, flavin attachment, heme C attachment, phosphopantetheinylation, retinylidene Schiff base formation, diphthamide formation, ethanolamine phosphoglycerol attachment, hypusine formation, acylation, acetylation, formylation, alkylation, methylation, amidation, amino acid addition, arginylation, polyglutamylation, poyglycylation, butryrylation, malonylation, hydroxylation, iodination, nucleotide addition, phosphate ester (O-linked) or phosphoramidate (N-linked) formation, phosphorylation, adenylylation, propionylation, pyroglutamate formation, S-glutathionylation, S-nitrosylation, S-sulfenylation, succinylation, sulfation, glycation, carbamylation, carbonylation, proteolytic cleavage, racemization and protein splicing. If used herein, the term “gene” (e.g. in “payload gene”) refers to a “coding sequence” such that a gene may encode a peptide, polypeptide, protein or RNA, and may include polycistronic coding sequences (e.g. separated by IRES) or a coding sequence that comprises one or more self-cleaving sequence(s) (e.g. 2A self cleaving peptide, such as P2A and the like).

As noted above, the transporter nucleic acid (or transporter nucleic acid sequence) is in operative association with a first promoter and a first terminator configured to express the transporter nucleic acid (or transporter nucleic acid sequence), and thus produce the transporter polypeptide, in the bacterium.

As used herein, the term “express” or “expression” in the context of expressing a nucleic acid or a polypeptide refers to transcription of the nucleic acid. When the nucleic acid encodes a polypeptide (or polypeptides), then “expression” also refers to translation (as well as any post-translational processing) in manufacture of the polypeptide/protein product encoded by the nucleic acid.

A “promoter” is a DNA region, typically but not exclusively 5′ of the site of transcription initiation, sufficient to confer accurate transcription initiation. The promoter typically contains regions of DNA that are involved in recognition and binding of RNA polymerase and other proteins or factors to initiate transcription. In some embodiments, a promoter is constitutively active, while in alternative embodiments the promoter is conditionally active (e.g., where transcription is initiated only under certain physiological conditions). Conditionally active promoters may thus be “inducible” in the sense that expression of the coding sequence can be controlled by altering the physiological condition. Non-limiting examples, of potential inducible promoters include, but are not limited to, IPTG inducible promoters, e.g. lacUV5 promoter (Moffatt, B. A., and Studier, F. W. (1986) J. Mol. Biol. 189, 113-130), teracycline inducible promoters (Gatz, C., 1997, Ann. Rev. Plant Physiol. Plant Mol. Biol. 48, 89 108), steroid inducible promoters (Aoyama, T. and Chua, N. H., 1997, Plant J. 2, 397-404) and ethanol inducible promoters (Salter, M. G., et al., 1998, Plant Journal 16, 127-132; Caddick, M. X., et al., 1998, Nature Biotech. 16, 177 180). Any promoter described herein (e.g. the first promoter or the second promoter, etc., as defined below) may be natural or may be artificial. For example, but without limitation, an artificial promoter may include multiple promoters.

A “terminator” or “transcription termination site” refers to a 3′ flanking region of a gene or coding sequence (e.g. a viral genome or a payload gene, genes or sequence) that contains nucleotide sequence(s) which regulate transcription termination and typically confer RNA stability.

As used herein, “operatively linked”, “in operative association” and similar phrases, when used in reference to nucleic acids, refer to the linkage of nucleic acid sequences placed in functional relationships with each other. For example, an operatively linked promoter sequence, open reading frame and terminator sequence results in the accurate production of an RNA molecule. In some aspects, operatively linked nucleic acid elements result in the transcription of an open reading frame and ultimately the production of a polypeptide (i.e., expression of the open reading frame).

The first promoter (which may be referred to elsewhere as the “bacterial promoter”) may be any promoter (or plurality of promoters) that initiates transcription of the transporter nucleic acid sequence in the bacterium. Promoters operative in various bacteria are well-known. Non-limiting examples of constitutive and inducible promoters that may be used for expression of a product of interest within some bacteria, including Bifidobacterium spp., may be found in Sun et al. (2012, Applied Environ Micrbiol 78:5035-5042) and include constitutive promoters, for example, Pₕᵤₚ (a promoter from a gene encoding a histone-like protein; also referred to as “Hup” promoter), P_{gap} (a promoter from a gene encoding glyceraldehydes 3 phosphate dehydrogenase), P_{amy} (a promoter from the gene encoding alpha-amylase), the promoter from the gene encoding 16S rRNA, Pₕₑₗₚ, the lambda phage promoter P_{R}P_{L}, and inducible promoters from the gene encoding alpha-galactosidase (induced in the presence of raffinose), heat, ethanol, osmotic induced promoters, and the arabinose inducible araC-P_{BAD} expression system. In certain embodiments, the first promoter is 16S rRNA promoter (or “RB Promoter”, an endogenous constitutive Bifidobacterium-specific ribosomal promoter, e.g. see nucleotides 3363-3436 of SEQ ID NO: 43) or a Hup promoter (e.g. a Bifidobacterium-specific hup gene promoter).

The first terminator may be any terminator that is functional with the bacterial promoter in the bacterium, and in some embodiments the first terminator may be bacteria-specific. Non-limiting examples of bacterial terminators include HU terminator, hup gene terminator and 16S rRNA terminator. In some embodiments, the first terminator is SynS terminator (endogenous Bifidobacterium-specific ribosomal terminator; e.g. see nucleotides 3839-3880 of SEQ ID NO: 43) or hup gene terminator.

In some embodiments, the first promoter and the first terminator are the RB promoter and the SynS terminator, respectively. In some embodiments, the first promoter and the first terminator are the hup gene promoter and terminator, respectively.

In some embodiments, the plasmid may further comprise an antibiotic or chemical resistance gene (e.g. spectinomycin resistance gene) in operative association with a further promoter and terminator configured to express the resistance gene in the bacteria. Such a further promoter and terminator may be the same or different from the first promoter and terminator, and may be any as defined above for the first promoter and the first terminator, respectively.

As noted above, the plasmid comprises a payload nucleic acid in operative association with a second promoter and a second terminator configured to express the payload nucleic acid in the eukaryotic cell.

The second promoter may be any promoter that initiates transcription of the payload nucleic acid (or payload nucleic acid coding sequence) in the eukaryotic cell (e.g. in the mammalian cell). In some embodiments, the second promoter is a eukaryotic promoter. In some embodiments, the second promoter is a viral promoter. In some embodiments, the second promoter is a bacteriophage-derived promoter. Constitutive and/or inducible promoters that may be used for expression of a product of interest within eukaryotic cells, including but not limited to tumour cells or other cells in the tumour microenvironment, are known and too numerous to list (e.g. a current list is provided in The Eukaryotic Promoter Database, Dreos et al., 2015, Nucl. Acids Res. 43 (D1): D92-D96, available online). Non-limiting examples of promoters for the second promoter include CMV (cytomegalovirus) promoter (e.g. nucleotides 2166-2385 of SEQ ID NO: 43), SV40 (simian virus 40) promoter, UBC (human ubiquitin C) promoter, EF1A (human elongation factor 1alpha) promoter, PGK (mouse phosphoglycerate kinase 1) promoter, CAG (CMV enhancer fused to the chicken beta-actin) promoter, CHEF-1alpha (Chinese hamster elongation factor 1alpha) promoter, or a tetracycline- or IPTG-inducible promoter. The second promoter may by a promoter targeted by RNAP III, e.g. for expression of small RNA payloads for RNA interference (such as shRNAs); non-limiting examples of such promoters are U6 promoter (endogenous snRNA promoter), H1 promoter (endogenous ncRNA), and the like. The second terminator may be any terminator that functions with the second promoter. For example, but without limitation, the second terminator may be TK polyA (e.g. see nucleotides 3036-3356 of SEQ ID NO: 43). In some embodiments, the second promoter comprises the CMV promoter or the SV40 promoter and the second terminator is TK polyA. In some embodiments, but without limitation, the second promoter comprises CMV promoter, SV40 promoter, UBC promoter, EF1A promoter, PGK promoter, CAG promoter, CHEF-1alpha promoter, a tetracycline- or IPTG-inducible promoter, U6 promoter, or H1 promoter.

In certain embodiments, the second promoter is non-specific. In other embodiments, the second promoter is tissue-specific or cell-specific. Enhancers may be used to enhance the activity of promoters. For example the activity of the TYR promoter has been enhanced by also including the human tyrosinase distal element (TDE) as well as by including a mouse enhancer elements (e.g. the TETP promoter construct or the Tyrex2 promoter) (Pleshkan et al., 2011, ibid.). In alternative embodiments, the second promoter is any one or more of the exemplary promoters listed above for this element.

The plasmid may further comprise a Kozak sequence and/or an IRES sequence.

In some embodiments, e.g. where the second promoter comprises the CMV promoter, the SV40 promoter, or any other second promoter listed above or elsewhere described herein, the second promoter and the coding sequence of the payload nucleic acid (i.e. the payload coding sequence) are positioned on the plasmid with a Kozak sequence between them. In some embodiments, the second promoter and the payload coding sequence are positioned on the plasmid without a Kozak sequence between them. In some embodiments, the payload nucleic acid comprises an internal ribosome entry site (IRES) sequence or encodes a 2A self cleaving peptide sequence(s) or other sequence that induces ribosome skipping during translation. In some embodiments in which the payload nucleic acid comprises an IRES sequence, a Kozak sequence is absent. The Kozak sequence is recognized by the mammalian ribosome as the translational start site so removing the Kozak sequence would favour translation mediated by the IRES located in the 5′UTR (“untranslated region”). In some embodiments in which a Kozak sequence is positioned as defined above, the payload nucleic acid comprises an internal ribosome entry site (IRES) sequence. A non-limiting example of an IRES sequence is nucleotides 6300-6878 of SEQ ID NO:45. 2A self cleaving peptide sequences are known (e.g. T2A, P2A, E2A, F2A, and the like).

In some embodiments, the second promoter is a ribosomal RNA gene promoter recognized by an RNA polymerase that is native to the eukaryotic cell or target tissue/cell. In these embodiments, the second terminator may comprise at least one “Sal box” sequence motif (e.g. 1, 2, 3, 4, 5, 6, 7, 8, 9, 10 or more that 10 Sal box sequence motifs). The Sal box motif if found in the rRNA gene (e.g. in humans, mice etc.) and is recognized by a DNA-binding protein called Transcription Termination Factor 1 (TTF-1). TTF-1 binding to the Sal box sequence motif is sufficient for termination of RNA polymerase I transcription and release of the nascent RNA chains from the elongation machinery. Similar sequence elements which are functionally analogous to the murine motif are present in the human rRNA gene. Multiple copies of the Sal box motif naturally exist within the rRNA gene terminator region, adjacent to pyrimidine-rich sequences which play a role processing the nascent transcript into authentic pre-rRNA termini. Single Sal box sequence motifs when bound by TTF-1 have been shown to terminate transcription in both cell-free transcription assays and in transfection experiments, which shows that only a single Sal box would be needed as a terminator signal.

In some embodiments, the second promoter is a bacteriophage-derived promoter. Non-limiting examples of bacteriophage-derived promoters include T7 promoter, T3 promoter and SP6 promoter. In these embodiments, the second terminator may be a rho-independent terminator (also known as an “intrinsic promoter”). The bacteriophage-derived promoters require the presence of a compatible RNA polymerase (i.e. an RNA polymerase that recognizes the bacteriophage-derived promoter), which is not natively expressed in mammalian cells. For example, T7, T3 and SP6 require T7 RNA polymerase, T3 RNA polymerase and SP6 RNA polymerase, respectively. Accordingly, in embodiments that use a bacteriophage-derived promoter, the plasmid further comprises an RNA polymerase nucleic acid in operative association with a third promoter and a third terminator configured to express a heterologous or exogenous RNA polymerase, specific for the bacteriophage-derived promoter, in the mammalian cell (e.g. the tumour cell). The phrase “RNA polymerase that recognizes the bacteriophage-derived promoter” includes without limitation the cognate RNA polymerase that is functional with the particular bacteriophage-derived promoter. Other bacteriophage-derived promoters and their cognate RNA polymerase are known. The third promoter and the third terminator may be any combination of promoter and terminator, natural or artificial, that is functional in the eukaryotic cell (e.g. in the mammalian cell) to express the RNA polymerase, e.g. a constitutive or inducible promoter. In some embodiments, but without limitation, the third promoter comprises CMV promoter, SV40 promoter, UBC promoter, EF1A promoter, PGK promoter, CAG promoter, CHEF-1alpha promoter, or a tetracycline- or IPTG-inducible promoter. In some embodiments, the third promoter is non-specific. In some embodiments, the third promoter is tissue-specific. In some embodiments, the third terminator comprises TK polyA.

In some embodiments, the payload nucleic acid comprises a single payload coding sequence. In some embodiments, the payload nucleic acid comprises multiple payload coding sequences. The latter embodiments may include additional promoters and terminators configured to express the additional payloads in the eukaryotic cell (e.g. in the mammalian cell). The additional promoters and terminators may be any combination of promoter and terminator, natural or artificial, that is functional in the eukaryotic cell (e.g. in the mammalian cell) to express the additional payload in the eukaryotic cell, e.g. a constitutive or inducible promoter. In some embodiments, but without limitation, the additional promoter comprises CMV promoter, SV40 promoter, UBC promoter, EF1A promoter, PGK promoter, CAG promoter, CHEF-1alpha promoter, or a tetracycline- or IPTG-inducible promoter. In some embodiments, the additional promoter is non-specific. In some embodiments, the additional promoter is tissue-specific. In some embodiments, the additional terminator comprises TK polyA.

In some embodiments, the payload nucleic acid comprises a plurality of payload coding sequences, wherein the payload nucleic acid is operatively associated with a single promoter and terminator for expression in the eukaryotic cell (e.g. the mammalian cell), and wherein each payload coding sequence is separated by an IRES element. In some embodiments, the payload nucleic acid comprises a plurality of payload coding sequences, wherein the payload nucleic acid is operatively associated with a single promoter and terminator for expression in the eukaryotic cell (e.g. the mammalian cell), and wherein each payload coding sequence is separated by a sequence that causes ribosome skipping during translation (e.g. sequence(s) that encode 2A self cleaving peptide(s)). In some embodiments, the payload nucleic acid comprises a plurality of payload coding sequences and each payload coding sequence is operatively associated with a separate promoter and terminator for expression in the eukaryotic cell (e.g. the mammalian cell). In some embodiments, the payload nucleic acid comprises a combination of multiple promoters, terminators and IRES sequences or sequences that induce ribosome skipping (i.e. a combination of one or more of the foregoing). In some embodiments, a portion of the plurality of payload-encoding sequences may be separated on separate plasmids as defined herein; the separate plasmids may each be in a separate bacterium as defined herein.

As noted above, the transporter nucleic acid encodes a transporter polypeptide (also referred to as a “hybrid transport protein” or “novel hybrid protein”) comprising, in an amino-terminal to carboxy-terminal order, a bacterial secretion signal peptide (BSSP), a DNA-binding domain (DBD), and a cell penetrating peptide (CPP). The DNA-binding domain is configured for association with the plasmid to form a polypeptide-plasmid complex. The bacterial secretion signal peptide is configured for secretion of the polypeptide-plasmid complex from the bacterium. The cell penetrating peptide is configured for importing the polypeptide-plasmid complex into a eukaryotic cell (e.g. a mammalian cell).

The polypeptide-plasmid complex associates through direct or indirect binding (covalent or non-covalent) of the DNA binding domain to the plasmid. While a wide range of suitable domains and their complementary DNA binding sequences will be readily identified by those skilled in the art, a number of non-limiting illustrative examples are disclosed in U.S. Pat. No. 6,007,988. In certain embodiments hereof, the DNA binding domain is (or is derived from) MerR, Zinc finger, or Histone-like DNA binding protein or is (or is derived from) the Hu protein or is (or is derived from) a homeobox DNA binding protein. It will be understood that the Hu protein is generally considered a homeobox-like protein. Additional DNA binding domains and variants (e.g. conservative variants) will be readily identified by those skilled in the art using available databases, screening methodologies and well known techniques. Suitable DNA binding domains may be of any general type, including but not limited to helix-turn-helix, Zinc finger, leucine zipper, winged helix, winged helix turn helix, helix loop helix, HMG box, Wor 3 and RNA guided binding domains. Some non-limiting illustrative examples of DNA binding proteins whose DNA binding domains may be utilized in embodiments include histones, histone-like proteins, transcription promoters, transcription repressors and transcriptional regulators, which may be drawn from a wide range of alternate sources and operons.

The DNA binding domain in the transporter polypeptide may bind a nucleic acid sequence of the plasmid in a non-specific manner, or the DNA binding domain may be specific for a corresponding DNA recognition site or consensus sequence in the plasmid. Specific DNA binding domain-nucleic acid recognition site combinations are known in the art. Non-limiting examples of a DNA binding domain that binds with a specific nucleic acid sequence includes: a MerR DNA binding domain (e.g., but not limited to, SEQ ID NOs: 4 and 5 for nucleotide and amino acid sequences, respectively), in which case the plasmid further comprises a MerR DNA recognition site (e.g., but not limited to SEQ ID NO: 10); and a zinc finger DNA binding domain (e.g., but not limited to, SEQ ID NOs: 8 and 9 for nucleotide and amino acid sequences, respectively), in which case the plasmid further comprises a zinc finger DNA recognition site (e.g., but not limited to, SEQ ID NO: 11). Another non-limiting sequence-specific DNA binding domain is the Lac repressor DBD, which is well known to bind its cognate recognition sequence when expressed in bacteria in commercial PET vectors. A non-limiting example of anon-specific DNA binding domain includes a Hu DNA binding domain (e.g., but not limited to, SEQ ID NO: 6 and 7 for nucleotide and amino acid sequences, respectively). In embodiments in which the transporter nucleic acid is separate from the plasmid (e.g. in the bacterial chromosome), the DNA binding domain may be specific for a corresponding DNA recognition site on the plasmid.

In alternative embodiments, the DNA binding domain comprises one or more of the above-referenced DNA binding domains. The transporter polypeptide may comprise one or a plurality of DNA binding domains and the plasmid may comprise one or a plurality of DNA recognition sites. For example, the transporter polypeptide may comprise 1, 2, 3, 4, 5, 6, 7, 8, 9,10, 12, 14, 16, 17, 18, 19, 20 or more DNA binding domains, or any number therebetween. Similarly, each plasmid may comprise 1, 2, 3, 4 or more than 4 DNA recognition sites.

As used herein, “bacterial secretion signal peptide” or “BSSP” means a sequence/peptide that functions in the export of an attached polypeptide out of a bacterial cell and into the extracellular environment of the bacteria, regardless whether or not the sequence was obtained from a bacteria, is artificial or from anon-bacterial organism. Bacterial secretion signal peptides typically have a tripartite structure with an N-terminal region encompassing one to three positively charged amino acid residues (N domain), a hydrophobic core region consisting of 10-15 residues (H domain), and a more polar C-terminus, which, for specific secretion pathways like the Sec-dependent pathway, can contain the signal peptidase cleavage site (C domain) (Driessen & Nouwen, 2008, Annu Rev Biochem 77:643-647). Even though these domains show little sequence conservation, the presence of a bacterial secretion signal peptide can be readily determined by one of skill in the art using appropriate analytical tools that aid in mapping out these domains and in determining an appropriate secretion signal peptide. For example, signal sequence prediction software such as SignalP 4.1 (Petersen et al., 2011, Nature Methods 8, 785-786) may be used to map out and determine a bacterial secretion signal sequence. Additionally, a bacterial secretion signal peptide may be determined by selecting a secretion signal peptide based on sequence identity relative to a known bacterial secretion signal peptide, provided that the bacterial secretion signal peptide retains the function of exporting a sequence of interest that is fused to the bacterial secretion signal peptide (e.g. a transporter polypeptide).

The bacterial secretion signal peptide may be any secretion signal peptide known or otherwise recognized by one of skill in the art. For example, Sun et al. (2012, Applied Environ Micrbiol 78:5035-5042) provide examples of bacterial secretion signal peptides, which may be used with Bifidobacterium spp. In certain embodiments, the bacterial secretion signal peptide comprises the signal peptide of the alpha-amylase of B. adolescentis INT-57, the signal peptide of beta-galactosidase, the signal peptide from B. breve Sec2, or the signal peptide from B. longum XynF. In certain embodiments, the bacterial secretion signal peptide comprises an alpha-L-arabinosidase signal peptide (e.g. from Bifidobacterium longum). Additional bacterial secretion signal peptides that may be used are those associated with Sec-dependent Protein Translocation, ABC transporters or oligopeptide permease.

Characteristic bacterial secretion signal peptides of Sec-dependent protein translocation (Driessen & Nouwen, 2008, Ann Rev Biochem 77:643-667) have a tripartite structure (N Domain—N-terminal region with 1-3 positively charged amino acid residues; H Domain—hydrophobic core region with 10-15 residues; C Domain—more polar C-terminus usually encompassing the signal peptidase cleavage site). While these sequences may show little conservation, they can be conveniently predicted based on these properties. One group of bacterial secretion signal peptides harbour a YSIRK-G/S motif which may function in concert with a C-terminal cell wall sorting signal to increase efficiency of secretion and association with the cell envelope.

Bacterial secretion signal peptides may be associated with ABC transporters (ATP-Binding Cassette transporters) which are integral membrane proteins that actively transport molecules across the cell membranes using the energy derived from the hydrolysis of ATP to ADP (Fath and Kolter, 1993, Microbiol Rev 57(4): 995-1017). Examples of ABC transporters are described in Moussatova et al. (2008, Biochemica et Biophsyica Acta 1778: 1757-1771). Oligopeptide permeases (Opp) are a subfamily of ABC transporters that have been identified in a number of Gram-positive and Gram-negative bacteria.

Other bacterial secretion signal peptides may be used from proteins involved in the Tat Pathway (twin arginine signal peptides), Pseudopilin Export signals, Holins, Retention signals (see for example Sibbald et al., 2006, Microbiol and Molec Bio Rev 70(3):755-788; Filloux, 2010, J Bacteriol 192(15):3847-3849; Economou et al., 2006, Molec Microbiol 62(2): 308-319).

In certain embodiments, the bacterial secretion signal peptide is selected from any one of SEQ ID NOs: 13, 15 and 17, or a variant (including but not limited to a conservative variant) sequence that is about 50%, 51%, 52%, 53%, 54%, 55%, 6%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 13, 15 and 17, wherein the secretion signal sequence retains at least a portion (e.g. at least 25%, at least 50% or at least 75%) of the function of exporting the polypeptide:plasmid complex out of the bacteria, or which retains at least a portion (e.g. at least 25%, at least 50% or at least 75%) of the function of transforming a plasmid when linked to a DNA-binding domain (i.e. retrograde secretion). Exemplary nucleotide sequences which encode SEQ ID NOs: 13, 15 and 17 are shown in SEQ ID NOs: 12, 14 and 16, respectively, although any nucleotide sequence which encodes the desired sequence may alternatively be used, and are obtainable from codon tables. In certain embodiments, the bacterial secretion signal peptide comprises the amino acid sequence of SEQ ID NO: 13.

DNA sequences may or may not be codon-optimized for the particular bacteria. In some embodiments, one or more of the coding sequences in the plasmid (other than the payload coding sequences) are codon-optimized for expression in the bacterium. Without limitation, the transporter nucleic acid sequence may be codon-optimized for expression in a Bifidobacterium sp. (e.g. B. longum and the like).

As used herein, “importing” or “importation” into a eukaryotic cell (e.g. a mammalian cell or a human cell) means transporting a substance from the external environment of the eukaryotic cell across the cell membrane of the cell, and into the cell. A non-limiting example of importation includes transportation of a polypeptide-plasmid complex into a eukaryotic cell for expression of the payload nucleic acid coding sequence(s). In this disclosure, importation across the cell membrane of the eukaryotic cell is accomplished using a “cell penetrating peptide” (CPP). The term “CPP” is well understood as a class of peptides which are able to translocate across the cell membrane of a eukaryotic cell. Briefly, many CPPs are cationic and hydrophilic due to a plurality of arginine/lysine residues, while other CPPs are amphipathic or hydrophobic. The term CPP includes the transduction domain of TAT or “trans-activator of transcription” (e.g. HIV-1 TAT or any other TAT). For greater certainty, but without limitation, reference to the transduction domain of TAT, i.e. CPP(TAT), or any other CPP will be understood to mean not only the native sequence, but also a full range of sequence variants thereof which are suitable to carry out the desired function of the protein or protein domain in question. By way of example, a range of functional variants of the CPP(TAT) are described in Salomone et al. (2012, Journal of Controlled Release 163, 293-303). Other non-limiting examples of CPPs include the VP22 protein of Herpes Simplex Virus, and the protein transduction domain of the Antennapedia (Antp) protein as well as the protein transduction domains Rev, Pep1 and Transportan. In certain embodiments, the CPP domain is the domain described in Salomone et al., (2012, ibid). Table 1 includes a non-limiting selection of amino acid sequences for exemplary CPP domains, including SEQ ID NOs: 18-42. In some embodiments, the CPP amino acid sequence comprises one or more of SEQ ID NOs:18-42, or a variant (including but not limited to a conservative variant) with 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98% or 99% sequence identity thereof which still retains CPP activity (e.g. as determined by plasmid transfection efficiency of at least 25%, at least 50% or at least 75% of the CPP from which the variant is derived). In some embodiments, the CPP domain is a peptide of 6-30 amino acids in length (e.g. 6, 7, 8, 9, 10, 11, 12, 13, 14, 15, 16, 17, 18, 19, 20, 21, 22, 23, 24, 25, 26, 27, 28, 29 or 30), with at least 3, 4, 5, 6, 7, 8, 9, 10, 11 or 12 Arg residues and a net positive charge. In some embodiments, the CPP domain comprises a poly-arginine peptide of 4-12 residues. A person of skill in the art would have no difficulty in converting an amino acid sequence into a nucleotide sequence which encodes the amino acid sequence for expression in a particular organism, without requiring any undue experimentation or inventive skill. Accordingly, nucleotide sequences to encode amino acid sequences for any of SEQ ID NOs:18-42 are obtainable from codon tables given these amino acid sequences. For example, but without limitation, a DNA sequence encoding SEQ ID NO:18 is shown in nucleotides 3803-3838 of SEQ ID NO:43.

In some embodiments, the transporter polypeptide comprises the amino acid sequences of all three of SEQ ID NOs: 7, 13 and 18. In some embodiments, the amino acid sequence of the transporter polypeptide comprises SEQ ID NO: 2, or a variant (including but not limited to a conservative variant) with 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79% 80%, 81%, 82%, 83%, 84%, 85%, 86%, 87%, 88%, 89%, 90%, 91%, 92%, 93%, 94%, 95%, 96%, 97%, 98% or 99% sequence identity thereof which still retains at least 25%, 30%, 40%, 50%, 60%, 70% or 80% bacterial secretion activity (in vitro) and also retains at least 25%, 30%, 40%, 50%, 60%, 70% or 80% mammalian cell transfection efficiency (in vitro). Nucleotide sequences to encode the amino acid sequence of the transporter polypeptide, i.e. the transporter nucleic acid, are obtainable from codon tables given the amino acid sequence of the transporter polypeptide. For example, but without limitation, a DNA sequence encoding SEQ ID NO:2 is shown in nucleotides 3437-3838 of SEQ ID NO:43.

In some embodiments, the plasmid may be adapted to reduce unnecessary genetic elements. For example, the transporter nucleic acid sequence need not be carried by the plasmid that encodes the payload, but may be encoded on the bacterial chromosome. Accordingly, in some embodiments, the payload nucleic acid is contained on the plasmid and the chromosome of the bacteria contains the transporter nucleic acid. In other embodiments, the plasmid comprises the transporter nucleic acid sequence. In bacteria where transcription and translation occur simultaneously, the transporter polypeptide may preferentially bind to and secrete the same plasmid that transcribes it.

In a further example, vestigial sequences may be removed, such as unnecessary origins of replication, promoters, enhancers and/or termination signals not used in the bacteria or eukaryotic cell. For example, an E. coli origin of replication (useful for growth or manipulation of the plasmid in E. coli) is not used in Bifidobacterium spp. or the subject, and may be deleted. As a further example, a specific DNA binding domain recognition site (e.g. a zinc finger recognition site), if present, may be removed in embodiments where the DNA binding domain is non-specific (e.g. when using a Hu DBD).

In some embodiments, the bacterium may comprise multiple copies of the transporter nucleic acid sequence to provide increased transporter polypeptide expression for transport of larger plasmids. In some embodiments, the first promoter operably linked to the transporter nucleic acid is a strong promoter (e.g. a constitutively active promoter endogenous to the particular bacteria or otherwise as known in the art).

In some embodiments, the plasmid be optimized for expression. For example, an enhancer(s) may be selected for optimal expression in the eukaryotic cell or the payload(s) may be codon-optimized for expression in the particular eukaryotic cell (e.g. in a mammalian cell or a human cell).

As noted above, the plasmid comprises a payload nucleic acid for expression in the eukaryotic cell. The payload nucleic acid may encode a polypeptide payload (or multiple polypeptide payloads) and/or an RNA payload (or multiple RNA payloads) configured to have a desired effect (e.g. a diagnostic or therapeutic effect) in the eukaryotic cell, or to be useful in research.

In some embodiments, the payload nucleic acid encodes a soluble protein (e.g., but without limitation, a marker, such as GFP, an enzyme, immunomodulatory protein, a cytotoxin, an antigen from a pathogen, and the like). In some embodiments, the payload nucleic acid encodes a secreted protein. A non-limiting example of a plasmid designed to secrete a protein (Gaussia luciferase) is pFRG3.5-CMV-GLuc (SEQ ID NO:43; Table 1).

In some embodiments, the payload nucleic acid encodes a membrane protein. In some embodiments, the payload nucleic acid encodes an integral membrane protein. In some embodiments, the payload nucleic acid encodes a cell surface protein. In some embodiments, the payload nucleic acid encodes a membrane or membrane-associated protein comprising an extracellular domain.

Targeting proteins to the plasma membrane of eukaryotic cells (e.g. mammalian cells) for membrane attachment/association (e.g. for local delivery in colonic cells) or secretion (e.g. for systemic delivery) may be achieved in various ways. For example, preproteins containing an N-terminal endoplasmic reticulum (ER) signal peptide or a transmembrane segment(s) are inserted through the membrane of the ER, thereby directing the preprotein into the secretory pathway. During this process, the ER signal peptide interacts with the signal recognition particle (SRP), which in turn is recognized by the SRP receptor in association with the ER translocon. If the translating protein comprises transmembrane segment(s) (i.e a signal-anchor sequence or a stop transfer sequence/membrane-anchor sequence), then these segments will be embedded in the ER membrane to produce an integral membrane protein. After or simultaneous with insertion of the preprotein into the ER, the N-terminal ER signal peptide is cleaved from the preprotein by a signal peptidase. The ER membrane and any proteins segregated therein then migrate to the Golgi apparatus and then to secretory vesicles. Fusion of the secretory vesicles with the plasma membrane incorporates into the plasma membrane those membrane proteins embedded in the vesiclular membrane and also releases the contents of the vesicle into the extracellular environment (i.e. secretion). ER signal peptides and transmembrane segments are known and may be confirmed/predicted using available software (e.g. SignalP 4.1; MHMM, Krogh et al. Journal of Molecular Biology 2001; 305(3):567-580; OPCONS—Tsirigos et al. 2015 Nucleic Acids Research 43 (Webserver issue), W401-W407; TMpred—Hofmann & Stoffel 1993 Biol. Chem. Hoppe-Seyler 374,166; and the like). Certain membrane proteins (e.g. beta-barrels and the like) may use chaperones and other/additional mechanisms for translation and insertion into the plasma membrane. Alternative mechanisms for protein secretion also exist, e.g. post-translational secretion and unconventional protein secretion. Unless otherwise specified, the embodiments described herein are not limited to a particular construct or mechanism of secretion or membrane association.

In some embodiments, the membrane or membrane-associated protein is an integral membrane protein. In such embodiments, the payload nucleic acid sequence encodes a transmembrane segment(s) or transmembrane domain(s). In certain embodiments, the transmembrane segment acts as a signal-anchor sequence or stop transfer sequence/membrane-anchor sequence. The payload nucleic acid sequence may further encode an N-terminal ER signal peptide which is cleaved off as a result of insertion into the ER lumen. The orientation of an integral membrane protein in the plasma membrane is determined by the amino acid sequence encoded by the payload nucleic acid sequence, including the presence/absence of an N-terminal ER signal peptide, the net electrostatic charge flanking the transmembrane segments, and the length of the transmembrane segments. As a general rule, the flanking segment that carries the highest net positive charge remains on the cytosolic face of the plasma membrane and long hydrophobic segments (>20 residues) tend to adopt an orientation with a cytosolic C-terminus. The topology/orientation of membrane proteins can be predicted using available software (e.g.: MHMM; OPCONS; TMpred; and the like; each cited above).

The transmembrane domain may be a natural transmembrane domain from the membrane protein payload, a natural transmembrane domain from a heterologous membrane protein, or an artificial transmembrane domain. Without limitation, a natural or artificial transmembrane domain may comprise a hydrophobic α-helix of about 15 to about 23 amino acids (e.g. 15, 16, 17, 18, 19, 20, 21, 22, 23 or more than 23 residues), often with positive charges flanking the transmembrane segment. The transmembrane domain may have one transmembrane segment or more than one transmembrane segment.

In some embodiments, the membrane or membrane-associated protein is a peripheral membrane protein. Peripheral membrane proteins may associate with the outer leaflet of the plasma membrane by non-covalent association. For example, but without limitation, the peripheral membrane protein may comprise an amphipathic alpha-helix that associates with the membrane in a parallel orientation to the membrane plane through hydrophobic interactions (e.g. with the phospholipid tails of the membrane) and polar/electrostatic interactions (with the charged/polar phospholipid head groups). In another non-limiting example, the peripheral membrane protein may comprise a hydrophobic loop(s). In another non-limiting example, the peripheral membrane protein may interact with the plasma membrane through electrostic or ionic interactions (e.g. through a calcium ion and the like).

In some embodiments, the membrane or membrane associated protein is a lipid-anchored protein (also called a lipid-linked protein). Non-limiting examples of lipid-modification of proteins to produce lipid-anchored proteins include modification with fatty acids, isoprenoids, sterols, phospholipids, and glycosylphosphatidyl inositol (GPI) anchors. Without limitation, the lipid-anchored protein may comprise a GPI anchor (or any other lipid anchor), e.g. a GPI anchor or a non-GPI lipid anchor that is functional in the eukaryotic cell. Lipid modification sites are determined by the preprotein amino acid sequence encoded by the payload nucleic acid sequence. For example, in some embodiments, to produce a GPI-anchored protein, the payload nucleic acid sequence encodes an N-terminal ER signal peptide and/or a transmembrane segment(s) to target the protein to the ER and further encode a GPI signal peptide (e.g. to a C-terminal transmembrane segment). If present, the ER signal peptide is cleaved off as a result of insertion of the translating protein into the ER lumen. The GPI transamidase in the eukaryotic cell cleaves off the C-terminal transmembrane segment and transfers the protein to a preformed GPI-anchor. Various GPI signal peptides active in eukaryotic cells are known, e.g. folate receptor GPI signal peptide, and the like. More generally, in some embodiments, the payload nucleic acid sequence encodes an ER signal peptide and/or a transmembrane segment(s) which targets the protein to the ER and further encodes a lipid anchor signal (e.g. an amino acid sequence that when folded produces a site for post-translational modification in the eukaryotic cell by a fatty acid, isoprenoid, sterol, phospholipid or glycosylphosphatidyl inositol). Prospective GPI signal peptides may be confirmed as functional using GPI anchor prediction software (e.g. PredGPI; Pierleoni et al., BMC Bioinformatics 9:392, 2008) and confirmed as functional in the context of a chimeric protein using signal sequence prediction software (e.g. SignalP 4.1). Other lipid anchor signals are likewise known or determinable by software or routine testing. Where production of the lipid-anchored protein in the eukaryotic cell requires cleavage (e.g. removal of the C-terminal portion of GPI signal peptide), then the payload nucleic acid sequence encodes the pre-cleavage signal (e.g. a pre-cleavage GPI signal peptide).

In some embodiments, the payload nucleic acid encodes an extracellular domain and further encodes a transmembrane domain, lipid anchor or peripheral membrane protein domain for externally displaying the extracellular domain (e.g. fused to the transmembrane domain, lipid anchor or peripheral membrane protein domain) outside the cell membrane of the eukaryotic cell. In certain embodiments, the payload nucleic acid encodes an N-terminal ER signal peptide and an extracellular domain fused to a transmembrane domain. For example, but without limitation, the extracellular domain may comprise IL-12 or a functional fragment thereof, or comprises a fusion of IL-12 alpha and beta domains (e.g. human p35 and p40) or a fusion of functional fragments thereof. Human IL-12 is a soluble extracellular protein composed of alpha (p35) and beta (p40) domains, which associate together through covalent (disulfide bridge) and non-covalent interactions (Reitberger et al. (2017) J. Biol. Chem. 292(19):8073-8081). As a payload, IL-12 may be produced in the eukaryotic cell with one of the two domains secreted from the eukaryotic cell and the other domain linked to the membrane through linkage to a transmembrane domain, a lipid anchor or peripheral membrane protein (or domain thereof). Alternatively, a fusion of IL-12's two domains may be further fused to a transmembrane domain, a lipid anchor or a peripheral membrane protein (or domain thereof). In some embodiments, but without limitation, the extracellular domain comprises a fusion of IL-12 alpha and beta domains further fused to a transmembrane domain (e.g. as in SEQ ID NO:3). Each domain may be joined directly or through a peptide linker. Non-limiting peptide linkers include 1, 2, 3, 4, 5, 6, 7, 8, 9, 10, 11, 12, 13, 14, 15, 16, 17, 18, 19, 20 or more than 20 amino acid residues, wherein each residue in the peptide may independently be any amino acid. In some embodiments, the linker comprises predominantly Gly residues, with fewer Ser and/or Thr residues. The transmembrane domain may be any transmembrane domain. In some embodiments, the transmembrane domain is an insulin receptor transmembrane domain (e.g. human insulin receptor transmembrane domain; e.g. amino acids 547-577 of SEQ ID NO: 3). In some embodiments, the extracellular domain comprises amino acids 1-328 and 336-532 of SEQ ID NO:3. In some embodiments, the membrane or membrane-associated protein encoded by the payload nucleic acid comprises SEQ ID NO:3, or a variant (including but not limited to a conservative variant) with 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98% or 99% sequence identity thereof which still retains at least 25%, 30%, 40%, 50%, 60%, 70% or 80% IL-12 activity. Standard methods for evaluating IL-12 biofunctionality in vitro involve stimulation of cultured peripheral blood monocytes (PBMCs). For example, incubations of cultured PBMCs with purified IL-12 heterodimer or IL-12 fusion constructs, with cells expressing and secreting IL-12 heterodimer or fusion constructs or with cells expressing membranous IL-12 heterodimer or fusion constructs, allows interaction of IL-12 with cognate IL-12 receptors on relevant immune cell types (T cells, NK cells; subset of PBMC population) thereby leading to their stimulation/activation. IL-12-stimulated immune cells will express and secrete proinflammatory cytokines (eg. IFN-gamma, IL-2). IFN-gamma gene expression can be measured using qRT-PCR analysis of stimulated PMBC, for example. IFN-gamma secretion can be measured using ELISA-based analysis of stimulated PBMC growth media, for example. Alternatively, stimulated T cells can be assayed for the expression of cell surface activation markers using flow cytometry; such markers include CD137 (4-1BB), CD134 (OX40), and/or CD30. In some embodiments, the payload nucleic acid encodes SEQ ID NO:3.

The payload nucleic acid may also comprise, or alternatively comprise, other targeting signals. For example, epithelial cells can be polarized (or asymmetric) in order to compartmentalize an organ's interior by having an apical membrane facing an “outside” lumen and a basolateral membrane facing neighboring cells and the basal lamina. These two distinct membrane domains are separated by intercellular junctional complexes, called tight junctions, which render the epithelial cell monolayer selectively permeable to solutes and fluid. Differentially organized apical and basolateral membranes account for ability of epithelial tissue to coordinate secretion and/or absorption from appropriate surfaces. Newly synthesized membrane proteins expressed for various functions are packaged into transport vesicles at the trans-Golgi network (TGN) and differentially sorted during translation and folding to target appropriate membranes within this polarized cellular organization.

Basolateral sorting signals are embedded within the sorted protein's primary structure, usually located in the cytoplasmic tail (or cytosol-facing domain) of the cargo proteins. Many such basolateral sorting signals are known. The most common types of signals involved in sorting of basolateral membrane proteins are tyrosine based (NPxY or YxxΦ) or dileucine (D/ExxxLL), or mono-leucine (EExxxL) motifs (x can be any amino acid; Φ is a bulky hydrophobic residue). Accordingly, in some embodiments, the payload nucleic acid further comprises a basolateral sorting signal for targeting a payload protein to the basolateral cell membrane of an epithelial cell (e.g. a colonic epithelial cell). This enables displaying or secreting payload proteins to the blood stream. For example, antibodies, antigenic proteins, and/or immunomodulatory proteins may be secreted into the blood stream of a subject.

Apical sorting signals are also known, and may be based on amino acid sequence, or post-translational modifications involving lipids or carbohydrates. One commonly characterized apical sorting determinant is the glycosyl phosphatidylinositol-anchored protein linker (GPI-AP). N- and O-linked glycosylation have also been shown to serve as sorting signals for many apical proteins. Various viral single-pass transmembrane domains can serve as signals for apical sorting (eg. Hemagglutinin, neuraminidase and the respiratory syncytial virus F protein). Accordingly, in some embodiments, the payload nucleic acid comprises an apical sorting signal for targeting a payload protein to the lumenal cell membrane of an epithelial cell (e.g. a colonic epithelial cell). This enables displaying or secreting payload proteins to the lumen.

When expressed in a vertebrate cell (e.g. a mammalian cell, or a human cell), both soluble proteins and recycled membrane proteins and membrane-anchored proteins will be processed by antigen presentation pathways, e.g. the major histocompatibility (MHC) class I antigen presentation pathway in mammals.

In some embodiments, the payload nucleic acid encodes one or more immunomodulatory proteins, e.g. one or a combination of IL-12, INFg, TNFa, IL-10, IL-8, IL-2, IL-4, 11-15, IL-18, IL1a/b, IL-6, IL-17, CXCL10, CXCL-13, GSMCF, LTa/b, and/or afunctional derivative of any of the foregoing. An example of a functional derivative of IL-12 is described above. In some embodiments, the payload nucleic acid encodes an antibody or antibody fragment or derivative, e.g. for secretion from the eukaryotic cell. In some embodiments, the payload nucleic acid encodes an enzyme (e.g. a lipase, and the like), a blood-clotting protein (e.g. FVIII, FIX, and the like), a hormone (e.g. HGH, insulin, and the like), receptors (e.g. low-density lipoprotein receptor and the like), or a therapeutic protein. In some embodiments, the payload nucleic acid encodes RNA (e.g. RNAi, siRNA, and the like). In some embodiments, the payload nucleic acid encodes one or more protein components of a pathogen. In some embodiments, the payload nucleic acid encodes an antigen that is specific for or associated with a pathology (e.g. cancer, a toxin, or any other foreign or pathologically relevant molecule). In some embodiments, the payload nucleic acid encodes one or more cancer antigenic peptides, cancer-specific antigens, or cancer-associated antigens. In some embodiments, the payload nucleic acid encodes one of the foregoing payloads. In some embodiments, the payload nucleic acid encodes two or more of the foregoing payloads.

When the payload nucleic acid encodes one or more components of a pathogen, the system may function as a DNA vaccine against that pathogen, causing an adaptive immune response in a subject (e.g. a mammalian subject or a human subject) that is administered the vaccine. The pathogen may be any pathogen. In some embodiments, the pathogen is a virus. In some embodiments, the pathogen is a bacteria. In some embodiments, the pathogen is a parasite. In addition to encoding one or more components of a pathogen, the payload nucleic acid may further encode one or more immunomodulatory proteins, or any other payload. In some embodiments, the payload nucleic acid is codon-optimized for expression in the subject (e.g. mammalian subject, or a human subject).

In some embodiments, the pathogen is a virus. In some of these embodiments, the component of the virus encoded by the payload nucleic acid is a viral coat protein. In some of these embodiments, the component of the virus encoded by the payload nucleic acid is a viral fusion protein, which is responsible for virus-cell fusion and thus virus entry into a cell, or an extracellular domain thereof. In some embodiments, the viral fusion protein is Class I. In some embodiments, the viral fusion protein is Class II. In some embodiments, the viral fusion protein is Class III.

In some embodiments, the virus is a coronavirus. In such embodiments, the payload nucleic acid may encode one or a combination of a coronavirus spike protein (or an antigenic fragment or derivative thereof), a coronavirus matrix protein (also called coronavirus membrane glycoprotein) (or an antigenic fragment or derivative thereof), coronavirus envelope protein (or an antigenic fragment or derivative thereof), and/or a coronavirus nucleocapsid protein (or an antigenic fragment or derivative thereof). Each of the derivative(s) may be at least 80% identical to its respective wildtype reference sequence, including one or a combination of insertions, deletions and/or substitutions. In some embodiments, each derivative may be at least 85% identical to its reference sequence. In some embodiments, each derivative may be at least 90% identical to its reference sequence. In some embodiments, each derivative may be at least 95% identical to the reference sequence. In some embodiments, each derivative may be at least 98% identical to the reference sequence. In some embodiments, each derivative may be at least 99% identical to the reference sequence. In some embodiments, the derivative(s) are 80, 81, 82, 83, 84, 85, 86, 87, 88, 89, 90, 91, 92, 93, 94, 95, 96, 97, 98, or 99% identical to their respective reference sequence(s). In some embodiments, at least 50% of the substitutions are conservative substitutions. In some embodiments, at least 75% of the substitutions are conservative substitutions. In some embodiments, at least 90% of the substitutions are conservative substitutions. In some embodiments, all of the substitutions are conservative substitutions. In some embodiments, each derivative consists of residues that are either identical or substitutions relative to the reference sequence. In some embodiments, the coronavirus is a betacoronavirus species (e.g. SARS-CoV, SARS-CoV-2, MERS-CoV, or the like). In some embodiments, the coronavirus spike protein has the amino acid sequence set out in SEQ ID NO:46, 52, 53, or 56. In some embodiments, the coronavirus matrix protein has the amino acid sequence set out in SEQ ID NO:47. In some embodiments, the coronavirus nucleocapsid protein has the sequence set out in SEQ ID NO:48.

Coronavirus spike proteins consist of multiple domains: e.g. spike proteins from members of the betacoronvirus genus include S1 domain, S2 domain, a transmembrane domain, and a cytoplasmic domain. A cleavage site separates the S1 domain and the S2 domain. The S1 domain encompasses a Receptor Binding Domain (RBD). The RBD encompasses a Receptor Binding Motif (RBM). The betacoronavirus spike protein forms a trimeric membrane protein, which presents the extracellular S1 and S2 domains for interaction with the mammalian immune cell antibody-generation machinery. Intracellularly expressed spike protein is also processed into smaller peptides for presentation by MHC class I complexes to induce adaptive T cell immunity.

In some embodiments, the payload nucleic acid encodes a protein comprising a coronavirus spike protein or an antigenic fragment or derivative thereof (or a betacoronavirus spike protein or an antigenic fragment or derivative thereof). The signal peptide of the spike protein may be excluded or substituted with a different signal peptide that is functional in eukaryotes (e.g. IgK signal peptide, which provides more efficient secretion in mammalian cells). The transmembrane domain of the spike protein may be excluded or substituted with a different transmembrane domain or a different membrane association domain (e.g. a lipid anchor sequence, such as a GPI-encoding sequence). In embodiments lacking a transmembrane domain or a membrane association domain, the inclusion of a signal peptide will result in secretion. The cytoplasmic domain may be excluded or substituted with a different cytoplasmic domain. The S2 domain may be excluded or trimmed. The S1 domain may be trimmed, retaining the RBD, or may be further trimmed, retaining the RBM. The fragment (e.g. S1, S1+S2, RBD, RBM, and the like), or a derivative thereof, may be fused to a multimerization domain (e.g. a trimerization domain). In some embodiments, the protein encoded by the payload nucleic acid comprises truncated (trimmed) spike protein (or a derivative thereof) lacking the cytoplasmic domain. In some embodiments, the truncated spike protein (or a derivative thereof) is fused to a trimerization domain to replace the cytoplasmic domain. In some embodiments, the truncated spike (e.g. trimmed to remove the transmembrane and cytoplasmic domains), or a derivative thereof, is fused with an extracellular trimerization domain, optionally fused to a transmembrane domain or a membrane association domain (e.g. lipid anchor, such as GPI or the like). In some embodiments, the protein encoded by the payload nucleic acid comprises S1 domain (or a derivative thereof) linked to a Type I transmembrane domain. In some embodiments, the protein encoded by the payload nucleic acid comprises S1 domain (or a derivative thereof) linked to a lipid anchor (e.g. GPI and the like). In some embodiments, the protein encoded by the payload nucleic acid is configured for secretion and comprises S1 domain (or a derivative thereof) with a signal peptide and without a transmembrane or membrane associate domain. In some embodiments, the protein encoded by the payload nucleic acid comprises S1 and S2 domains (of derivatives thereof) linked to a substituted transmembrane domain. In some embodiments, the protein encoded by the payload nucleic acid comprises S1 and S2 domains (or derivatives thereof) linked to a lipid anchor (e.g. GPI). In some embodiments, the protein encoded by the payload nucleic acid is configured for secretion and comprises S1 and S2 domains (or derivatives thereof) with a signal peptide and without a transmembrane or membrane associate domain. In some embodiments, the protein encoded by the payload nucleic acid is configured for secretion and comprises RBD (or a derivative thereof) with a signal peptide and without a transmembrane or membrane associate domain. In some embodiments, the protein encoded by the payload nucleic acid is configured for secretion and comprises RBM with a signal peptide and without a transmembrane or membrane associate domain. In some embodiments, the signal peptide is wildtype coronavirus signal peptide, and in other embodiments, the signal peptide is substituted (e.g. with a signal peptide that is more efficient in mammals, such as IgK signal peptide). In some embodiments, the derivative comprises the wildtype RBM sequence. In some embodiments, the derivative comprises the wildtype RBD sequence. In some embodiments, the S1 domain is a wildtype S1 domain. In some embodiments, the S2 domain is a wildtype S1 domain.

In some embodiments, the coronavirus spike protein (or the fragment thereof) is from wildtype SARS-CoV-2. In some embodiments, the derivative of the coronavirus spike protein (or its fragment) is derived from a wildtype SARS-CoV-2 spike protein or fragment thereof. In some embodiments, the SARS-CoV-2 spike protein has the amino acid sequence set out in SEQ ID NO:52 (without signal peptide) or SEQ ID NO:46 (with signal peptide). In some embodiments, the SARS-CoV-2 spike protein has the amino acid sequence set out in SEQ ID NO:56 (without signal peptide) or SEQ ID NO:53 (with signal peptide). In some embodiments, the RBM has the amino acid sequence set out in SEQ ID NO:50 (SARS-CoV-2 RBD). In some embodiments, the RBM has the amino acid sequence set out in SEQ ID NO:54 (SARS-CoV-2 RBD variant B.1.351). In some embodiments, the S1 domain has the sequence set out in amino acids 13-685 of SEQ ID NO:46. In some embodiments, the S1 domain has the sequence set out in amino acids 13-682 of SEQ ID NO:53. In some embodiments, the RBM has the amino acid sequence set out in SEQ ID NO:51 (SARS-CoV-2 RBM). In some embodiments, the RBM has the amino acid sequence set out in SEQ ID NO:55 (SARS-CoV-2 RBM variant B.1.351). In some embodiments, the S2 domain has the sequence set out in amino acids 686-1273 of SEQ ID NO:46. In some embodiments, the S2 domain has the sequence set out in amino acids 683-1270 of SEQ ID NO:53. In some embodiments, the S2 domain has the sequence set out in amino acids 816-1273 of SEQ ID NO:46 (i.e. S2′ domain). In some embodiments, the S2 domain has the sequence set out in amino acids 813-1270 of SEQ ID NO:53 (i.e. S2′ domain). In some embodiments, the transmembrane domain has the sequence set out in amino acids 1214-1234 of SEQ ID NO:46. In some embodiments, the transmembrane domain has the sequence set out in amino acids 1211-1231 of SEQ ID NO:53. In some embodiments, the cytoplasmic domain has the sequence set out in amino acids 1245-1273 of SEQ ID NO:46. In some embodiments, the cytoplasmic domain has the sequence set out in amino acids 1242-1270 of SEQ ID NO:53.

In some embodiments, the protein encoded by the payload nucleic acid is or comprises a derivative of the coronavirus spike protein or a derivative of an antigenic fragment (e.g. S1, S1+S2, RBD, RBM, and the like) of the spike protein. The derivative may be at least 80% identical to its respective wildtype reference sequence, including one or a combination of insertions, deletions and/or substitutions. In some embodiments, the derivative is at least 85% identical to the reference sequence. In some embodiments, the derivative is at least 90% identical to the reference sequence. In some embodiments, the derivative is at least 95% identical to the reference sequence. In some embodiments, the derivative is at least 98% identical to the reference sequence. In some embodiments, the derivative is at least 99% identical to the reference sequence. In some embodiments, the derivative is 80, 81, 82, 83, 84, 85, 86, 87, 88, 89, 90, 91, 92, 93, 94, 95, 96, 97, 98, or 99% identical to the reference sequence(s). In some embodiments, at least 50% of the substitutions are conservative substitutions. In some embodiments, at least 75% of the substitutions are conservative substitutions. In some embodiments, at least 90% of the substitutions are conservative substitutions. In some embodiments, all of the substitutions are conservative substitutions. In some embodiments, the derivative consists of residues that are identical or substitutions relative to the reference sequence. In some embodiments, the reference sequence(s) is/are from wildtype SARS-CoV-2. In some embodiments, the derivative is from a variant of SARS-CoV-2 capable of causing coronavirus disease (COVID) in humans (e.g. COVID-19), e.g. but without limitation variant B.1.1351 lineage (South Africa), B.1.1.7 lineage (U.K.), or P.1 lineage (Brazil/Japan). In some embodiments, the derivative comprises one, two or three of spike (or spike fragment) mutations N501Y, K417N and/or E484K, optionally further comprising D614G. In some embodiments, the derivative comprises one or more spike (or spike fragment) mutations D80A, D215G, K417N, E484K, N501Y, D614G, A701V, delta 242, delta 243, and/or delta 244, and in some embodiments, the derivative comprises all of these mutations. In some embodiments, the derivative further comprises spike (or spike fragment) mutation L18F. In some embodiments, the variant is B.1.1351 lineage. In some embodiments, the derivative comprises one or more spike (or spike fragment) mutations delta 69/70, delta 144, N501Y, A570D, D614G, and/or P681H, and in some embodiments, the derivative comprises all of these mutations. In some embodiments, the variant is B.1.1.7 lineage. In some embodiments, the derivative comprises one or more spike (or spike fragment) mutations E484K, K417N/T, N501Y, optionally further comprising D614G. In some embodiments, the variant is P.1 lineage. The foregoing amino acid position numbers are based on the wildtype SARS-CoV-2 spike sequence (SEQ ID NO:46) as reference.

When the nucleic acid encodes one or more cancer antigenic peptides, cancer-specific antigens, or cancer-associated antigens, the system may function as a DNA vaccine against cancer, and as such may cause an adaptive immune response in a subject (e.g. a mammalian subject or a human subject) that is administered the vaccine. Various cancer related antigens have been reported (see Cancer Antigenic Peptide Database website). In addition to encoding one or more cancer antigenic peptides, cancer-specific antigens or cancer-associated antigens, the payload nucleic acid may further encode one or more immunomodulatory proteins, or any other payload. The cancer antigenic peptide may be a unique antigen (i.e. resulting from point mutations in genes that are ubiquitously expressed), a shared tumor-specific antigen, a differentiation antigen, or an overexpressed antigen. In some embodiments, the payload nucleic acid encodes a cancer-specific antigen. In some embodiments, the payload nucleic acid encodes a cancer-associated antigen.

In some embodiments, the eukaryotic cell is a cell of a subject (e.g. a mammalian subject or a human subject). In some embodiments, the cell is a colonic cell of the subject. In some embodiments, the cell is a colonic epithelial cell, which are readily colonized by Bifidobacterium spp. (e.g. see Example 1 below), a colonic immune cell, or a cell of the lamina propria. As such, the system may be used for delivery of a payload nucleic acid into colonic epithelial cells, colonic immune cells, and/or cells of the lamina propria of a subject and thus production of a payload encoded by the payload nucleic acid in the colonic epithelial cells, colonic immune cells, and/or cells of the lamina propria. The bacterium may access colon cells via oral administration to the subject, although the colon may alternatively be accessed by other routes (e.g. rectal, such as via suppository). In some embodiments, the payload nucleic acid comprises a basolateral sorting signal for targeting a payload protein to the basolateral cell membrane of the colonic epithelial cell. In some embodiments, the payload nucleic acid comprises an apical sorting signal for targeting a payload protein to the lumenal cell membrane of the colonic epithelial cell.

In some embodiments, the system may be formulated as a pharmaceutical composition further comprising one or more pharmaceutically acceptable excipients. Non-limiting examples of suitable excipients include any suitable buffers, stabilizing agents, salts, antioxidants, complexing agents, tonicity agents, cryoprotectants, lyoprotectants, suspending agents, emulsifying agents, antimicrobial agents, preservatives, chelating agents, binding agents, surfactants, wetting agents, non-aqueous vehicles such as fixed oils, or polymers for sustained or controlled release. See, for example, Berge et al. 1977 (J. Pharm Sci. 66:1-19), or Remington—The Science. and Practice of Pharmacy, 21st edition (Gennaro et al. editors. Lippincott Williams & Wilkins Philadelphia). In some embodiments, the pharmaceutical composition comprises a cryo-preservative, e.g. any reagent that can function as a cryo-preservative for freezing live bacterial cells that is suitable as an excipient for administration to humans (e.g. USP-NF or equivalent regulatory designation) or has the potential upon toxicology testing to be applied as an excipient. Non-limiting examples of suitable cryo-preservatives include: trehalose, hydroxyethyl starch (HES/HAES), propylene glycol, simple sugars or disaccharides (e.g. sucrose). In some embodiments, the pharmaceutical composition comprises the tumour-colonizing bacteria in 5-15% sucrose (w/v), 6-14% sucrose (w/v), 7-13% sucrose (w/v), 8-12% sucrose (w/v), or 9-11% sucrose (w/v). In some embodiments, the amount of sucrose is about 9.5 to about 10.5% (w/v). In some embodiments, the amount of sucrose (w/v) is about 5%, about 6%, about 7%, about 8%, about 9%, about 9.2%, about 9.4%, about 9.6%, about 9.8%, about 10%, about 10.2%, about 10.4%, about 10.6%, about 10.8%, about 11%, about 12%, about 13%, about 14%, or about 15%. In some embodiments, the amount of sucrose is about 10% (w/v). In some embodiments, the pharmaceutical composition further comprises a pharmaceutically acceptable buffer (e.g. phosphate buffer), optionally at pH 6-8, e.g. about pH 6.0, 6.2, 6.4, 6.6, 6.8, 7.0, 7.2, 7.4, 7.6, 7.8, 8.0, or about pH 7.2. In some embodiments, the pharmaceutical composition further comprises saline, e.g. phosphate buffered saline (PBS). In some embodiments, the pharmaceutical composition comprises the tumour-colonizing bacteria in a solution of PBS including about 10% sucrose, at a pH of about 7.2-7.4.

In some embodiments, the pharmaceutical composition is formulated for oral administration. In some embodiments, the pharmaceutical composition is formulated as an edible foodstuff (e.g. yoghurt). In some embodiments, the bacterium is lyophilized. Lyophilization methods for probiotic bacteria are well established, enabling the production of a lyophilized drug product with an extensive shelf-life that does not require cold-chain supply logistics.

In some embodiments, the pharmaceutical composition is formulated for rectal administration. For example, but without limitation, the pharmaceutical composition may be formulated as a suppository. Methods and formulations for preparing suppositories are known.

In some embodiments, the pharmaceutical composition is for intravenous administration to the subject. Accordingly, the pharmaceutical composition may be formulated for intravenous injection. Methods for intravenous administration of bacteria into a subject are known, as are suitable formulations for intravenous injection.

In some embodiments, the pharmaceutical composition is for administration (e.g. oral, rectal, intravenous, and the like) to the subject in combination with an immunologic adjuvant. In some embodiments, the pharmaceutical composition is for administration to the subject without an immunologic adjuvant (since the bacterium itself can act as an adjuvant).

In the foregoing, it is mentioned that a plurality of protein and/or RNA payloads may be included in a single plasmid, or may be positioned on separate plasmids. In some embodiments, the separate plasmids (encoding unique sets of payload-encoding sequences) are contained in the same bacterium (i.e. a bacterium is transformed with multiple plasmids that differ in their payload coding sequences). In other embodiments, the separate plasmids are contained in separate bacterium, e.g. a first bacterium and a second bacterium (and optionally a third bacterium, and optionally a fourth bacterium, and optionally further bacteria). As such, a plurality of payload coding sequences may be delivered (for expression in the subject, locally and/or systemically), by administering a combination of bacteria, each bacterium in the combination comprising a plasmid encoding a distinct payload (or distinct set of payloads). For example, but without limitation, in some embodiments one of the bacteria may be configured to deliver one or more antigenic proteins and another of the bacteria may be configured to deliver one or more immunomodulatory proteins (optionally wherein the one or more immunomodulatory proteins is one or a combination of IL-12, INFg, TNFa, IL-10, IL-8, IL-2, IL-4, 11-15, IL-18, IL1a/b, IL-6, IL-17, CXCL10, CXCL-13, GSMCF, LTa/b, and/or a functional derivative of the foregoing). In some embodiments, the different bacteria of the combination may be formulated in a single dosage form for co-administration. For example, the first bacterium and the second bacterium may be formulated in a single dosage form. In some embodiments, the different bacteria of the combination may be formulated as separate dosage forms for administration simultaneously or sequentially. For example, the first bacterium and the second bacterium may be formulated as separate dosage forms.

The dose of bacteria to administer to the subject may be any suitable dose. In some embodiments, the dose is 10⁵ to 10¹¹ colony forming units (CFUs), but lower and higher doses are generally suitable, e.g. doses of 10³-10⁴, 10⁴-10⁵, 10⁵-10⁶, 10⁶-10⁷, 10⁷-10⁸, 10⁸-10⁹, 10⁹-10¹⁰, 10¹⁰-10¹¹ and more than 10¹¹ CFUs. In some embodiments, the dose is 10⁵ to 10¹¹ CFUs. In some embodiments, the dose is 10⁸ to 10¹⁰ CFUs. In some embodiments, the dose is about 10⁹ CFUs.

Should it be desired or necessary to end colonization, methods or uses of the system may further comprise subsequent administration to the subject of an antibiotic to which the bacterium is susceptible. The particular antibiotic that the administered bacteria was susceptible to would be known, as would the methods of administering the antibiotic and the dosage of the antibiotic. In some embodiments, the antibiotic is amoxicillin or erythromycin.

In some embodiments, the subject is a mammal. In some embodiments, the subject is a human.

The following is a non-exhaustive list of embodiments:
A1. A system for use in delivery of a payload nucleic acid into colonic cells (e.g. colonic epithelial cells, colonic immune cells, and/or cells of the lamina propria) of a subject and production of a payload encoded by the payload nucleic acid in the colonic cells;
a Bifidobacterium sp. bacterium comprising a plasmid and a transporter nucleic acid
the transporter nucleic acid in operative association with a first promoter and a first terminator configured to express the transporter nucleic acid in the bacterium;
the transporter nucleic acid having a sequence encoding a transporter polypeptide comprising, in an amino-terminal to carboxy-terminal order, a bacterial secretion signal peptide, a DNA-binding domain, and a cell penetrating peptide, the DNA-binding domain configured for association with the plasmid to form a polypeptide-plasmid complex, the bacterial secretion signal peptide configured for secretion of the polypeptide-plasmid complex from the bacterium, and the cell penetrating peptide configured for importing the polypeptide-plasmid complex into a colonic cell (e.g. a colonic epithelial cell, a colonic immune cell, and/or a cell of the lamina propria) of the subject; and
the plasmid comprising a payload nucleic acid encoding a payload protein or a payload ribonucleic acid, the payload nucleic acid in operative association with a second promoter and a second terminator configured to express the payload nucleic acid in the colonic cell, and produce the payload protein or the payload ribonucleic acid.
A2. The system of embodiment A1, wherein the Bifidobacterium sp. bacterium is Bifidobacterium longum.
A3. The system of embodiment A1 or A2, wherein the bacterial secretion signal peptide is an alpha-arabinosidase secretion signal peptide.
A4. The system of embodiment A3, wherein the alpha-arabinosidase secretion signal peptide has sequence SEQ ID NO: 13.
A5. The system of any one of embodiments A1 to A4, wherein the DNA-binding domain has sequence SEQ ID NO: 7.
A6. The system of any one of embodiments A1 to A5, wherein the cell penetrating peptide has sequence SEQ ID NO: 18.
A7. The system of embodiment A1 or A2, wherein the transporter polypeptide has sequence SEQ ID NO: 2.
A8. The system of any one of embodiments A1 to A7, wherein the plasmid further comprises the transporter nucleic acid.
A9. The system of any one of embodiments A1 to A8, wherein the payload nucleic acid comprises a basolateral sorting signal for targeting a payload protein to the basolateral cell membrane of the colonic epithelial cell.
A10. The system of any one of embodiments A1 to A8, wherein the payload nucleic acid comprises an apical sorting signal for targeting a payload protein to the lumenal cell membrane of the colonic epithelial cell.
A11. The system of any one of embodiments A1 to A10, wherein payload protein is a membrane or membrane-associated protein comprising an extracellular domain.
A12. The system of embodiment A11, wherein the membrane or membrane-associated protein is an integral membrane protein.
A13. The system of any one of embodiments A1 to A10, wherein the plasmid further encodes a lipid anchor signal peptide in operative association with the payload nucleic acid to produce the payload protein as a lipid anchored protein.
A12. The system of any one of embodiments A1 to A10, wherein the plasmid further encodes a secretion signal peptide in operative association with the payload nucleic acid to secrete the payload protein.
A13. The system of any one of embodiments A1 to A8, wherein the plasmid is configured to produce the payload protein as an intracellular protein.
A14. The system of any one of embodiments A1 to A13, wherein the payload nucleic acid encodes, alone or in combination with other nucleic acid(s), an antigen from a pathogen, an antigen that is specific for or associated with a pathology, optionally cancer, an immunomodulatory protein, an antibody or antibody fragment or derivative, an enzyme, a receptor, or a therapeutic protein.
A15. The system of embodiment A14, wherein the payload protein comprises: a coronavirus spike protein, matrix protein or nucleocapsid protein; or a betacoronavirus spike protein, matrix protein or nucleocapsid protein.
A16. The system of embodiment A15, wherein the coronavirus spike protein, matrix protein or nucleocapsid protein is SARS-CoV-2 spike protein, matrix protein or nucleocapsid protein.
A17. The system of any one of embodiments A14 to A16, wherein the payload nucleic acid encodes a plurality of payloads, the plurality of payloads comprising a combination of antigenic proteins from the pathogen.
A18. The system of any one of embodiments A1 to A16, wherein the payload nucleic acid encodes a plurality of payloads.
A19. The system of embodiment A17 or A18, wherein the plurality of payloads comprises one or more immunomodulatory proteins, optionally wherein the one or more immunomodulatory proteins is one or a combination of IL-12, INFg, TNFa, IL-10, IL-8, IL-2, IL-4, 11-15, IL-18, IL1a/b, IL-6, IL-17, CXCL10, CXCL-13, GSMCF, LTa/b, and/or afunctional derivative of the foregoing.
A20. The system of any one of embodiments A17 to A19, wherein the payload nucleic acid comprises a plurality of payload coding sequences, wherein the payload nucleic acid is operatively associated with a single promoter and terminator for expression in the colonic cells, and wherein each payload coding sequence is separated by an IRES element.
A21. The system of any one of embodiments A17 to A19, wherein the payload nucleic acid comprises a plurality of payload coding sequences and each payload coding sequence is operatively associated with a separate promoter and terminator for expression in the colonic cells.
A22. The system of any one of embodiments A1 to A21, wherein the system is for use in delivery of the payload nucleic acid into colonic epithelial cells and/or colonic immune cells of a subject and production of the payload encoded by the payload nucleic acid in the colonic epithelial cells and/or colonic immune cells, wherein the cell penetrating peptide configured for importing the polypeptide-plasmid complex into the colonic epithelial cell and/or the colonic immune cell, and wherein the second promoter and the second terminator are configured to express the payload nucleic acid in the colonic epithelial cell and/or the colonic immune cell.
A23. The system of any one of embodiments A1 to A22, wherein the system is formulated as a pharmaceutical composition further comprising a pharmaceutically acceptable excipient.
A24. The system of embodiment A23, wherein the pharmaceutical composition is for oral administration.
A25. The system of embodiment A23 or A24, wherein the pharmaceutical composition is for administration in combination with an immunologic adjuvant.
A26. The system of any one of embodiments A1 to A25, wherein the bacterium is lyophilized.
A27. The system of any one of embodiments A1 to A26, wherein the system is for administration to the subject in a dose of 10⁵ to 10¹¹ colony forming units (CFUs), or optionally at a dose of 10⁸ to 10¹⁰ CFUs.
A28. The system of any one of embodiments A1 to A27, wherein the bacterium is a first bacterium and is for administration in combination with a second bacterium as defined in embodiment A1, wherein the payload protein or the payload ribonucleic acid encoded by the payload nucleic acid of the first bacterium is distinct from the payload protein or the payload ribonucleic acid encoded by the payload nucleic acid of the second bacterium.
A29. The system of embodiment A28, wherein the first bacterium and the second bacterium are formulated together in a single dosage form for co-administration.
A30. The system of embodiment A28, wherein the first bacterium and the second bacterium are formulated as separate dosage forms.
B1. A method for delivering a payload nucleic acid into colonic cells (e.g. colonic epithelial cells, colonic immune cells, and/or cells of the lamina propria) of a subject and causing the cells to produce a payload encoded by the payload nucleic acid,
the method comprising administering to the subject a Bifidobacterium sp. bacterium comprising a plasmid and a transporter nucleic acid such that the bacterium colonizes the colon of the subject;
the transporter nucleic acid is in operative association with a first promoter and a first terminator configured to express the transporter nucleic acid in the bacterium;
the transporter nucleic acid encoding a transporter polypeptide comprising, in an amino-terminal to carboxy-terminal order, a bacterial secretion signal peptide, a DNA-binding domain, and a cell penetrating peptide, the DNA-binding domain configured for association with the plasmid to form a polypeptide-plasmid complex, the bacterial secretion signal peptide configured for secretion of the polypeptide-plasmid complex from the bacterium, and the cell penetrating peptide configured for importing the polypeptide-plasmid complex into a colonic cell (e.g. a colonic epithelial cell, a colonic immune cell, and/or a cell of the lamina propria) of the subject; and
the plasmid comprising a payload nucleic acid having a sequence encoding a payload protein or a payload ribonucleic acid, the payload nucleic acid in operative association with a second promoter and a second terminator configured to express the payload nucleic acid in the colonic cell and to produce the payload protein or the payload ribonucleic acid.
B2. The method of embodiment B1, wherein the bacterium is as defined in the system of any one of embodiments A1 to A22.
B3. The method of embodiment B1 or B2, wherein the bacterium is administered in a pharmaceutical composition further comprising a pharmaceutically acceptable excipient.
B4. The method of embodiment B3, wherein the pharmaceutical composition is orally administered.
B5. The method of embodiment B3 or B4, wherein the pharmaceutical composition is administered in combination with an immunologic adjuvant.
B6. The method of any one of embodiments B3 to B5, wherein the bacterium is lyophilized in the pharmaceutical composition.
B7. The method of any one of embodiments B3 to B6, wherein the pharmaceutical composition is administered to the subject in a dose of 10⁵ to 10¹¹ colony forming units (CFUs), or optionally at a dose of 10⁸ to 10¹⁰ CFUs.
B8. The method of any one of embodiments B1 to B7, wherein the bacterium is a first bacterium and is administered in combination with a second bacterium as defined in embodiment B1 or embodiment B2, wherein the payload protein or the payload ribonucleic acid encoded by the payload nucleic acid of the first bacterium is distinct from the payload protein or the payload ribonucleic acid encoded by the payload nucleic acid of the second bacterium.
B9. The method of embodiment B8, wherein the first bacterium and the second bacterium are formulated in a single dosage form for co-administration.
B10. The method of embodiment B8, wherein the first bacterium and the second bacterium are formulated as separate dosage forms.
C1. A DNA vaccine comprising:
a Bifidobacterium sp. bacterium comprising a plasmid and a transporter nucleic acid
the transporter nucleic acid in operative association with a first promoter and a first terminator configured to express the transporter nucleic acid in the bacterium;
the transporter nucleic acid encoding a transporter polypeptide comprising, in an amino-terminal to carboxy-terminal order, a bacterial secretion signal peptide, a DNA-binding domain, and a cell penetrating peptide, the DNA-binding domain configured for association with the plasmid to form a polypeptide-plasmid complex, the bacterial secretion signal peptide configured for secretion of the polypeptide-plasmid complex from the bacterium, and the cell penetrating peptide configured for importing the polypeptide-plasmid complex into a cell of a subject; and
the plasmid comprising a payload nucleic acid encoding a payload protein, the payload nucleic acid in operative association with a second promoter and a second terminator configured to express the payload gene in the cell and produce the payload protein, wherein the payload protein is a component of a pathogen or wherein the payload protein comprises an antigen that is specific for or associated with a pathology, optionally wherein the pathology is a cancer.
C2. The DNA vaccine of embodiment C1, wherein the DNA vaccine causes an adaptive immune response in the subject against the pathogen or the pathology following administration of the DNA vaccine to the subject.
C3. The DNA vaccine of embodiment C1 or C2, wherein the Bifidobacterium sp. bacterium is Bifidobacterium longum.
C4. The DNA vaccine of any one of embodiments C1 to C3, wherein the bacterial secretion signal peptide is an alpha-arabinosidase secretion signal peptide.
C5. The DNA vaccine of embodiment C4, wherein the alpha-arabinosidase secretion signal peptide has sequence SEQ ID NO: 13.
C6. The DNA vaccine of any one of embodiments C1 to C5, wherein the DNA-binding domain has sequence SEQ ID NO: 7.
C7. The DNA vaccine of any one of embodiments C1 to C6, wherein the cell penetrating peptide has sequence SEQ ID NO: 18.
C8. The DNA vaccine of any one of embodiments C1 to C3, wherein the transporter polypeptide has sequence SEQ ID NO: 2.
C9. The DNA vaccine of any one of embodiments C1 to C8, wherein the plasmid further comprises the transporter nucleic acid.
C10. The DNA vaccine of any one of embodiments C1 to C9, wherein the pathogen is a virus, a bacteria or a parasite.
C11. The DNA vaccine of embodiment C10, wherein the pathogen is a virus.
C12. The DNA vaccine of embodiment C11, wherein the virus is a coronavirus, optionally a betacoronavirus, optionally SARS-CoV-2 (wildtype or variant).
C13. The DNA vaccine of embodiment C12, wherein the payload protein comprises a spike protein or an antigenic fragment thereof, a matrix protein or an antigenic fragment thereof, or a nucleocapsid protein or an antigenic fragment thereof.
C14. The DNA vaccine of embodiment C12, wherein the payload protein comprises a spike protein or an antigenic fragment or derivative thereof, a matrix protein or an antigenic fragment or derivative thereof, or a nucleocapsid protein or an antigenic fragment or derivative thereof.
C15. The DNA vaccine of embodiment C12, wherein the payload protein comprises a spike protein fragment or a derivative that is at least 80% identical to a wildtype sequence, wherein the spike protein fragment comprises a receptor binding domain (RBD) of the spike protein.
C16. The DNA vaccine of embodiment C15, wherein the payload protein comprises the amino acid sequence set out in SEQ ID NO:51 or 55, and the spike protein fragment is a derivative that is at least 80% identical to amino acids 13-685 of SEQ ID NO:46.
C17. The DNA vaccine of embodiment C15, wherein the payload protein comprises the amino acid sequence set out in any one of SEQ ID NOs:46, and 53-56.
C18. The DNA vaccine of embodiment C15, wherein the payload protein comprises amino acids 13-685 of SEQ ID NO:46 or amino acids 13-682 of SEQ ID NO:53, or optionally comprises amino acids 13-1273 of SEQ ID NO:46 or amino acids 13-1270 of SEQ ID NO:53.
C19. The DNA vaccine of any one of embodiments C13 to C18, wherein the payload nucleic acid encodes a plurality of payloads comprising a combination of: a spike protein or an antigenic fragment thereof, a matrix protein or an antigenic fragment thereof, and/or a nucleocapsid protein or an antigenic fragment thereof.
C20. The DNA vaccine of any one of embodiments C1 to C18, wherein the payload nucleic acid encodes a plurality of payloads.
C21. The DNA vaccine of embodiment C20, wherein the plurality of payloads comprises a combination of antigenic proteins from the pathogen.
C22. The DNA vaccine of any one of embodiments C19 to C21, wherein the plurality of payloads comprises one or more immunomodulatory proteins, optionally wherein the one or more immunomodulatory proteins is one or a combination of IL-12, INFg, TNFa, IL-10, IL-8, IL-2, IL-4, 11-15, IL-18, IL1a/b, IL-6, IL-17, CXCL10, CXCL-13, GSMCF, LTa/b, and/or a functional derivative of the foregoing.
C23. The DNA vaccine of any one of embodiments C19 to C22, wherein the payload nucleic acid comprises a plurality of payload coding sequences, wherein the payload nucleic acid is operatively associated with a single promoter and terminator for expression in the cell of the subject, and wherein each payload coding sequence is separated by an IRES element.
C24. The DNA vaccine of any one of embodiments C19 to C22, wherein the payload nucleic acid comprises a plurality of payload coding sequences and each payload coding sequence is operatively associated with a separate promoter and terminator for expression in the cell of the subject.
C25. The DNA vaccine of any one of embodiments C1 to C24, wherein a payload protein is a membrane or membrane-associated protein comprising an extracellular domain.
C26. The DNA vaccine of embodiment C25, wherein the membrane or membrane-associated protein is an integral membrane protein.
C27. The DNA vaccine of any one of embodiments C1 to C26, wherein the plasmid further encodes a lipid anchor signal peptide in operative association with a payload nucleic acid to produce a payload protein as a lipid anchored protein.
C28. The DNA vaccine of any one of embodiments C1 to C27, wherein the plasmid further encodes a secretion signal peptide in operative association with a payload nucleic acid to secrete a payload protein.
C29. The DNA vaccine of any one of embodiments C1 to C28, wherein the plasmid is configured to produce a payload protein as an intracellular protein.
C30. The DNA vaccine of any one of embodiments C1 to C29, wherein the cell of the subject is a colonic cell (e.g. a colonic epithelial cell, a colonic immune cell, and/or a cell of the lamina propria), optionally wherein the cell of the subject is a colonic epithelial cell and/or a colonic immune cell.
C31. The DNA vaccine of embodiment C30, wherein the payload nucleic acid comprises a basolateral sorting signal for targeting a payload protein to the basolateral cell membrane of the colonic epithelial cell.
C32. The DNA vaccine of embodiment C30 or C31, wherein the payload nucleic acid comprises an apical sorting signal for targeting a payload protein to the lumenal cell membrane of the colonic epithelial cell.
C33. The DNA vaccine of any one of embodiments C1 to C32, wherein the DNA vaccine is formulated as a pharmaceutical composition further comprising a pharmaceutically acceptable excipient.
C34. The DNA vaccine of embodiment C33, wherein the pharmaceutical composition is for oral administration.
C35. The DNA vaccine of embodiment C33 or C34, wherein the pharmaceutical composition is for administration in combination with an immunologic adjuvant.
C36. The DNA vaccine of any one of embodiments C1 to C35, wherein the bacterium is lyophilized.
C37. The DNA vaccine of any one of embodiments C1 to C36, wherein the DNA vaccine is for administration to the subject in a dose of 10⁵ to 10¹¹ colony forming units (CFUs), or optionally 10⁸ to 10¹⁰ CFUs.
C38. The DNA vaccine of any one of embodiments C1 to C37, wherein the bacterium is a first bacterium and is for administration in combination with a second bacterium as defined in embodiment A1 or C1, wherein the payload protein encoded by the payload nucleic acid of the first bacterium is distinct from the payload protein or the payload ribonucleic acid encoded by the payload nucleic acid of the second bacterium.
C39. The DNA vaccine of embodiment C38, wherein the first bacterium and the second bacterium are formulated in a single dosage form for co-administration.
C40. The DNA vaccine of embodiment C38, wherein the first bacterium and the second bacterium are formulated as separate dosage forms.
C41. The DNA vaccine of any one of embodiments C38 to C40, wherein the payload protein or the payload ribonucleic acid encoded by the payload nucleic acid of the second bacterium comprises one or more immunomodulatory proteins, optionally wherein the one or more immunomodulatory proteins is one or a combination of IL-12, INFg, TNFa, IL-10, IL-8, IL-2, IL-4, 11-15, IL-18, IL1a/b, IL-6, IL-17, CXCL10, CXCL-13, GSMCF, LTa/b, and/or a functional derivative of the foregoing.
D1. A method of vaccinating a subject against a pathogen, the method comprising administering to the subject the DNA vaccine of any one of embodiments C1 to C32, wherein the payload nucleic acid encodes one or more components of the pathogen.
D2. A method of vaccinating a subject against a coronavirus, the method comprising administering to the subject the DNA vaccine of any one of embodiments C12 to C19.
D3. The method of embodiment D2, wherein the coronavirus is a betacoronavirus, optionally SARS-CoV-2.
D4. A method of vaccinating a subject against a pathology, the method comprising administering to the subject the DNA vaccine of any one of embodiments C1 to C32, wherein the payload nucleic acid encodes an antigen that is specific for or associated with the pathology, optionally wherein the pathology is a cancer.
D5. The method of embodiment D4, wherein the bacterium is administered in a pharmaceutical composition further comprising a pharmaceutically acceptable excipient.
D6. The method of embodiment D5, wherein the pharmaceutical composition is orally administered.
D7. The method of embodiment D5 or D6, wherein the pharmaceutical composition is administered in combination with an immunologic adjuvant.
D8. The method of any one of embodiments D5 to D7, wherein the bacterium is lyophilized in the pharmaceutical composition.
D9. The method of any one of embodiments D5 to D8, wherein the pharmaceutical composition is administered to the subject in a dose of 10⁵ to 10¹¹ colony forming units (CFUs), or optionally at a dose of 10⁸ to 10¹⁰ CFUs.
D10. The method of any one of embodiments D1 to D9, wherein the bacterium is a first bacterium and is administered in combination with a second bacterium as defined in embodiment A1 or C1, wherein the payload protein encoded by the payload nucleic acid of the first bacterium is distinct from the payload protein or the payload ribonucleic acid encoded by the payload nucleic acid of the second bacterium.
D11. The method of embodiment D10, wherein the first bacterium and the second bacterium are formulated together in a single dosage form for co-administration.
D12. The method of embodiment D10, wherein the first bacterium and the second bacterium are formulated as separate dosage forms.
D13. The method of any one of embodiments D10 to D12, wherein the payload protein or the payload ribonucleic acid encoded by the payload nucleic acid of the second bacterium comprises one or more immunomodulatory proteins, optionally wherein the one or more immunomodulatory proteins is one or a combination of IL-12, INFg, TNFa, IL-10, IL-8, IL-2, IL-4, 11-15, IL-18, IL1a/b, IL-6, IL-17, CXCL10, CXCL-13, GSMCF, LTa/b, and/or a functional derivative of the foregoing.

### III. Sequences

Table 1 lists various sequences referenced in this application.

TABLE 1
Sequences

| SEQ | | |
|---|---|---|
| ID | | Other identifying |
| NO | Sequence (amino acid or DNA 5′ to 3′) | information |
| 1 | [purposely left blank] | |
| 2 | MTLTGTLRKAFATTLAAAMLIGTLAGCSSAAYNKSDLVSKIAQKSNLT | Transporter polypeptide |
| | KAQAEAAVNAFQDVFVEAMKSGEGLKLTGLESAERVKRPARTGRNPRT | HTP (artificial sequence) |
| | GEQIDIPASYGVRISAGSLLKKAVTEYGRKKRRQRRR | |
| 3 | MCHQQLVISWFSLVFLASPLVAIWELKKDVYVVELDWYPDAPGEMVVL | M-IL-12 (artificial) |
| | TCDTPEEDGITWTLDQSSEVLGSGKTLTIQVKEFGDAGQYTCHKGGEV | Hu-IL-12 beta (p40) |
| | LSHSLLLLHKKEDGIWSTDILKDQKEPKNKTFLRCEAKNYSGRFTCWW | 1-328 aa |
| | LTTISTDLTFSVKSSRGSSDPQGVTCGAATLSAERVRGDNKEYEYSVE | 1x(G6S) Linker |
| | CQEDSACPAAEESLPIEVMVDAVHKLKYENYTSSFFIRDIIKPDPPKN | 329-335 aa |
| | LQLKPLKNSRQVEVSWEYPDTWSTPHSYFSLTFCVQVQGKSKREKKDR | Hu-IL-12 alpha (p35) |
| | VFTDKTSATVICRKNASISVRAQDRYYSSSWSEWASVPCSGGGGGGSR | 336-532 aa |
| | NLPVATPDPGMFPCLHHSQNLLRAVSNMLQKARQTLEFYPCTSEEIDH | 2x(G6S) Linker |
| | EDITKDKTSTVEACLPLELTKNESCLNSRETSFITNGSCLASRKTSFM | 533-546 aa |
| | MALCLSSIYEDLKMYQVEFKTMNAKLLMDPKRQIFLDQNMLAVIDELM | INSR-TM Domain |
| | QALNFNSETVPQKSSLEEPDFYKTKIKLCILLHAFRIRAVTIDRVMSY | 547-577 aa |
| | LNASGGGGGGSGGGGGGSIIIGPLIFVFLFSVVIGSIYLELRKRQPDG | |
| | P | |
| 4 | gaaaacaatttggagaacctgaccattggcgttttcgccaggacggcc | MerR DNA binding |
| | ggggtcaatgtggagaccatccggttctatcagcgcaagggcttgctc | domain (source: |
| | ccggaaccggacaagccttacggcagcattcgccgctatggcgagacg | Bifidobacterium longum) |
| | gatgtaacgcgggtgcgcttcgtgaaatcagcccagcggttgggcttc | |
| | agcctggatgagatcgccgagctgctgcggctggaggatggcacccat | |
| | tgcgaggaagccagcagcctggccgagcacaagctcaaggacgtgcgc | |
| | gagaggatggctgacctggcgcgcatggaggccgtgctgtctgatttg | |
| | gtgtgcgcctgccatgcgcgaagggggaacgtttcctgcccgctgatc | |
| | gcgtcactacagggtggagcaagcttggcaggttcggctatgcct | |
| 5 | ENNLENLTIGVFARTAGVNVETIRFYQRKGLLPEPDKPYGSIRRYGET | MerR DNA binding |
| | DVTRVRFVKSAQRLGFSLDEIAELLRLEDGTHCEEASSLAEHKLKDVR | domain (source: |
| | ERMADLARMEAVLSDLVCACHARRGNVSCPLIASLQGGASLAGSAMP | Bifidobacterium longum) |
| 6 | gcatacaacaagtctgacctcgtttcgaagatcgcccagaagtccaac | Hu DNA binding domain |
| | ctgaccaaggctcaggccgaggctgctgttaacgccttccaggatgtg | (source: Bifidobacterium |
| | ttcgtcgaggctatgaagtccggcgaaggcctgaagctcaccggcctg | longum) |
| | ttctccgctgagcgcgtcaagcgcccggctcgcaccggccgcaacccg | |
| | cgcactggcgagcagattgacattccggcttcctacggcgttcgtatc | |
| | tccgctggctccctgctgaagaaggccgtcaccgag | |
| 7 | AYNKSDLVSKIAQKSNLTKAQAEAAVNAFQDVFVEAMKSGEGLKLTGL | Hu DNA binding domain |
| | FSAERVKRPARTGRNPRTGEQIDIPASYGVRISAGSLLKKAVTE | (source: Bifidobacterium |
| | | longum) |
| 8 | gaaaaactgcgcaacggcagcggcgatccgggcaaaaaaaaacagcat | Zinc finger DNA binding |
| | gcgtgcccggaatgcggcaaaagctttagccagagcagcgatctgcag | domain (artificial) |
| | cgccatcagcgcacccataccggcgaaaaaccgtataaatgcccggaa | |
| | tgcggcaaaagctttagccgcagcgatgaactgcagcgccatcagcgc | |
| | acccataccggcgaaaaaccgtataaatgcccggaatgcggcaaaagc | |
| | tttagccgcagcgatcatctgagccgccatcagcgcacccatcagaac | |
| | aaaaaa | |
| 9 | EKLRNGSGDPGKKKQHACPECGKSFSQSSDLQRHQRTHTGEKPYKCPE | Zinc finger DNA binding |
| | CGKSFSRSDELQRHQRTHTGEKPYKCPECGKSFSRSDHLSRHQRTHQN | domain (artificial) |
| | KK | |
| 10 | aagcttgtcgacctgcagccttggcgcttgactccgtacatgagtacg | MerR DNA binding |
| | gaagtaaggttacgctatccttggctgcagaagctt | domain recognition site |
| | | (source: Bifidobacterium |
| | | longum) |
| 11 | aagcttttttttttttggggcggctttttttttttaagctt | Zinc finger DNA binding |
| | | domain recognition site |
| | | (artificial) |
| 12 | atgaccctgaccggcaccctgcgcaaggccttcgccaccaccctggcc | Alpha arabinisodiase |
| | gccgccatgctgatcggcaccctggccggctgctcctccgcc | secretion signal peptide |
| | | (source: Bifidobacterium |
| | | longum) |
| 13 | MTLTGTLRKAFATTLAAAMLIGTLAGCSSA | Alpha arabinisodiase |
| | | secretion signal peptide |
| | | (source: Bifidobacterium |
| | | longum) |
| 14 | atgaaacatcggaaacccgcaccggcctggcataggctggggctgaag | Alpha amylase secretion |
| | attagcaagaaagtggtggtcggcatcaccgccgcggcgaccgccttc | signal peptide with |
| | ggcggactggcaatc | C-terminal deletion |
| | | (source: Bifidobacterium |
| | | longum) |
| 15 | MKHRKPAPAWHRLGLKISKKVVVGITAAATAFGGLAI | Alpha amylase secretion |
| | | signal peptide with |
| | | C-terminal deletion |
| | | (source: Bifidobacterium |
| | | longum) |
| 16 | atgaaacatcggaaacccgcaccggcctggcataggctggggctgaag | Alpha amylase secretion |
| | attagcaagaaagtggtggtcggcatcaccgccgcggcgaccgccttc | signal peptide |
| | ggcggactggcaatcgccagcaccgcagcacaggccagcacc | (source: Bifidobacterium |
| | | longum) |
| 17 | MKHRKPAPAWHRLGLKISKKVVVGITAAATAFGGLAIASTAAQAST | Alpha amylase secretion |
| | | signal peptide |
| | | (source: Bifidobacterium |
| | | longum) |
| 18 | YGRKKRRQRRR | TAT CPP (HIV-1) |
| 19 | RQIKIWFQNRRMKWKK | Antp CPP (Antennapedia |
| | | Drosophilamelanogaster) |
| 20 | TRQARRNRRRRWRERQF | Rev CPP (Human |
| | | immunodeficiency virus) |
| 21 | NAKTRRHERRRKLAIER | VP22 CPP (Human |
| | | immunodeficiency virus) |
| 22 | GALFLGFLGAAGSTMGAWSQPKKKRKV | P-beta MPG CPP (Simian |
| | | Virus 40) |
| 23 | GWTLNSAGYLLGKINLKALAALAKKIL | Transportan (Galanin- |
| | | mastoparan) CPP |
| | | (synthetic) |
| 24 | KETWWETWWTEWSQPKKKR(R/K)V | Pep-1 CPP (Simian Virus |
| | | 40) |
| 25 | GRKKRRQRRR | TAT CPP (HIV-1) |
| 26 | RRIPNRRPRR | HRSV CPP (artificial) |
| 27 | RLRWR | AIP6 CPP (artificial) |
| 28 | MVRRELVTLRIRRACGPPRVRV | ARF(1-22) CPP |
| | | (artificial) |
| 29 | LLIILRRRIRKQAHAHSK | pVEC CPP (artificial) |
| 30 | QLALQLALQALQAALQLA | MAP17 CPP (artificial) |
| 31 | DPKGDPKGVTVTVTVTVTGKGDPKPD | VT5 CPP (artificial) |
| 32 | RRIRPRPPRLPRPRPRPLPFPRPG | Bac7 CPP (artificial) |
| 33 | PPRPPRPPR | (PPR)n CPP (artificial) |
| 34 | HGLASTLTRWAHYNALIRAF | gH625 CPP (HSV-1) |
| 35 | WEAALAEALAEALAEHLAEALAEALEALAA | GALA CPP (artificial) |
| 36 | GLFEAIEGFIENGWEGMIDGWYGC | INF7 CPP (influenza) |
| 37 | CSIPPEVKENKPFVYLI | C105Y CPP (artificial) |
| 38 | PFVYLI | PFVYLI CPP (artificial) |
| 39 | SDLWEMMMVSLACQY | Pep-7 CPP (artificial) |
| 40 | GLWRALWRLLRSLWRLLWRA | CADY CPP (artificial) |
| 41 | KLALKLALKALKAALKLA | MAP CPP (artificial) |
| 42 | RRWWRRWRR | R6W3 CPP (artificial) |
| 43 | atgtgagcaaaaggccagcaaaaggccagggaccgtaaaaaggccgcg | DNA sequence of |
| | ttgctggcgtttttccataggctccgcccccctgacgagcatcacaaa | pFRG3.5-CMV-GLuc |
| | aatcgacgctcaagtcagaggtggcgaaacccgacaggactataaaga | plasmid (artificial), |
| | taccaggcgtttccccctggaagctccctcgtgcgctctcctgttccg | payload secreted Gaussia |
| | accctgccgcttaccggatacctgtccgcctttctcccttcgggaagc | luciferase |
| | gtggcgctttctcatagctcacgctgtaggtatctcagttcggtgtag | E. coli Origin of |
| | gtcgttcgctccaagctgggctgtgtgcacgaaccccccgttcagccc | Replication |
| | gaccgctgcgccttatccggtaactatcgtcttgagtccaacccggta | 1-692 bp |
| | agacacgacttatcgccactggcagcagccactggtaacaggattagc | Spectinomycin Resistance |
| | agagcgaggtatgtaggcggtgctacagagttcttgaagtggtggcct | Cassette 699-1734 bp |
| | aactacggctacactagaagaacagtatttggtatctgcgctctgctg | CMV Enhancer/Promoter |
| | aagccagttaccttcggaaaaagagttggtagctcttgatccggcaaa | sequence 1741-2467 bp |
| | caaaccaccgctggtagcggtggtttttttgtttgcaagcagcagatt | [CMV Enhancer 1791- |
| | acgcgcagaaaaaaaggatctcaagaagatcctttgatcttttctacg | 2165 bp] |
| | ggtttttttggggcggctttgaattcttttttttggggcggctttttt | [CMV Promoter 2166- |
| | tttatgcgctcacgcaactggtccagaaccttgaccgaacgcagcggt | 2385 bp] |
| | ggtaacggcgcagtggcggttttcatggcttgttatgactgttttttt | [CMV 5′UTR 2386- |
| | ggggtacagtctatgcctcgggcatccaagcagcaagcgcgttacgcc | 2457 bp] |
| | gtgggtcgatgtttgatgttatggagcagcaacgatgttacgcagcag | Gaussia Luciferase 2478- |
| | ggcagtcgccctaaaacaaagttaaacatcatgagggaagcggtgatc | 3035 bp |
| | gccgaagtatcgactcaactatcagaggtagttggcgtcatcgagcgc | HSV-TK PolyA Signal |
| | catctcgaaccgacgttgctggccgtacatttgtacggctccgcagtg | 3036-3356 bp |
| | gatggcggcctgaagccacacagtgatattgatttgctggttacggtg | Bacterial Rb Promoter |
| | accgtaaggcttgatgaaacaacgcggcgagctttgatcaacgacctt | 3363-3436 bp |
| | ttggaaacttcggcttcccctggagagagcgagattctccgcgctgta | HTP 3437-3838 bp |
| | gaagtcaccattgttgtgcacgacgacatcattccgtggcgttatcca | [Secretion Signal 3437- |
| | gctaagcgcgaactgcaatttggagaatggcagcgcaatgacattctt | 3526 bp] |
| | gcaggtatcttcgagccagccacgatcgacattgatctggctatcttg | [DNA Binding Domain |
| | ctgacaaaagcaagagaacatagcgttgccttggtaggtccagcggcg | 3527-3802 bp] |
| | gaggaactctttgatccggttcctgaacaggatctatttgaggcgcta | [CPP 3803-3838 bp] |
| | aatgaaaccttaacgctatggaactcgccgcccgactgggctggcgat | SynS Terminator 3839- |
| | gagcgaaatgtagtgcttacgttgtcccgcatttggtacagcgcagta | 3880 bp |
| | accggcaaaatcgcgccgaaggatgtcgctgccgactgggcaatggag | Bif Origin of Replication |
| | cgcctgccggcccagtatcagcccgtcatacttgaagctagacaggct | 3887-5714 bp |
| | tatcttggacaagaagaagatcgcttggcctcgcgcgcagatcagttg | |
| | gaagaatttgtccactacgtgaaaggcgagatcaccaaggtagtcggc | |
| | aaataaggtaccgttaggcgttttcgcgatgtacgggccagatatacg | |
| | cgttgacattgattattgactagttattaatagtaatcaattacgggg | |
| | tcattagttcatagcccatatatggagttccgcgttacataacttacg | |
| | gtaaatggcccgcctggctgaccgcccaacgacccccgcccattgacg | |
| | tcaataatgacgtatgttcccatagtaacgccaatagggactttccat | |
| | tgacgtcaatgggtggagtatttacggtaaactgcccacttggcagta | |
| | catcaagtgtatcatatgccaagtacgccccctattgacgtcaatgac | |
| | ggtaaatggcccgcctggcattatgcccagtacatgaccttatgggac | |
| | tttcctacttggcagtacatctacgtattagtcatcgctattaccatg | |
| | gtgatgcggttttggcagtacatcaatgggcgtggatagcggtttgac | |
| | tcacggggatttccaagtctccaccccattgacgtcaatgggagtttg | |
| | ttttggcaccaaaatcaacgggactttccaaaatgtcgtaacaactcc | |
| | gccccattgacgcaaatgggcggtaggcgtgtacggtgggaggtctat | |
| | ataagcagagctcgtttagtgaaccgtcagatcgcctggagacgccat | |
| | ccacgctgttttgacctccatagaagacaccgggaccgatccagcctc | |
| | cggactctagaggatcgaagctagccaccatgggagtcaaagttctgt | |
| | ttgccctgatctgcatcgctgtggccgaggccaagcccaccgagaaca | |
| | acgaagacttcaacatcgtggccgtggccagcaacttcgcgaccacgg | |
| | atctcgatgctgaccgcgggaagttgcccggcaagaagctgccgctgg | |
| | aggtgctcaaagagatggaagccaatgcccggaaagctggctgcacca | |
| | ggggctgtctgatctgcctgtcccacatcaagtgcacgcccaagatga | |
| | agaagttcatcccaggacgctgccacacctacgaaggcgacaaagagt | |
| | ccgcacagggcggcataggcgaggcgatcgtcgacattcctgagattc | |
| | ctgggttcaaggacttggagcccatggagcagttcatcgcacaggtcg | |
| | atctgtgtgtggactgcacaactggctgcctcaaagggcttgccaacg | |
| | tgcagtgttctgacctgctcaagaagtggctgccgcaacgctgtgcga | |
| | cctttgccagcaagatccagggccaggtggacaagatcaagggggccg | |
| | gtggtgactaatcgatccctaccggttagtaatgagtttaaacggggg | |
| | aggctaactgaaacacggaaggagacaataccggaaggaacccgcgct | |
| | atgacggcaataaaaagacagaataaaacgcacgggtgttgggtcgtt | |
| | tgttcataaacgcggggttcggtcccagggctggcactctgtcgatac | |
| | cccaccgagaccccattggggccaatacgcccgcgtttcttccttttc | |
| | cccaccccaccccccaagttcgggtgaaggcccagggctcgcagccaa | |
| | cgtcggggcggcaggccctgccatagctttttggggcggcttttctcg | |
| | agtccttccttaaggacgtgctcgtcaatttttgttttgagagtcatc | |
| | tattcggatgcttttcatgaagttttttatgaccctgaccggcaccct | |
| | gcgcaaggccttcgccaccaccctggccgccgccatgctgatcggcac | |
| | cctggccggctgctcctccgccgcatacaacaagtctgacctcgtttc | |
| | gaagatcgcccagaagtccaacctgaccaaggctcaggccgaggctgc | |
| | tgttaacgccttccaggatgtgttcgtcgaggctatgaagtccggcga | |
| | aggcctgaagctcaccggcctgttctccgctgagcgcgtcaagcgccc | |
| | ggctcgcaccggccgcaacccgcgcactggcgagcagattgacattcc | |
| | ggcttcctacggcgttcgtatctccgctggctccctgctgaagaaggc | |
| | cgtcaccgagtatggacggaagaagcgcaggcagcgacggcgatgagg | |
| | gtttgcgcttgcgtcgtggagggagcggaacgccgaaaaaggatccac | |
| | gcgctggagatgttcaacgagtagatcgccacggcgacctccttccac | |
| | gcgtgcgggcacggggattctcaaggggccggcccgaggccccttgag | |
| | cccgccgggaggcgcccccggcagggcgggaatccaaagggcggagcc | |
| | ctgtggccctccccgggcaggggcgggatcgtcaagggcggagccctt | |
| | ggccccctcgggagagcgcactgacacaatgctacctccggtagcatt | |
| | aagtgcgccctccgccatgcggagggacgggccgcgaccggatatgcg | |
| | gggaacgtccacgacgcgtcttccgtgtcctccgtcctgccttgtgcc | |
| | gtcgattataatcctcggataacggacgatgattgaaccgattggagg | |
| | aaacgagatgccgaagagtttcgcgcagcagatcgaggacgacgagaa | |
| | caagatcaagcgcatccgggagcaccagcgcatggtcagggccaaaca | |
| | agccaaacaggagcgcaacgcccgcaccaaaaggctcgtggagaccgg | |
| | agcgatagtggaaaaagcgcacggcggcgcgtacgacgacgaaggacg | |
| | gcagacgttctcggatggtctgaacggcatcatctcggtctacgaccc | |
| | gtcccgcggcggcaacgtggacatgagagtcatcgacgtaatcgaccg | |
| | gcgcatccccagattgccaaggtccgaaaccacaacaggcacggcagc | |
| | ggcggcatcgcgaaccgtgcaagccaccgcaccacagccagcccacgc | |
| | ccaaccgcaaagcttcacgcccaacccccagcgcatcgaacaccggac | |
| | aggagcacagcagccggatcggtgggcctagccgggacggcgcagccc | |
| | tcgctccgctgggctgaaatatctgacttggttcgtaacatatttccg | |
| | acatgtcggaaatatgtcgtatcatcgttgctatgagtactgaacttc | |
| | gtgagcagtgggagcagctttacctgccgctgcgcccgctgtgcacga | |
| | acgacttcatcgagggcgtgtaccggcagcccagggcgaaggcgctgg | |
| | agggctaccggtacatcgaggcgaaccccaagaccgtcagcgacctgc | |
| | tcgtggtcgacatcgacgacgcgaacgcccgcgcgatggccctatggg | |
| | agcatgaggggatgctgcccaacgtgatcgtggagaacccgagaaacg | |
| | gccacgcgcacgccgtgtgggcgctggccgccccgttcagtcgcaccg | |
| | aatacgcgcggcgcaagccgctcgccctggccgcggcggtcacggagg | |
| | ggctgcgccgctcgtgcgacggggacaagggatattcggggctgatga | |
| | ccaagaacccgctgcacgaggcgtggaacagcgaactggtcaccgatc | |
| | acctgtacgggctggacgaactgcgcgaggcgctggaggagtccgggg | |
| | acatgccgccggcctcgtggaagcgcacgaaacggcgcaacgtggtcg | |
| | gattgggccgcaactgccatctgttcgagacggcgcgcacatgggcgt | |
| | acagggaggtgcgccaccactggggagacagcgaggggctgcggctgg | |
| | ccatctcggccgaggcgcacgaaatgaacgccgaactgttccccgagc | |
| | cactggcatggtcggaggtcgagcagatcgccaagagcatccacaagt | |
| | ggatcgtcacccaaagccgcatgtggcgcgacggccccaccgtctacg | |
| | aggcgacattcatcaccgtccaaagcgcccgagggaaaaagtcaggca | |
| | aggccagacgagaaacattcgaactattcgcgagcgaggagatgacgt | |
| | aa | |
| 44 | atgtgagcaaaaggccagcaaaaggccagggaccgtaaaaaggccgcg | DNA sequence of |
| | ttgctggcgtttttccataggctccgcccccctgacgagcatcacaaa | pFRG3.5-CMV-SGene |
| | aatcgacgctcaagtcagaggtggcgaaacccgacaggactataaaga | (artificial) |
| | taccaggcgtttccccctggaagctccctcgtgcgctctcctgttccg | E. coli Origin of |
| | accctgccgcttaccggatacctgtccgcctttctcccttcgggaagc | Replication |
| | gtggcgctttctcatagctcacgctgtaggtatctcagttcggtgtag | 1-692 bp |
| | gtcgttcgctccaagctgggctgtgtgcacgaaccccccgttcagccc | Spectinomycin Resistance |
| | gaccgctgcgccttatccggtaactatcgtcttgagtccaacccggta | Cassette 699-1734 bp |
| | agacacgacttatcgccactggcagcagccactggtaacaggattagc | CMV Enhancer/Promoter |
| | agagcgaggtatgtaggcggtgctacagagttcttgaagtggtggcct | sequence 1741-2467 bp |
| | aactacggctacactagaagaacagtatttggtatctgcgctctgctg | [CMV Enhancer |
| | aagccagttaccttcggaaaaagagttggtagctcttgatccggcaaa | 1791-2165 bp] |
| | caaaccaccgctggtagcggtggtttttttgtttgcaagcagcagatt | [CMV Promoter |
| | ggtaacggcgcagtggcggttttcatggcttgttatgactgttttttt | 2166-2385 bp] |
| | acgcgcagaaaaaaaggatctcaagaagatcctttgatcttttctacg | [CMV 5′UTR |
| | ggtttttttggggcggctttgaattcttttttttggggcggctttttt | 2386-2457 bp] |
| | tttatgcgctcacgcaactggtccagaaccttgaccgaacgcagcggt | S Gene (Spike) |
| | ggtaaatggcccgcctggcattatgcccagtacatgaccttatgggac | {SARS-CoV-2} 2478- |
| | ggggtacagtctatgcctcgggcatccaagcagcaagcgcgttacgcc | 6299 bp |
| | gtgggtcgatgtttgatgttatggagcagcaacgatgttacgcagcag | HSV-TK PolyA Signal |
| | ggcagtcgccctaaaacaaagttaaacatcatgagggaagcggtgatc | 6300-6620 bp |
| | gccgaagtatcgactcaactatcagaggtagttggcgtcatcgagcgc | HTP 6701-7102 bp |
| | catctcgaaccgacgttgctggccgtacatttgtacggctccgcagtg | [Secretion Signal |
| | gatggcggcctgaagccacacagtgatattgatttgctggttacggtg | 6701-6790 bp] |
| | accgtaaggcttgatgaaacaacgcggcgagctttgatcaacgacctt | [DNA Binding Domain |
| | ttggaaacttcggcttcccctggagagagcgagattctccgcgctgta | 6791-7066 bp] |
| | gaagtcaccattgttgtgcacgacgacatcattccgtggcgttatcca | [CPP 7067-7102 bp] |
| | gctaagcgcgaactgcaatttggagaatggcagcgcaatgacattctt | SynS Terminator |
| | gcaggtatcttcgagccagccacgatcgacattgatctggctatcttg | 7103-7144 bp |
| | ctgacaaaagcaagagaacatagcgttgccttggtaggtccagcggcg | Bif Origin of Replication |
| | gaggaactctttgatccggttcctgaacaggatctatttgaggcgcta | 7151-8978 bp |
| | aatgaaaccttaacgctatggaactcgccgcccgactgggctggcgat | |
| | gagcgaaatgtagtgcttacgttgtcccgcatttggtacagcgcagta | |
| | accggcaaaatcgcgccgaaggatgtcgctgccgactgggcaatggag | |
| | cgcctgccggcccagtatcagcccgtcatacttgaagctagacaggct | |
| | tatcttggacaagaagaagatcgcttggcctcgcgcgcagatcagttg | |
| | gaagaatttgtccactacgtgaaaggcgagatcaccaaggtagtcggc | |
| | aaataaggtaccgttaggcgttttcgcgatgtacgggccagatatacg | |
| | cgttgacattgattattgactagttattaatagtaatcaattacgggg | |
| | tcattagttcatagcccatatatggagttccgcgttacataacttacg | |
| | gtaaatggcccgcctggctgaccgcccaacgacccccgcccattgacg | |
| | tcaataatgacgtatgttcccatagtaacgccaatagggactttccat | |
| | tgacgtcaatgggtggagtatttacggtaaactgcccacttggcagta | |
| | catcaagtgtatcatatgccaagtacgccccctattgacgtcaatgac | |
| | ggtaaatggcccgcctggcattatgcccagtacatgaccttatgggac | |
| | tttcctacttggcagtacatctacgtattagtcatcgctattaccatg | |
| | gtgatgcggttttggcagtacatcaatgggcgtggatagcggtttgac | |
| | tcacggggatttccaagtctccaccccattgacgtcaatgggagtttg | |
| | ttttggcaccaaaatcaacgggactttccaaaatgtcgtaacaactcc | |
| | gccccattgacgcaaatgggcggtaggcgtgtacggtgggaggtctat | |
| | ataagcagagctcgtttagtgaaccgtcagatcgcctggagacgccat | |
| | ccacgctgttttgacctccatagaagacaccgggaccgatccagcctc | |
| | cggactctagaggatcgaagctagccaccatgtttgtttttcttgttt | |
| | tattgccactagtctctagtcagtgtgttaatcttacaaccagaactc | |
| | aattaccccctgcatacactaattctttcacacgtggtgtttattacc | |
| | ctgacaaagttttcagatcctcagttttacattcaactcaggacttgt | |
| | tcttacctttcttttccaatgttacttggttccatgctatacatgtct | |
| | ctgggaccaatggtactaagaggtttgataaccctgtcctaccattta | |
| | atgatggtgtttattttgcttccactgagaagtctaacataataagag | |
| | gctggatttttggtactactttagattcgaagacccagtccctactta | |
| | ttgttaataacgctactaatgttgttattaaagtctgtgaatttcaat | |
| | tttgtaatgatccatttttgggtgtttattaccacaaaaacaacaaaa | |
| | gttggatggaaagtgagttcagagtttattctagtgcgaataattgca | |
| | cttttgaatatgtctctcagccttttcttatggaccttgaaggaaaac | |
| | agggtaatttcaaaaatcttagggaatttgtgtttaagaatattgatg | |
| | gttattttaaaatatattctaagcacacgcctattaatttagtgcgtg | |
| | atctccctcagggtttttcggctttagaaccattggtagatttgccaa | |
| | taggtattaacatcactaggtttcaaactttacttgctttacatagaa | |
| | gttatttgactcctggtgattcttcttcaggttggacagctggtgctg | |
| | cagcttattatgtgggttatcttcaacctaggacttttctattaaaat | |
| | ataatgaaaatggaaccattacagatgctgtagactgtgcacttgacc | |
| | ctctctcagaaacaaagtgtacgttgaaatccttcactgtagaaaaag | |
| | gaatctatcaaacttctaactttagagtccaaccaacagaatctattg | |
| | ttagatttcctaatattacaaacttgtgcccttttggtgaagttttta | |
| | acgccaccagatttgcatctgtttatgcttggaacaggaagagaatca | |
| | gcaactgtgttgctgattattctgtcctatataattccgcatcatttt | |
| | ccacttttaagtgttatggagtgtctcctactaaattaaatgatctct | |
| | gctttactaatgtctatgcagattcatttgtaattagaggtgatgaag | |
| | tcagacaaatcgctccagggcaaactggaaagattgctgattataatt | |
| | ataaattaccagatgattttacaggctgcgttatagcttggaattcta | |
| | acaatcttgattctaaggttggtggtaattataattacctgtatagat | |
| | tgtttaggaagtctaatctcaaaccttttgagagagatatttcaactg | |
| | aaatctatcaggccggtagcacaccttgtaatggtgttgaaggtttta | |
| | attgttactttcctttacaatcatatggtttccaacccactaatggtg | |
| | ttggttaccaaccatacagagtagtagtactttcttttgaacttctac | |
| | atgcaccagcaactgtttgtggacctaaaaagtctactaatttggtta | |
| | aaaacaaatgtgtcaatttcaacttcaatggtttaacaggcacaggtg | |
| | ttcttactgagtctaacaaaaagtttctgcctttccaacaatttggca | |
| | gagacattgctgacactactgatgctgtccgtgatccacagacacttg | |
| | agattcttgacattacaccatgttcttttggtggtgtcagtgttataa | |
| | caccaggaacaaatacttctaaccaggttgctgttctttatcaggatg | |
| | ttaactgcacagaagtccctgttgctattcatgcagatcaacttactc | |
| | ctacttggcgtgtttattctacaggttctaatgtttttcaaacacgtg | |
| | caggctgtttaataggggctgaacatgtcaacaactcatatgagtgtg | |
| | acatacccattggtgcaggtatatgcgctagttatcagactcagacta | |
| | attctcctcggcgggcacgtagtgtagctagtcaatccatcattgcct | |
| | acactatgtcacttggtgcagaaaattcagttgcttactctaataact | |
| | ctattgccatacccacaaattttactattagtgttaccacagaaattc | |
| | taccagtgtctatgaccaagacatcagtagattgtacaatgtacattt | |
| | gtggtgattcaactgaatgcagcaatcttttgttgcaatatggcagtt | |
| | tttgtacacaattaaaccgtgctttaactggaatagctgttgaacaag | |
| | acaaaaacacccaagaagtttttgcacaagtcaaacaaatttacaaaa | |
| | caccaccaattaaagattttggtggttttaatttttcacaaatattac | |
| | cagatccatcaaaaccaagcaagaggtcatttattgaagatctacttt | |
| | tcaacaaagtgacacttgcagatgctggcttcatcaaacaatatggtg | |
| | attgccttggtgatattgctgctagagacctcatttgtgcacaaaagt | |
| | ttaacggccttactgttttgccacctttgctcacagatgaaatgattg | |
| | ctcaatacacttctgcactgttagcgggtacaatcacttctggttgga | |
| | cctttggtgcaggtgctgcattacaaataccatttgctatgcaaatgg | |
| | cttataggtttaatggtattggagttacacagaatgttctctatgaga | |
| | accaaaaattgattgccaaccaatttaatagtgctattggcaaaattc | |
| | aagactcactttcttccacagcaagtgcacttggaaaacttcaagatg | |
| | tggtcaaccaaaatgcacaagctttaaacacgcttgttaaacaactta | |
| | gctccaattttggtgcaatttcaagtgttttaaatgatatcctttcac | |
| | gtcttgacaaagttgaggctgaagtgcaaattgataggttgatcacag | |
| | gcagacttcaaagtttgcagacatatgtgactcaacaattaattagag | |
| | ctgcagaaatcagagcttctgctaatcttgctgctactaaaatgtcag | |
| | agtgtgtacttggacaatcaaaaagagttgatttttgtggaaagggct | |
| | atcatcttatgtccttccctcagtcagcacctcatggtgtagtcttct | |
| | tgcatgtgacttatgtccctgcacaagaaaagaacttcacaactgctc | |
| | ctgccatttgtcatgatggaaaagcacactttcctcgtgaaggtgtct | |
| | ttgtttcaaatggcacacactggtttgtaacacaaaggaatttttatg | |
| | aaccacaaatcattactacagacaacacatttgtgtctggtaactgtg | |
| | atgttgtaataggaattgtcaacaacacagtttatgatcctttgcaac | |
| | ctgaattagactcattcaaggaggagttagataaatattttaagaatc | |
| | atacatcaccagatgttgatttaggtgacatctctggcattaatgctt | |
| | cagttgtaaacattcaaaaagaaattgaccgcctcaatgaggttgcca | |
| | agaatttaaatgaatctctcatcgatctccaagaacttggaaagtatg | |
| | agcagtatataaaatggccatggtacatttggctaggttttatagctg | |
| | gcttgattgccatagtaatggtgacaattatgctttgctgtatgacca | |
| | gttgctgtagttgtctcaagggctgttgttcttgtggatcctgctgca | |
| | aatttgatgaagacgactctgagccagtgctcaaaggagtcaaattac | |
| | attacacataatcgatccctaccggttagtaatgagtttaaacggggg | |
| | aggctaactgaaacacggaaggagacaataccggaaggaacccgcgct | |
| | atgacggcaataaaaagacagaataaaacgcacgggtgttgggtcgtt | |
| | tgttcataaacgcggggttcggtcccagggctggcactctgtcgatac | |
| | cccaccgagaccccattggggccaatacgcccgcgtttcttccttttc | |
| | cccaccccaccccccaagttcgggtgaaggcccagggctcgcagccaa | |
| | cgtcggggcggcaggccctgccatagctttttggggcggcttttctcg | |
| | agtccttccttaaggacgtgctcgtcaatttttgttttgagagtcatc | |
| | tattcggatgcttttcatgaagttttttatgaccctgaccggcaccct | |
| | gcgcaaggccttcgccaccaccctggccgccgccatgctgatcggcac | |
| | cctggccggctgctcctccgccgcatacaacaagtctgacctcgtttc | |
| | gaagatcgcccagaagtccaacctgaccaaggctcaggccgaggctgc | |
| | tgttaacgccttccaggatgtgttcgtcgaggctatgaagtccggcga | |
| | aggcctgaagctcaccggcctgttctccgctgagcgcgtcaagcgccc | |
| | ggctcgcaccggccgcaacccgcgcactggcgagcagattgacattcc | |
| | ggcttcctacggcgttcgtatctccgctggctccctgctgaagaaggc | |
| | cgtcaccgagtatggacggaagaagcgcaggcagcgacggcgatgagg | |
| | gtttgcgcttgcgtcgtggagggagcggaacgccgaaaaaggatccac | |
| | gcgctggagatgttcaacgagtagatcgccacggcgacctccttccac | |
| | gcgtgcgggcacggggattctcaaggggccggcccgaggccccttgag | |
| | cccgccgggaggcgcccccggcagggcgggaatccaaagggcggagcc | |
| | ctgtggccctccccgggcaggggcgggatcgtcaagggcggagccctt | |
| | ggccccctcgggagagcgcactgacacaatgctacctccggtagcatt | |
| | aagtgcgccctccgccatgcggagggacgggccgcgaccggatatgcg | |
| | gggaacgtccacgacgcgtcttccgtgtcctccgtcctgccttgtgcc | |
| | gtcgattataatcctcggataacggacgatgattgaaccgattggagg | |
| | aaacgagatgccgaagagtttcgcgcagcagatcgaggacgacgagaa | |
| | caagatcaagcgcatccgggagcaccagcgcatggtcagggccaaaca | |
| | agccaaacaggagcgcaacgcccgcaccaaaaggctcgtggagaccgg | |
| | agcgatagtggaaaaagcgcacggcggcgcgtacgacgacgaaggacg | |
| | gcagacgttctcggatggtctgaacggcatcatctcggtctacgaccc | |
| | gtcccgcggcggcaacgtggacatgagagtcatcgacgtaatcgaccg | |
| | gcgcatccccagattgccaaggtccgaaaccacaacaggcacggcagc | |
| | ggcggcatcgcgaaccgtgcaagccaccgcaccacagccagcccacgc | |
| | ccaaccgcaaagcttcacgcccaacccccagcgcatcgaacaccggac | |
| | aggagcacagcagccggatcggtgggcctagccgggacggcgcagccc | |
| | tcgctccgctgggctgaaatatctgacttggttcgtaacatatttccg | |
| | acatgtcggaaatatgtcgtatcatcgttgctatgagtactgaacttc | |
| | gtgagcagtgggagcagctttacctgccgctgcgcccgctgtgcacga | |
| | acgacttcatcgagggcgtgtaccggcagcccagggcgaaggcgctgg | |
| | agggctaccggtacatcgaggcgaaccccaagaccgtcagcgacctgc | |
| | tcgtggtcgacatcgacgacgcgaacgcccgcgcgatggccctatggg | |
| | agcatgaggggatgctgcccaacgtgatcgtggagaacccgagaaacg | |
| | gccacgcgcacgccgtgtgggcgctggccgccccgttcagtcgcaccg | |
| | aatacgcgcggcgcaagccgctcgccctggccgcggcggtcacggagg | |
| | ggctgcgccgctcgtgcgacggggacaagggatattcggggctgatga | |
| | ccaagaacccgctgcacgaggcgtggaacagcgaactggtcaccgatc | |
| | acctgtacgggctggacgaactgcgcgaggcgctggaggagtccgggg | |
| | acatgccgccggcctcgtggaagcgcacgaaacggcgcaacgtggtcg | |
| | gattgggccgcaactgccatctgttcgagacggcgcgcacatgggcgt | |
| | acagggaggtgcgccaccactggggagacagcgaggggctgcggctgg | |
| | ccatctcggccgaggcgcacgaaatgaacgccgaactgttccccgagc | |
| | cactggcatggtcggaggtcgagcagatcgccaagagcatccacaagt | |
| | ggatcgtcacccaaagccgcatgtggcgcgacggccccaccgtctacg | |
| | aggcgacattcatcaccgtccaaagcgcccgagggaaaaagtcaggca | |
| | aggccagacgagaaacattcgaactattcgcgagcgaggagatgacgt | |
| | aa | |
| 45 | atgtgagcaaaaggccagcaaaaggccagggaccgtaaaaaggccgcg | DNA sequence of |
| | ttgctggcgtttttccataggctccgcccccctgacgagcatcacaaa | pFRG3.5-CMV- |
| | aatcgacgctcaagtcagaggtggcgaaacccgacaggactataaaga | COVID19-SIMIN |
| | taccaggcgtttccccctggaagctccctcgtgcgctctcctgttccg | (artificial) |
| | accctgccgcttaccggatacctgtccgcctttctcccttcgggaagc | E. coli Origin of |
| | gtggcgctttctcatagctcacgctgtaggtatctcagttcggtgtag | Replication |
| | gtcgttcgctccaagctgggctgtgtgcacgaaccccccgttcagccc | 1-692 bp |
| | gaccgctgcgccttatccggtaactatcgtcttgagtccaacccggta | Spectinomycin Resistance |
| | agacacgacttatcgccactggcagcagccactggtaacaggattagc | Cassette 699-1734 bp |
| | agagcgaggtatgtaggcggtgctacagagttcttgaagtggtggcct | CMV Enhancer/Promoter |
| | aactacggctacactagaagaacagtatttggtatctgcgctctgctg | sequence 1741-2467 bp |
| | aagccagttaccttcggaaaaagagttggtagctcttgatccggcaaa | [CMV Enhancer |
| | caaaccaccgctggtagcggtggtttttttgtttgcaagcagcagatt | 1791-2165 bp] |
| | acgcgcagaaaaaaaggatctcaagaagatcctttgatcttttctacg | [CMV Promoter |
| | ggtttttttggggcggctttgaattcttttttttggggcggctttttt | 2166-2385 bp] |
| | tttatgcgctcacgcaactggtccagaaccttgaccgaacgcagcggt | [CMV 5′UTR |
| | ggtaacggcgcagtggcggttttcatggcttgttatgactgttttttt | 2386-2457 bp] |
| | ggggtacagtctatgcctcgggcatccaagcagcaagcgcgttacgcc | SiMiN poly-cistronic Gene |
| | gtgggtcgatgtttgatgttatggagcagcaacgatgttacgcagcag | {SARS-CoV-2} |
| | ggcagtcgccctaaaacaaagttaaacatcatgagggaagcggtgatc | 2478-9386 bp |
| | gccgaagtatcgactcaactatcagaggtagttggcgtcatcgagcgc | [S Gene (Spike) |
| | catctcgaaccgacgttgctggccgtacatttgtacggctccgcagtg | 2478-6299 bp] |
| | gatggcggcctgaagccacacagtgatattgatttgctggttacggtg | [IRES 6300-6878 bp] |
| | accgtaaggcttgatgaaacaacgcggcgagctttgatcaacgacctt | [M Gene (Matrix) |
| | ttggaaacttcggcttcccctggagagagcgagattctccgcgctgta | 6879-7547 bp] |
| | gaagtcaccattgttgtgcacgacgacatcattccgtggcgttatcca | [IRES 7548-8126 bp] |
| | gctaagcgcgaactgcaatttggagaatggcagcgcaatgacattctt | [N Gene (Nucleocapsid) |
| | gcaggtatcttcgagccagccacgatcgacattgatctggctatcttg | 8127-9386 bp] |
| | ctgacaaaagcaagagaacatagcgttgccttggtaggtccagcggcg | HSV-TK PolyA Signal |
| | gaggaactctttgatccggttcctgaacaggatctatttgaggcgcta | 9387-9707 bp |
| | aatgaaaccttaacgctatggaactcgccgcccgactgggctggcgat | Bacterial Rb Promoter |
| | gagcgaaatgtagtgcttacgttgtcccgcatttggtacagcgcagta | 9714-9787 bp |
| | accggcaaaatcgcgccgaaggatgtcgctgccgactgggcaatggag | HTP 9788-10189 bp |
| | cgcctgccggcccagtatcagcccgtcatacttgaagctagacaggct | [Secretion Signal |
| | tatcttggacaagaagaagatcgcttggcctcgcgcgcagatcagttg | 9788-9877 bp] |
| | gaagaatttgtccactacgtgaaaggcgagatcaccaaggtagtcggc | [DNA Binding Domain |
| | aaataaggtaccgttaggcgttttcgcgatgtacgggccagatatacg | 9878-10153 bp] |
| | cgttgacattgattattgactagttattaatagtaatcaattacgggg | [CPP 10154-10189 bp] |
| | tcattagttcatagcccatatatggagttccgcgttacataacttacg | SynS Terminator |
| | gtaaatggcccgcctggctgaccgcccaacgacccccgcccattgacg | 10190-10231 bp |
| | tcaataatgacgtatgttcccatagtaacgccaatagggactttccat | Bif Origin of Replication |
| | tgacgtcaatgggtggagtatttacggtaaactgcccacttggcagta | 10238-12065 bp |
| | catcaagtgtatcatatgccaagtacgccccctattgacgtcaatgac | |
| | ggtaaatggcccgcctggcattatgcccagtacatgaccttatgggac | |
| | tttcctacttggcagtacatctacgtattagtcatcgctattaccatg | |
| | gtgatgcggttttggcagtacatcaatgggcgtggatagcggtttgac | |
| | tcacggggatttccaagtctccaccccattgacgtcaatgggagtttg | |
| | ttttggcaccaaaatcaacgggactttccaaaatgtcgtaacaactcc | |
| | gccccattgacgcaaatgggcggtaggcgtgtacggtgggaggtctat | |
| | ataagcagagctcgtttagtgaaccgtcagatcgcctggagacgccat | |
| | ccacgctgttttgacctccatagaagacaccgggaccgatccagcctc | |
| | cggactctagaggatcgaagctagccaccatgtttgtttttcttgttt | |
| | tattgccactagtctctagtcagtgtgttaatcttacaaccagaactc | |
| | aattaccccctgcatacactaattctttcacacgtggtgtttattacc | |
| | ctgacaaagttttcagatcctcagttttacattcaactcaggacttgt | |
| | tcttacctttcttttccaatgttacttggttccatgctatacatgtct | |
| | ctgggaccaatggtactaagaggtttgataaccctgtcctaccattta | |
| | atgatggtgtttattttgcttccactgagaagtctaacataataagag | |
| | gctggatttttggtactactttagattcgaagacccagtccctactta | |
| | ttgttaataacgctactaatgttgttattaaagtctgtgaatttcaat | |
| | tttgtaatgatccatttttgggtgtttattaccacaaaaacaacaaaa | |
| | gttggatggaaagtgagttcagagtttattctagtgcgaataattgca | |
| | cttttgaatatgtctctcagccttttcttatggaccttgaaggaaaac | |
| | agggtaatttcaaaaatcttagggaatttgtgtttaagaatattgatg | |
| | gttattttaaaatatattctaagcacacgcctattaatttagtgcgtg | |
| | atctccctcagggtttttcggctttagaaccattggtagatttgccaa | |
| | taggtattaacatcactaggtttcaaactttacttgctttacatagaa | |
| | gttatttgactcctggtgattcttcttcaggttggacagctggtgctg | |
| | cagcttattatgtgggttatcttcaacctaggacttttctattaaaat | |
| | ataatgaaaatggaaccattacagatgctgtagactgtgcacttgacc | |
| | ctctctcagaaacaaagtgtacgttgaaatccttcactgtagaaaaag | |
| | gaatctatcaaacttctaactttagagtccaaccaacagaatctattg | |
| | ttagatttcctaatattacaaacttgtgcccttttggtgaagttttta | |
| | acgccaccagatttgcatctgtttatgcttggaacaggaagagaatca | |
| | gcaactgtgttgctgattattctgtcctatataattccgcatcatttt | |
| | ccacttttaagtgttatggagtgtctcctactaaattaaatgatctct | |
| | gctttactaatgtctatgcagattcatttgtaattagaggtgatgaag | |
| | tcagacaaatcgctccagggcaaactggaaagattgctgattataatt | |
| | ataaattaccagatgattttacaggctgcgttatagcttggaattcta | |
| | acaatcttgattctaaggttggtggtaattataattacctgtatagat | |
| | tgtttaggaagtctaatctcaaaccttttgagagagatatttcaactg | |
| | aaatctatcaggccggtagcacaccttgtaatggtgttgaaggtttta | |
| | attgttactttcctttacaatcatatggtttccaacccactaatggtg | |
| | ttggttaccaaccatacagagtagtagtactttcttttgaacttctac | |
| | atgcaccagcaactgtttgtggacctaaaaagtctactaatttggtta | |
| | aaaacaaatgtgtcaatttcaacttcaatggtttaacaggcacaggtg | |
| | ttcttactgagtctaacaaaaagtttctgcctttccaacaatttggca | |
| | gagacattgctgacactactgatgctgtccgtgatccacagacacttg | |
| | agattcttgacattacaccatgttcttttggtggtgtcagtgttataa | |
| | caccaggaacaaatacttctaaccaggttgctgttctttatcaggatg | |
| | ttaactgcacagaagtccctgttgctattcatgcagatcaacttactc | |
| | ctacttggcgtgtttattctacaggttctaatgtttttcaaacacgtg | |
| | caggctgtttaataggggctgaacatgtcaacaactcatatgagtgtg | |
| | acatacccattggtgcaggtatatgcgctagttatcagactcagacta | |
| | attctcctcggcgggcacgtagtgtagctagtcaatccatcattgcct | |
| | acactatgtcacttggtgcagaaaattcagttgcttactctaataact | |
| | ctattgccatacccacaaattttactattagtgttaccacagaaattc | |
| | taccagtgtctatgaccaagacatcagtagattgtacaatgtacattt | |
| | gtggtgattcaactgaatgcagcaatcttttgttgcaatatggcagtt | |
| | tttgtacacaattaaaccgtgctttaactggaatagctgttgaacaag | |
| | acaaaaacacccaagaagtttttgcacaagtcaaacaaatttacaaaa | |
| | caccaccaattaaagattttggtggttttaatttttcacaaatattac | |
| | cagatccatcaaaaccaagcaagaggtcatttattgaagatctacttt | |
| | tcaacaaagtgacacttgcagatgctggcttcatcaaacaatatggtg | |
| | attgccttggtgatattgctgctagagacctcatttgtgcacaaaagt | |
| | ttaacggccttactgttttgccacctttgctcacagatgaaatgattg | |
| | ctcaatacacttctgcactgttagcgggtacaatcacttctggttgga | |
| | cctttggtgcaggtgctgcattacaaataccatttgctatgcaaatgg | |
| | cttataggtttaatggtattggagttacacagaatgttctctatgaga | |
| | accaaaaattgattgccaaccaatttaatagtgctattggcaaaattc | |
| | aagactcactttcttccacagcaagtgcacttggaaaacttcaagatg | |
| | tggtcaaccaaaatgcacaagctttaaacacgcttgttaaacaactta | |
| | gctccaattttggtgcaatttcaagtgttttaaatgatatcctttcac | |
| | gtcttgacaaagttgaggctgaagtgcaaattgataggttgatcacag | |
| | gcagacttcaaagtttgcagacatatgtgactcaacaattaattagag | |
| | ctgcagaaatcagagcttctgctaatcttgctgctactaaaatgtcag | |
| | agtgtgtacttggacaatcaaaaagagttgatttttgtggaaagggct | |
| | atcatcttatgtccttccctcagtcagcacctcatggtgtagtcttct | |
| | tgcatgtgacttatgtccctgcacaagaaaagaacttcacaactgctc | |
| | ctgccatttgtcatgatggaaaagcacactttcctcgtgaaggtgtct | |
| | ttgtttcaaatggcacacactggtttgtaacacaaaggaatttttatg | |
| | aaccacaaatcattactacagacaacacatttgtgtctggtaactgtg | |
| | atgttgtaataggaattgtcaacaacacagtttatgatcctttgcaac | |
| | ctgaattagactcattcaaggaggagttagataaatattttaagaatc | |
| | atacatcaccagatgttgatttaggtgacatctctggcattaatgctt | |
| | cagttgtaaacattcaaaaagaaattgaccgcctcaatgaggttgcca | |
| | agaatttaaatgaatctctcatcgatctccaagaacttggaaagtatg | |
| | agcagtatataaaatggccatggtacatttggctaggttttatagctg | |
| | gcttgattgccatagtaatggtgacaattatgctttgctgtatgacca | |
| | gttgctgtagttgtctcaagggctgttgttcttgtggatcctgctgca | |
| | aatttgatgaagacgactctgagccagtgctcaaaggagtcaaattac | |
| | attacacataacgccccccccccctaacgttactggccgaagccgctt | |
| | ggaataaggccggtgtgcgtttgtctatatgttattttccaccatatt | |
| | gccgtcttttggcaatgtgagggcccggaaacctggccctgtcttctt | |
| | gacgagcattcctaggggtctttcccctctcgccaaaggaatgcaagg | |
| | tctgttgaatgtcgtgaaggaagcagttcctctggaagcttcttgaag | |
| | acaaacaacgtctgtagcgaccctttgcaggcagcggaaccccccacc | |
| | tggcgacaggtgcctctgcggccaaaagccacgtgtataagatacacc | |
| | tgcaaaggcggcacaaccccagtgccacgttgtgagttggatagttgt | |
| | ggaaagagtcaaatggctctcctcaagcgtattcaacaaggggctgaa | |
| | ggatgcccagaaggtaccccattgtatgggatctgatctggggcctcg | |
| | gtgcacatgctttacatgtgtttagtcgaggttaaaaaaacgtctagg | |
| | ccccccgaaccacggggacgtggttttcctttgaaaaacacgatgata | |
| | atatggccacaaccatggcagattccaacggtactattaccgttgaag | |
| | agcttaaaaagctccttgaacaatggaacctagtaataggtttcctat | |
| | tccttacatggatttgtcttctacaatttgcctatgccaacaggaata | |
| | ggtttttgtatataattaagttaattttcctctggctgttatggccag | |
| | taactttagcttgttttgtgcttgctgctgtttacagaataaattgga | |
| | tcaccggtggaattgctatcgcaatggcttgtcttgtaggcttgatgt | |
| | ggctcagctacttcattgcttctttcagactgtttgcgcgtacgcgtt | |
| | ccatgtggtcattcaatccagaaactaacattcttctcaacgtgccac | |
| | tccatggcactattctgaccagaccgcttctagaaagtgaactcgtaa | |
| | tcggagctgtgatccttcgtggacatcttcgtattgctggacaccatc | |
| | taggacgctgtgacatcaaggacctgcctaaagaaatcactgttgcta | |
| | catcacgaacgctttcttattacaaattgggagcttcgcagcgtgtag | |
| | caggtgactcaggttttgctgcatacagtcgctacaggattggcaact | |
| | ataaattaaacacagaccattccagtagcagtgacaatattgctttgc | |
| | ttgtacagtaacgccccccccccctaacgttactggccgaagccgctt | |
| | ggaataaggccggtgtgcgtttgtctatatgttattttccaccatatt | |
| | gccgtcttttggcaatgtgagggcccggaaacctggccctgtcttctt | |
| | gacgagcattcctaggggtctttcccctctcgccaaaggaatgcaagg | |
| | tctgttgaatgtcgtgaaggaagcagttcctctggaagcttcttgaag | |
| | acaaacaacgtctgtagcgaccctttgcaggcagcggaaccccccacc | |
| | tggcgacaggtgcctctgcggccaaaagccacgtgtataagatacacc | |
| | tgcaaaggcggcacaaccccagtgccacgttgtgagttggatagttgt | |
| | ggaaagagtcaaatggctctcctcaagcgtattcaacaaggggctgaa | |
| | ggatgcccagaaggtaccccattgtatgggatctgatctggggcctcg | |
| | gtgcacatgctttacatgtgtttagtcgaggttaaaaaaacgtctagg | |
| | ccccccgaaccacggggacgtggttttcctttgaaaaacacgatgata | |
| | atatggccacaaccatgtctgataatggaccccaaaatcagcgaaatg | |
| | caccccgcattacgtttggtggaccctcagattcaactggcagtaacc | |
| | agaatggagaacgcagtggggcgcgatcaaaacaacgtcggccccaag | |
| | gtttacccaataatactgcgtcttggttcaccgctctcactcaacatg | |
| | gcaaggaagaccttaaattccctcgaggacaaggcgttccaattaaca | |
| | ccaatagcagtccagatgaccaaattggctactaccgaagagctacca | |
| | gacgaattcgtggtggtgacggtaaaatgaaagatctcagtccaagat | |
| | ggtatttctactacctaggaactgggccagaagctggacttccctatg | |
| | gtgctaacaaagacggcatcatatgggttgcaactgagggagccttga | |
| | atacaccaaaagatcacattggcacccgcaatcctgctaacaatgctg | |
| | caatcgtgctacaacttcctcaaggaacaacattgccaaaaggcttct | |
| | acgcagaagggagcagaggcggcagtcaagcctcttctcgttcctcat | |
| | cacgtagtcgcaacagttcaagaaattcaactccaggcagcagtaggg | |
| | gaacttctcctgctagaatggctggcaatggcggtgatgctgctcttg | |
| | ctttgctgctgcttgacagattgaaccagcttgagagcaaaatgtctg | |
| | gtaaaggccaacaacaacaaggccaaactgtcactaagaaatctgctg | |
| | ctgaggcttctaagaagcctcggcaaaaacgtactgccactaaagcat | |
| | acaatgtaacacaagctttcggcagacgtggtccagaacaaacccaag | |
| | gaaattttggggaccaggaactaatcagacaaggaactgattacaaac | |
| | attggccgcaaattgcacaatttgcccccagcgcttcagcgttcttcg | |
| | gaatgtcgcgcattggcatggaagtcacaccttcgggaacgtggttga | |
| | cctacacaggtgccatcaaattggatgacaaagatccaaatttcaaag | |
| | atcaagtcattttgctgaataagcatattgacgcatacaaaacattcc | |
| | caccaacagagcctaaaaaggacaaaaagaagaaggctgatgaaactc | |
| | aagccttaccgcagagacagaagaaacagcaaactgtgactcttcttc | |
| | ctgctgcagatttggatgatttctccaaacaattgcaacaatccatga | |
| | gcagtgctgactcaactcaggcctaatcgatccctaccggttagtaat | |
| | gagtttaaacgggggaggctaactgaaacacggaaggagacaataccg | |
| | gaaggaacccgcgctatgacggcaataaaaagacagaataaaacgcac | |
| | gggtgttgggtcgtttgttcataaacgcggggttcggtcccagggctg | |
| | gcactctgtcgataccccaccgagaccccattggggccaatacgcccg | |
| | cgtttcttccttttccccaccccaccccccaagttcgggtgaaggccc | |
| | agggctcgcagccaacgtcggggcggcaggccctgccatagctttttg | |
| | gggcggcttttctcgagtccttccttaaggacgtgctcgtcaattttt | |
| | gttttgagagtcatctattcggatgcttttcatgaagttttttatgac | |
| | cctgaccggcaccctgcgcaaggccttcgccaccaccctggccgccgc | |
| | catgctgatcggcaccctggccggctgctcctccgccgcatacaacaa | |
| | gtctgacctcgtttcgaagatcgcccagaagtccaacctgaccaaggc | |
| | tcaggccgaggctgctgttaacgccttccaggatgtgttcgtcgaggc | |
| | tatgaagtccggcgaaggcctgaagctcaccggcctgttctccgctga | |
| | gcgcgtcaagcgcccggctcgcaccggccgcaacccgcgcactggcga | |
| | gcagattgacattccggcttcctacggcgttcgtatctccgctggctc | |
| | cctgctgaagaaggccgtcaccgagtatggacggaagaagcgcaggca | |
| | gcgacggcgatgagggtttgcgcttgcgtcgtggagggagcggaacgc | |
| | cgaaaaaggatccacgcgctggagatgttcaacgagtagatcgccacg | |
| | gcgacctccttccacgcgtgcgggcacggggattctcaaggggccggc | |
| | ccgaggccccttgagcccgccgggaggcgcccccggcagggcgggaat | |
| | ccaaagggcggagccctgtggccctccccgggcaggggcgggatcgtc | |
| | aagggcggagcccttggccccctcgggagagcgcactgacacaatgct | |
| | acctccggtagcattaagtgcgccctccgccatgcggagggacgggcc | |
| | gcgaccggatatgcggggaacgtccacgacgcgtcttccgtgtcctcc | |
| | gtcctgccttgtgccgtcgattataatcctcggataacggacgatgat | |
| | tgaaccgattggaggaaacgagatgccgaagagtttcgcgcagcagat | |
| | cgaggacgacgagaacaagatcaagcgcatccgggagcaccagcgcat | |
| | ggtcagggccaaacaagccaaacaggagcgcaacgcccgcaccaaaag | |
| | gctcgtggagaccggagcgatagtggaaaaagcgcacggcggcgcgta | |
| | cgacgacgaaggacggcagacgttctcggatggtctgaacggcatcat | |
| | ctcggtctacgacccgtcccgcggcggcaacgtggacatgagagtcat | |
| | cgacgtaatcgaccggcgcatccccagattgccaaggtccgaaaccac | |
| | aacaggcacggcagcggcggcatcgcgaaccgtgcaagccaccgcacc | |
| | acagccagcccacgcccaaccgcaaagcttcacgcccaacccccagcg | |
| | catcgaacaccggacaggagcacagcagccggatcggtgggcctagcc | |
| | gggacggcgcagccctcgctccgctgggctgaaatatctgacttggtt | |
| | cgtaacatatttccgacatgtcggaaatatgtcgtatcatcgttgcta | |
| | tgagtactgaacttcgtgagcagtgggagcagctttacctgccgctgc | |
| | gcccgctgtgcacgaacgacttcatcgagggcgtgtaccggcagccca | |
| | gggcgaaggcgctggagggctaccggtacatcgaggcgaaccccaaga | |
| | ccgtcagcgacctgctcgtggtcgacatcgacgacgcgaacgcccgcg | |
| | cgatggccctatgggagcatgaggggatgctgcccaacgtgatcgtgg | |
| | agaacccgagaaacggccacgcgcacgccgtgtgggcgctggccgccc | |
| | cgttcagtcgcaccgaatacgcgcggcgcaagccgctcgccctggccg | |
| | cggcggtcacggaggggctgcgccgctcgtgcgacggggacaagggat | |
| | attcggggctgatgaccaagaacccgctgcacgaggcgtggaacagcg | |
| | aactggtcaccgatcacctgtacgggctggacgaactgcgcgaggcgc | |
| | tggaggagtccggggacatgccgccggcctcgtggaagcgcacgaaac | |
| | ggcgcaacgtggtcggattgggccgcaactgccatctgttcgagacgg | |
| | cgcgcacatgggcgtacagggaggtgcgccaccactggggagacagcg | |
| | aggggctgcggctggccatctcggccgaggcgcacgaaatgaacgccg | |
| | aactgttccccgagccactggcatggtcggaggtcgagcagatcgcca | |
| | agagcatccacaagtggatcgtcacccaaagccgcatgtggcgcgacg | |
| | gccccaccgtctacgaggcgacattcatcaccgtccaaagcgcccgag | |
| | ggaaaaagtcaggcaaggccagacgagaaacattcgaactattcgcga | |
| | gcgaggagatgacgtaa | |
| 46 | MFVFLVLLPLVSSQCVNLTTRTQLPPAYTNSFTRGVYYPDKVFRSSVL | SARS-CoV-2 (wildtype) |
| | HSTQDLFLPFFSNVTWFHAIHVSGTNGTKRFDNPVLPFNDGVYFASTE | spike protein |
| | KSNIIRGWIFGTTLDSKTQSLLIVNNATNVVIKVCEFQFCNDPFLGVY | Signal Peptide/Secretion |
| | YHKNNKSWMESEFRVYSSANNCTFEYVSQPFLMDLEGKQGNFKNLREF | Signal 1-12 aa |
| | VFKNIDGYFKIYSKHTPINLVRDLPQGFSALEPLVDLPIGINITRFQT | S1 domain 13-685 aa |
| | LLALHRSYLTPGDSSSGWTAGAAAYYVGYLQPRTFLLKYNENGTITDA | (cleavage site at R685- |
| | VDCALDPLSETKCTLKSFTVEKGIYQTSNFRVQPTESIVRFPNITNLC | S686) |
| | PFGEVFNATRFASVYAWNRKRISNCVADYSVLYNSASFSTFKCYGVSP | S2 domain 686-1273 aa |
| | TKLNDLCFTNVYADSFVIRGDEVRQIAPGQTGKIADYNYKLPDDFTGC | (cleavage site at R815- |
| | VIAWNSNNLDSKVGGNYNYLYRLFRKSNLKPFERDISTEIYQAGSTPC | S816) |
| | NGVEGFNCYFPLQSYGFQPTNGVGYQPYRVVVLSFELLHAPATVCGPK | S2′ domain 816-1273 aa |
| | KSTNLVKNKCVNFNFNGLTGTGVLTESNKKFLPFQQFGRDIADTTDAV | Receptor Binding Domain |
| | RDPQTLEILDITPCSFGGVSVITPGTNTSNQVAVLYQDVNCTEVPVAI | 319-541 aa |
| | HADQLTPTWRVYSTGSNVFQTRAGCLIGAEHVNNSYECDIPIGAGICA | Receptor Binding Motif |
| | SYQTQTNSPRRARSVASQSIIAYTMSLGAENSVAYSNNSIAIPTNFTI | 437-508 aa |
| | SVTTEILPVSMTKTSVDCTMYICGDSTECSNLLLQYGSFCTQLNRALT | Transmembrane Domain |
| | GIAVEQDKNTQEVFAQVKQIYKTPPIKDFGGFNFSQILPDPSKPSKRS | 1214-1234 aa |
| | FIEDLLFNKVTLADAGFIKQYGDCLGDIAARDLICAQKENGLTVLPPL | Cytoplasmic Domain |
| | LTDEMIAQYTSALLAGTITSGWTFGAGAALQIPFAMQMAYRFNGIGVT | 1245-1273 aa |
| | QNVLYENQKLIANQFNSAIGKIQDSLSSTASALGKLQDVVNQNAQALN | |
| | TLVKQLSSNFGAISSVLNDILSRLDKVEAEVQIDRLITGRLQSLQTYV | |
| | TQQLIRAAEIRASANLAATKMSECVLGQSKRVDFCGKGYHLMSFPQSA | |
| | PHGVVFLHVTYVPAQEKNFTTAPAICHDGKAHFPREGVFVSNGTHWFV | |
| | TQRNFYEPQIITTDNTFVSGNCDVVIGIVNNTVYDPLQPELDSFKEEL | |
| | DKYFKNHTSPDVDLGDISGINASVVNIQKEIDRLNEVAKNLNESLIDL | |
| | QELGKYEQYIKWPWYIWLGFIAGLIAIVMVTMLCCMTSCCSCLKGCC | |
| | SCGSCCKFDEDDSEPVLKGVKLHYT | |
| 47 | MADSNGTITVEELKKLLEQWNLVIGFLFLTWICLLQFAYANRNRFLYI | SARS-CoV-2 (wildtype) |
| | IKLIFLWLLWPVTLACFVLAAVYRINWITGGIAIAMACLVGLMWLSYF | matrix protein |
| | IASFRLFARTRSMWSFNPETNILLNVPLHGTILTRPLLESELVIGAVI | |
| | LRGHLRIAGHHLGRCDIKDLPKEITVATSRTLSYYKLGASQRVAGDSG | |
| | FAAYSRYRIGNYKLNTDHSSSSDNIALLVQ | |
| 48 | MSDNGPQNQRNAPRITFGGPSDSTGSNQNGERSGARSKQRRPQGLPNN | SARS-CoV-2 (wildtype) |
| | TASWFTALTQHGKEDLKFPRGQGVPINTNSSPDDQIGYYRRATRRIRG | nucleocapsid protein |
| | GDGKMKDLSPRWYFYYLGTGPEAGLPYGANKDGIIWVATEGALNTPKD | |
| | HIGTRNPANNAAIVLQLPQGTTLPKGFYAEGSRGGSQASSRSSSRSRN | |
| | SSRNSTPGSSRGTSPARMAGNGGDAALALLLLDRLNQLESKMSGKGQQ | |
| | QQGQTVTKKSAAEASKKPRQKRTATKAYNVTQAFGRRGPEQTQGNFGD | |
| | QELIRQGTDYKHWPQIAQFAPSASAFFGMSRIGMEVTPSGTWLTYTGA | |
| | IKLDDKDPNFKDQVILLNKHIDAYKTFPPTEPKKDKKKKADETQALPQ | |
| | RQKKQQTVTLLPAADLDDFSKQLQQSMSSADSTQA | |
| 49 | atgtgagcaaaaggccagcaaaaggccagggaccgtaaaaaggccgcg | DNA sequence of pFRG- |
| | ttgctggcgtttttccataggctccgcccccctgacgagcatcacaaa | CMV-Sgene (artificial) |
| | aatcgacgctcaagtcagaggtggcgaaacccgacaggactataaaga | E. coli Origin of |
| | taccaggcgtttccccctggaagctccctcgtgcgctctcctgttccg | Replication 1-692 bp |
| | accctgccgcttaccggatacctgtccgcctttctcccttcgggaagc | Spectinomycin Resistance |
| | gtggcgctttctcatagctcacgctgtaggtatctcagttcggtgtag | Cassette 699-1734 bp |
| | gtcgttcgctccaagctgggctgtgtgcacgaaccccccgttcagccc | CMV Enhancer/Promoter |
| | gaccgctgcgccttatccggtaactatcgtcttgagtccaacccggta | sequence 1741-2467 bp |
| | agacacgacttatcgccactggcagcagccactggtaacaggattagc | [CMV Enhancer |
| | agagcgaggtatgtaggcggtgctacagagttcttgaagtggtggcct | 1791-2165 bp] |
| | aactacggctacactagaagaacagtatttggtatctgcgctctgctg | [CMV Promoter |
| | aagccagttaccttcggaaaaagagttggtagctcttgatccggcaaa | 2166-2385 bp] |
| | caaaccaccgctggtagcggtggtttttttgtttgcaagcagcagatt | [CMV 5′UTR |
| | acgcgcagaaaaaaaggatctcaagaagatcctttgatcttttctacg | 2386-2457 bp] |
| | ggtttttttggggcggctttgaattcttttttttggggcggctttttt | S Gene (Spike) {SARS- |
| | tttatgcgctcacgcaactggtccagaaccttgaccgaacgcagcggt | CoV-2} 2478-6299 bp |
| | ggtaacggcgcagtggcggttttcatggcttgttatgactgttttttt | HSV-TK PolyA Signal |
| | ggggtacagtctatgcctcgggcatccaagcagcaagcgcgttacgcc | 6300-6620 bp |
| | gtgggtcgatgtttgatgttatggagcagcaacgatgttacgcagcag | Bacterial Rb Promoter |
| | ggcagtcgccctaaaacaaagttaaacatcatgagggaagcggtgatc | 6627-6700 bp |
| | gccgaagtatcgactcaactatcagaggtagttggcgtcatcgagcgc | Hybrid Transport Protein |
| | catctcgaaccgacgttgctggccgtacatttgtacggctccgcagtg | 6701-7102 bp |
| | gatggcggcctgaagccacacagtgatattgatttgctggttacggtg | Secretion Signal |
| | accgtaaggcttgatgaaacaacgcggcgagctttgatcaacgacctt | 6701-6790 bp] |
| | ttggaaacttcggcttcccctggagagagcgagattctccgcgctgta | [DNA Binding Domain |
| | gaagtcaccattgttgtgcacgacgacatcattccgtggcgttatcca | 6791-7066 bp] |
| | gctaagcgcgaactgcaatttggagaatggcagcgcaatgacattctt | [CPP 7067-7102 bp] |
| | gcaggtatcttcgagccagccacgatcgacattgatctggctatcttg | SynS Terminator 7103- |
| | ctgacaaaagcaagagaacatagcgttgccttggtaggtccagcggcg | 7144 bp |
| | gaggaactctttgatccggttcctgaacaggatctatttgaggcgcta | Bif Origin of Replication |
| | aatgaaaccttaacgctatggaactcgccgcccgactgggctggcgat | 7151-8978 bp |
| | gagcgaaatgtagtgcttacgttgtcccgcatttggtacagcgcagta | |
| | accggcaaaatcgcgccgaaggatgtcgctgccgactgggcaatggag | |
| | cgcctgccggcccagtatcagcccgtcatacttgaagctagacaggct | |
| | tatcttggacaagaagaagatcgcttggcctcgcgcgcagatcagttg | |
| | gaagaatttgtccactacgtgaaaggcgagatcaccaaggtagtcggc | |
| | aaataaggtaccgttaggcgttttcgcgatgtacgggccagatatacg | |
| | cgttgacattgattattgactagttattaatagtaatcaattacgggg | |
| | tcattagttcatagcccatatatggagttccgcgttacataacttacg | |
| | gtaaatggcccgcctggctgaccgcccaacgacccccgcccattgacg | |
| | tcaataatgacgtatgttcccatagtaacgccaatagggactttccat | |
| | tgacgtcaatgggtggagtatttacggtaaactgcccacttggcagta | |
| | catcaagtgtatcatatgccaagtacgccccctattgacgtcaatgac | |
| | ggtaaatggcccgcctggcattatgcccagtacatgaccttatgggac | |
| | tttcctacttggcagtacatctacgtattagtcatcgctattaccatg | |
| | gtgatgcggttttggcagtacatcaatgggcgtggatagcggtttgac | |
| | tcacggggatttccaagtctccaccccattgacgtcaatgggagtttg | |
| | ttttggcaccaaaatcaacgggactttccaaaatgtcgtaacaactcc | |
| | gccccattgacgcaaatgggcggtaggcgtgtacggtgggaggtctat | |
| | ataagcagagctcgtttagtgaaccgtcagatcgcctggagacgccat | |
| | ccacgctgttttgacctccatagaagacaccgggaccgatccagcctc | |
| | cggactctagaggatcgaagctagccaccatgtttgtttttcttgttt | |
| | tattgccactagtctctagtcagtgtgttaatcttacaaccagaactc | |
| | aattaccccctgcatacactaattctttcacacgtggtgtttattacc | |
| | ctgacaaagttttcagatcctcagttttacattcaactcaggacttgt | |
| | tcttacctttcttttccaatgttacttggttccatgctatacatgtct | |
| | ctgggaccaatggtactaagaggtttgataaccctgtcctaccattta | |
| | atgatggtgtttattttgcttccactgagaagtctaacataataagag | |
| | gctggatttttggtactactttagattcgaagacccagtccctactta | |
| | ttgttaataacgctactaatgttgttattaaagtctgtgaatttcaat | |
| | tttgtaatgatccatttttgggtgtttattaccacaaaaacaacaaaa | |
| | gttggatggaaagtgagttcagagtttattctagtgcgaataattgca | |
| | cttttgaatatgtctctcagccttttcttatggaccttgaaggaaaac | |
| | agggtaatttcaaaaatcttagggaatttgtgtttaagaatattgatg | |
| | gttattttaaaatatattctaagcacacgcctattaatttagtgcgtg | |
| | atctccctcagggtttttcggctttagaaccattggtagatttgccaa | |
| | taggtattaacatcactaggtttcaaactttacttgctttacatagaa | |
| | gttatttgactcctggtgattcttcttcaggttggacagctggtgctg | |
| | cagcttattatgtgggttatcttcaacctaggacttttctattaaaat | |
| | ataatgaaaatggaaccattacagatgctgtagactgtgcacttgacc | |
| | ctctctcagaaacaaagtgtacgttgaaatccttcactgtagaaaaag | |
| | gaatctatcaaacttctaactttagagtccaaccaacagaatctattg | |
| | ttagatttcctaatattacaaacttgtgcccttttggtgaagttttta | |
| | acgccaccagatttgcatctgtttatgcttggaacaggaagagaatca | |
| | gcaactgtgttgctgattattctgtcctatataattccgcatcatttt | |
| | ccacttttaagtgttatggagtgtctcctactaaattaaatgatctct | |
| | gctttactaatgtctatgcagattcatttgtaattagaggtgatgaag | |
| | tcagacaaatcgctccagggcaaactggaaagattgctgattataatt | |
| | ataaattaccagatgattttacaggctgcgttatagcttggaattcta | |
| | acaatcttgattctaaggttggtggtaattataattacctgtatagat | |
| | tgtttaggaagtctaatctcaaaccttttgagagagatatttcaactg | |
| | aaatctatcaggccggtagcacaccttgtaatggtgttgaaggtttta | |
| | attgttactttcctttacaatcatatggtttccaacccactaatggtg | |
| | ttggttaccaaccatacagagtagtagtactttcttttgaacttctac | |
| | atgcaccagcaactgtttgtggacctaaaaagtctactaatttggtta | |
| | aaaacaaatgtgtcaatttcaacttcaatggtttaacaggcacaggtg | |
| | ttcttactgagtctaacaaaaagtttctgcctttccaacaatttggca | |
| | gagacattgctgacactactgatgctgtccgtgatccacagacacttg | |
| | agattcttgacattacaccatgttcttttggtggtgtcagtgttataa | |
| | caccaggaacaaatacttctaaccaggttgctgttctttatcaggatg | |
| | ttaactgcacagaagtccctgttgctattcatgcagatcaacttactc | |
| | ctacttggcgtgtttattctacaggttctaatgtttttcaaacacgtg | |
| | caggctgtttaataggggctgaacatgtcaacaactcatatgagtgtg | |
| | acatacccattggtgcaggtatatgcgctagttatcagactcagacta | |
| | attctcctcggcgggcacgtagtgtagctagtcaatccatcattgcct | |
| | acactatgtcacttggtgcagaaaattcagttgcttactctaataact | |
| | ctattgccatacccacaaattttactattagtgttaccacagaaattc | |
| | taccagtgtctatgaccaagacatcagtagattgtacaatgtacattt | |
| | gtggtgattcaactgaatgcagcaatcttttgttgcaatatggcagtt | |
| | tttgtacacaattaaaccgtgctttaactggaatagctgttgaacaag | |
| | acaaaaacacccaagaagtttttgcacaagtcaaacaaatttacaaaa | |
| | caccaccaattaaagattttggtggttttaatttttcacaaatattac | |
| | cagatccatcaaaaccaagcaagaggtcatttattgaagatctacttt | |
| | tcaacaaagtgacacttgcagatgctggcttcatcaaacaatatggtg | |
| | attgccttggtgatattgctgctagagacctcatttgtgcacaaaagt | |
| | ttaacggccttactgttttgccacctttgctcacagatgaaatgattg | |
| | ctcaatacacttctgcactgttagcgggtacaatcacttctggttgga | |
| | cctttggtgcaggtgctgcattacaaataccatttgctatgcaaatgg | |
| | cttataggtttaatggtattggagttacacagaatgttctctatgaga | |
| | accaaaaattgattgccaaccaatttaatagtgctattggcaaaattc | |
| | aagactcactttcttccacagcaagtgcacttggaaaacttcaagatg | |
| | tggtcaaccaaaatgcacaagctttaaacacgcttgttaaacaactta | |
| | gctccaattttggtgcaatttcaagtgttttaaatgatatcctttcac | |
| | gtcttgacaaagttgaggctgaagtgcaaattgataggttgatcacag | |
| | gcagacttcaaagtttgcagacatatgtgactcaacaattaattagag | |
| | ctgcagaaatcagagcttctgctaatcttgctgctactaaaatgtcag | |
| | agtgtgtacttggacaatcaaaaagagttgatttttgtggaaagggct | |
| | atcatcttatgtccttccctcagtcagcacctcatggtgtagtcttct | |
| | tgcatgtgacttatgtccctgcacaagaaaagaacttcacaactgctc | |
| | ctgccatttgtcatgatggaaaagcacactttcctcgtgaaggtgtct | |
| | ttgtttcaaatggcacacactggtttgtaacacaaaggaatttttatg | |
| | aaccacaaatcattactacagacaacacatttgtgtctggtaactgtg | |
| | atgttgtaataggaattgtcaacaacacagtttatgatcctttgcaac | |
| | ctgaattagactcattcaaggaggagttagataaatattttaagaatc | |
| | atacatcaccagatgttgatttaggtgacatctctggcattaatgctt | |
| | cagttgtaaacattcaaaaagaaattgaccgcctcaatgaggttgcca | |
| | agaatttaaatgaatctctcatcgatctccaagaacttggaaagtatg | |
| | agcagtatataaaatggccatggtacatttggctaggttttatagctg | |
| | gcttgattgccatagtaatggtgacaattatgctttgctgtatgacca | |
| | gttgctgtagttgtctcaagggctgttgttcttgtggatcctgctgca | |
| | aatttgatgaagacgactctgagccagtgctcaaaggagtcaaattac | |
| | attacacataatcgatccctaccggttagtaatgagtttaaacggggg | |
| | aggctaactgaaacacggaaggagacaataccggaaggaacccgcgct | |
| | atgacggcaataaaaagacagaataaaacgcacgggtgttgggtcgtt | |
| | tgttcataaacgcggggttcggtcccagggctggcactctgtcgatac | |
| | cccaccgagaccccattggggccaatacgcccgcgtttcttccttttc | |
| | cccaccccaccccccaagttcgggtgaaggcccagggctcgcagccaa | |
| | cgtcggggcggcaggccctgccatagctttttggggcggcttttctcg | |
| | agtccttccttaaggacgtgctcgtcaatttttgttttgagagtcatc | |
| | tattcggatgcttttcatgaagttttttatgaccctgaccggcaccct | |
| | gcgcaaggccttcgccaccaccctggccgccgccatgctgatcggcac | |
| | cctggccggctgctcctccgccgcatacaacaagtctgacctcgtttc | |
| | gaagatcgcccagaagtccaacctgaccaaggctcaggccgaggctgc | |
| | tgttaacgccttccaggatgtgttcgtcgaggctatgaagtccggcga | |
| | aggcctgaagctcaccggcctgttctccgctgagcgcgtcaagcgccc | |
| | ggctcgcaccggccgcaacccgcgcactggcgagcagattgacattcc | |
| | ggcttcctacggcgttcgtatctccgctggctccctgctgaagaaggc | |
| | cgtcaccgagtatggacggaagaagcgcaggcagcgacggcgatgagg | |
| | gtttgcgcttgcgtcgtggagggagcggaacgccgaaaaaggatccac | |
| | gcgctggagatgttcaacgagtagatcgccacggcgacctccttccac | |
| | gcgtgcgggcacggggattctcaaggggccggcccgaggccccttgag | |
| | cccgccgggaggcgcccccggcagggcgggaatccaaagggcggagcc | |
| | ctgtggccctccccgggcaggggcgggatcgtcaagggcggagccctt | |
| | ggccccctcgggagagcgcactgacacaatgctacctccggtagcatt | |
| | aagtgcgccctccgccatgcggagggacgggccgcgaccggatatgcg | |
| | gggaacgtccacgacgcgtcttccgtgtcctccgtcctgccttgtgcc | |
| | gtcgattataatcctcggataacggacgatgattgaaccgattggagg | |
| | aaacgagatgccgaagagtttcgcgcagcagatcgaggacgacgagaa | |
| | caagatcaagcgcatccgggagcaccagcgcatggtcagggccaaaca | |
| | agccaaacaggagcgcaacgcccgcaccaaaaggctcgtggagaccgg | |
| | agcgatagtggaaaaagcgcacggcggcgcgtacgacgacgaaggacg | |
| | gcagacgttctcggatggtctgaacggcatcatctcggtctacgaccc | |
| | gtcccgcggcggcaacgtggacatgagagtcatcgacgtaatcgaccg | |
| | gcgcatccccagattgccaaggtccgaaaccacaacaggcacggcagc | |
| | ggcggcatcgcgaaccgtgcaagccaccgcaccacagccagcccacgc | |
| | ccaaccgcaaagcttcacgcccaacccccagcgcatcgaacaccggac | |
| | aggagcacagcagccggatcggtgggcctagccgggacggcgcagccc | |
| | tcgctccgctgggctgaaatatctgacttggttcgtaacatatttccg | |
| | acatgtcggaaatatgtcgtatcatcgttgctatgagtactgaacttc | |
| | gtgagcagtgggagcagctttacctgccgctgcgcccgctgtgcacga | |
| | acgacttcatcgagggcgtgtaccggcagcccagggcgaaggcgctgg | |
| | agggctaccggtacatcgaggcgaaccccaagaccgtcagcgacctgc | |
| | tcgtggtcgacatcgacgacgcgaacgcccgcgcgatggccctatggg | |
| | agcatgaggggatgctgcccaacgtgatcgtggagaacccgagaaacg | |
| | gccacgcgcacgccgtgtgggcgctggccgccccgttcagtcgcaccg | |
| | aatacgcgcggcgcaagccgctcgccctggccgcggcggtcacggagg | |
| | ggctgcgccgctcgtgcgacggggacaagggatattcggggctgatga | |
| | ccaagaacccgctgcacgaggcgtggaacagcgaactggtcaccgatc | |
| | acctgtacgggctggacgaactgcgcgaggcgctggaggagtccgggg | |
| | acatgccgccggcctcgtggaagcgcacgaaacggcgcaacgtggtcg | |
| | gattgggccgcaactgccatctgttcgagacggcgcgcacatgggcgt | |
| | acagggaggtgcgccaccactggggagacagcgaggggctgcggctgg | |
| | ccatctcggccgaggcgcacgaaatgaacgccgaactgttccccgagc | |
| | cactggcatggtcggaggtcgagcagatcgccaagagcatccacaagt | |
| | ggatcgtcacccaaagccgcatgtggcgcgacggccccaccgtctacg | |
| | aggcgacattcatcaccgtccaaagcgcccgagggaaaaagtcaggca | |
| | aggccagacgagaaacattcgaactattcgcgagcgaggagatgacgt | |
| | aa | |
| 50 | RVQPTESIVRFPNITNLCPFGEVFNATRFASVYAWNRKRISNCVADYS | SARS-CoV-2 (wildtype) |
| | VLYNSASFSTFKCYGVSPTKLNDLCFTNVYADSFVIRGDEVRQIAPGQ | spike Receptor Binding |
| | TGKIADYNYKLPDDFTGCVIAWNSNNLDSKVGGNYNYLYRLFRKSNLK | Domain |
| | PFERDISTEIYQAGSTPCNGVEGFNCYFPLQSYGFQPTNGVGYQPYRV | |
| | VVLSFELLHAPATVCGPKKSTNLVKNKCVNF | |
| 51 | NSNNLDSKVGGNYNYLYRLERKSNLKPFERDISTEIYQAGSTPCNGVE | SARS-CoV-2 (wildtype) |
| | GFNCYFPLQSYGFQPTNGVGYQPY | spike Receptor Binding |
| | | Motif |
| 52 | SQCVNLTTRTQLPPAYTNSFTRGVYYPDKVFRSSVLHSTQDLFLPFFS | SARS-Cov-2 (wildtype) |
| | NVTWFHAIHVSGTNGTKRFDNPVLPFNDGVYFASTEKSNIIRGWIFGT | spike protein without |
| | TLDSKTQSLLIVNNATNVVIKVCEFQFCNDPFLGVYYHKNNKSWMESE | signal peptide/secretion |
| | FRVYSSANNCTFEYVSQPFLMDLEGKQGNFKNLREFVFKNIDGYFKIY | signal |
| | SKHTPINLVRDLPQGFSALEPLVDLPIGINITRFQTLLALHRSYLTPG | |
| | DSSSGWTAGAAAYYVGYLQPRTFLLKYNENGTITDAVDCALDPLSETK | |
| | CTLKSFTVEKGIYQTSNFRVQPTESIVRFPNITNLCPFGEVFNATRFA | |
| | SVYAWNRKRISNCVADYSVLYNSASFSTFKCYGVSPTKLNDLCFTNVY | |
| | ADSFVIRGDEVRQIAPGQTGKIADYNYKLPDDFTGCVIAWNSNNLDSK | |
| | VGGNYNYLYRLFRKSNLKPFERDISTEIYQAGSTPCNGVEGFNCYFPL | |
| | QSYGFQPTNGVGYQPYRVVVLSFELLHAPATVCGPKKSTNLVKNKCVN | |
| | FNFNGLTGTGVLTESNKKFLPFQQFGRDIADTTDAVRDPQTLEILDIT | |
| | PCSFGGVSVITPGTNTSNQVAVLYQDVNCTEVPVAIHADQLTPTWRVY | |
| | STGSNVFQTRAGCLIGAEHVNNSYECDIPIGAGICASYQTQTNSPRRA | |
| | RSVASQSIIAYTMSLGAENSVAYSNNSIAIPTNFTISVTTEILPVSMT | |
| | KTSVDCTMYICGDSTECSNLLLQYGSFCTQLNRALTGIAVEQDKNTQE | |
| | VFAQVKQIYKTPPIKDFGGFNFSQILPDPSKPSKRSFIEDLLFNKVTL | |
| | ADAGFIKQYGDCLGDIAARDLICAQKFNGLTVLPPLLTDEMIAQYTSA | |
| | LLAGTITSGWTFGAGAALQIPFAMQMAYRFNGIGVTQNVLYENQKLIA | |
| | NQFNSAIGKIQDSLSSTASALGKLQDVVNQNAQALNTLVKQLSSNFGA | |
| | ISSVLNDILSRLDKVEAEVQIDRLITGRLQSLQTYVTQQLIRAAEIRA | |
| | SANLAATKMSECVLGQSKRVDFCGKGYHLMSFPQSAPHGVVFLHVTYV | |
| | PAQEKNFTTAPAICHDGKAHFPREGVFVSNGTHWFVTQRNFYEPQIIT | |
| | TDNTFVSGNCDVVIGIVNNTVYDPLQPELDSFKEELDKYFKNHTSPDV | |
| | DLGDISGINASVVNIQKEIDRLNEVAKNLNESLIDLQELGKYEQYIKW | |
| | PWYIWLGFIAGLIAIVMVTIMLCCMTSCCSCLKGCCSCGSCCKEDEDD | |
| | SEPVLKGVKLHYT | |
| 53 | MFVFLVLLPLVSSQCVNFTTRTQLPPAYTNSFTRGVYYPDKVFRSSVL | SARS-CoV-2 (variant |
| | HSTQDLFLPFFSNVTWFHAIHVSGTNGTKRFANPVLPFNDGVYFASTE | B.1.351) spike protein |
| | KSNIIRGWIFGTTLDSKTQSLLIVNNATNVVIKVCEFQFCNDPFLGVY | Signal Peptide/Secretion |
| | YHKNNKSWMESEFRVYSSANNCTFEYVSQPFLMDLEGKQGNFKNLREF | Signal 1-12 aa |
| | VFKNIDGYFKIYSKHTPINLVRGLPQGFSALEPLVDLPIGINITRFQT | S1 domain 13-682 aa |
| | LHRSYLTPGDSSSGWTAGAAAYYVGYLQPRTFLLKYNENGTITDAVDC | (cleavage site at R682- |
| | ALDPLSETKCTLKSFTVEKGIYQTSNERVQPTESIVRFPNITNLCPFG | S683) |
| | EVFNATRFASVYAWNRKRISNCVADYSVLYNSASFSTFKCYGVSPTKL | S2 domain 683-1270 aa |
| | NDLCFTNVYADSFVIRGDEVRQIAPGQTGNIADYNYKLPDDFTGCVIA | (cleavage site at R812- |
| | WNSNNLDSKVGGNYNYLYRLFRKSNLKPFERDISTEIYQAGSTPCNGV | S813) |
| | KGFNCYFPLQSYGFQPTYGVGYQPYRVVVLSFELLHAPATVCGPKKST | S2′ domain 813-1270 aa |
| | NLVKNKCVNFNFNGLTGTGVLTESNKKELPFQQFGRDIADTTDAVRDP | Receptor Binding Domain |
| | QTLEILDITPCSFGGVSVITPGTNTSNQVAVLYQGVNCTEVPVAIHAD | 316-538 aa |
| | QLTPTWRVYSTGSNVFQTRAGCLIGAEHVNNSYECDIPIGAGICASYQ | Receptor Binding Motif |
| | TQTNSPRRARSVASQSIIAYTMSLGVENSVAYSNNSIAIPTNFTISVT | 434-505 aa |
| | TEILPVSMTKTSVDCTMYICGDSTECSNLLLQYGSFCTQLNRALTGIA | Transmembrane Domain |
| | VEQDKNTQEVFAQVKQIYKTPPIKDFGGFNFSQILPDPSKPSKRSFIE | 1211-1231 aa |
| | DLLFNKVTLADAGFIKQYGDCLGDIAARDLICAQKFNGLTVLPPLLTD | Cytoplasmic Domain |
| | EMIAQYTSALLAGTITSGWTFGAGAALQIPFAMQMAYRFNGIGVTQNV | 1242-1270 aa |
| | LYENQKLIANQFNSAIGKIQDSLSSTASALGKLQDVVNQNAQALNTLV | |
| | KQLSSNFGAISSVLNDILSRLDKVEAEVQIDRLITGRLQSLQTYVTQQ | |
| | LIRAAEIRASANLAATKMSECVLGQSKRVDFCGKGYHLMSFPQSAPHG | |
| | VVFLHVTYVPAQEKNFTTAPAICHDGKAHFPREGVFVSNGTHWFVTQR | |
| | NFYEPQIITTDNTFVSGNCDVVIGIVNNTVYDPLQPELDSFKEELDKY | |
| | FKNHTSPDVDLGDISGINASVVNIQKEIDRLNEVAKNLNESLIDLQEL | |
| | GKYEQYIKWPWYIWLGFIAGLIAIVMVTIMLCCMTSCCSCLKGCCSCG | |
| | SCCKFDEDDSEPVLKGVKLHYT | |
| 54 | RVQPTESIVRFPNITNLCPFGEVFNATRFASVYAWNRKRISNCVADYS | SARS-CoV-2 spike |
| | VLYNSASFSTFKCYGVSPTKLNDLCFTNVYADSFVIRGDEVRQIAPGQ | (variant B.1.351) |
| | TGNIADYNYKLPDDFTGCVIAWNSNNLDSKVGGNYNYLYRLFRKSNLK | Receptor Binding Domain |
| | PFERDISTEIYQAGSTPCNGVKGFNCYFPLQSYGFQPTYGVGYQPYRV | |
| | VVLSFELLHAPATVCGPKKSTNLVKNKCVNF | |
| 55 | SNNLDSKVGGNYNYLYRLFRKSNLKPFERDISTEIYQAGSTPCNGVKG | SARS-CoV-2 spike |
| | FNCYFPLQSYGFQPTYGVGYQPY | (variant B.1.351) |
| | | Receptor Binding Motif |
| 56 | SQCVNFTTRTQLPPAYTNSFTRGVYYPDKVFRSSVLHSTQDLFLPFFS | SARS-Cov-2 (variant |
| | NVTWFHAIHVSGTNGTKRFANPVLPFNDGVYFASTEKSNIIRGWIFGT | B.1.351) spike protein |
| | TLDSKTQSLLIVNNATNVVIKVCEFQFCNDPFLGVYYHKNNKSWMESE | without signal |
| | FRVYSSANNCTFEYVSQPFLMDLEGKQGNFKNLREFVFKNIDGYFKIY | peptide/secretion signal |
| | SKHTPINLVRGLPQGFSALEPLVDLPIGINITRFQTLHRSYLTPGDSS | |
| | SGWTAGAAAYYVGYLQPRTELLKYNENGTITDAVDCALDPLSETKCTL | |
| | KSFTVEKGIYQTSNFRVQPTESIVRFPNITNLCPFGEVFNATRFASVY | |
| | AWNRKRISNCVADYSVLYNSASFSTFKCYGVSPTKLNDLCFTNVYADS | |
| | FVIRGDEVRQIAPGQTGNIADYNYKLPDDFTGCVIAWNSNNLDSKVGG | |
| | NYNYLYRLFRKSNLKPFERDISTEIYQAGSTPCNGVKGFNCYFPLQSY | |
| | GFQPTYGVGYQPYRVVVLSFELLHAPATVCGPKKSTNLVKNKCVNFNF | |
| | NGLTGTGVLTESNKKELPFQQFGRDIADTTDAVRDPQTLEILDITPCS | |
| | FGGVSVITPGTNTSNQVAVLYQGVNCTEVPVAIHADQLTPTWRVYSTG | |
| | SNVFQTRAGCLIGAEHVNNSYECDIPIGAGICASYQTQTNSPRRARSV | |
| | ASQSIIAYTMSLGVENSVAYSNNSIAIPTNFTISVTTEILPVSMTKTS | |
| | VDCTMYICGDSTECSNLLLQYGSFCTQLNRALTGIAVEQDKNTQEVFA | |
| | QVKQIYKTPPIKDFGGFNFSQILPDPSKPSKRSFIEDLLFNKVTLADA | |
| | GFIKQYGDCLGDIAARDLICAQKFNGLTVLPPLLTDEMIAQYTSALLA | |
| | GTITSGWTFGAGAALQIPFAMQMAYRFNGIGVTQNVLYENQKLIANQF | |
| | NSAIGKIQDSLSSTASALGKLQDVVNQNAQALNTLVKQLSSNFGAISS | |
| | VLNDILSRLDKVEAEVQIDRLITGRLQSLQTYVTQQLIRAAEIRASAN | |
| | LAATKMSECVLGQSKRVDFCGKGYHLMSFPQSAPHGVVFLHVTYVPAQ | |
| | EKNFTTAPAICHDGKAHFPREGVFVSNGTHWFVTQRNFYEPQIITTDN | |
| | TFVSGNCDVVIGIVNNTVYDPLQPELDSFKEELDKYFKNHTSPDVDLG | |
| | DISGINASVVNIQKEIDRLNEVAKNLNESLIDLQELGKYEQYIKWPWY | |
| | IWLGFIAGLIAIVMVTIMLCCMTSCCSCLKGCCSCGSCCKFDEDDSEP | |
| | VLKGVKLHYT | |

The present invention is further illustrated by the following examples.

### IV. Examples

The term “bacTRL” generically refers to a bacteria host cell containing a plasmid (as disclosed herein) which encodes for expression of a transporter polypeptide in bacteria and for expression of a payload in eukaryotic cells.

### Example 1. Oral Administration, Delivery of Luciferase Gene into Colon Epithelial Cells and Secretion of Luciferase into Bloodstream

This Example confirms that bacTRL-GLuc, a bacteria designed to deliver a plasmid encoding the Gaussia luciferase (GLuc) gene into mammalian cells, colonizes the colon (large intestine) and causes secretion of GLuc as a payload/reporter protein by cells of the large intestine into blood via the colon. The bacTRL-GLuc bacteria was prepared by transforming Bifidobacterium longum subsp. longum. with plasmid pFRG3.5-CMV-GLuc (SEQ ID NO:43; see Table 1) using known transformation protocols (e.g. see PCT Publication Nos. WO/2015/120541 and WO/2015/120542). Eight (8) mice (female C57BL/6) were dosed with bacTRL-GLuc (200 μL of 10⁹ CFU total oral gavage administration; formulated in PBS pH 7.4+10% w/v Sucrose) once daily for 12 days. The samples of bacTRL-GLuc were thawed from frozen, at room temperature for 5-6 minutes immediately prior to oral gavage. Four (4) control mice (female C57BL/6) were dosed with saline once daily for 12 days. All animals were dosed daily with 500 μL of 40% lactulose via IP.

### 1.1 Systemic GLuc Levels

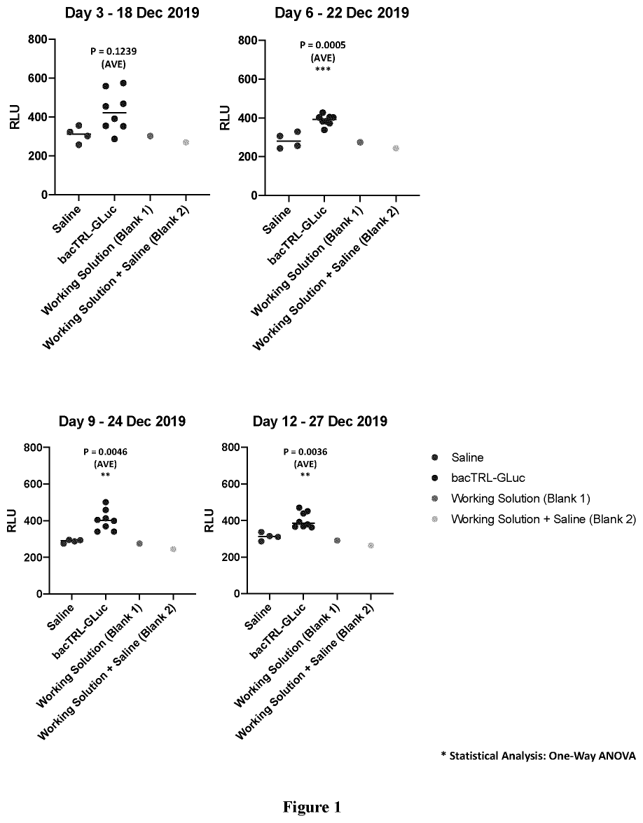

To evaluate circulating GLuc levels, 50 μL blood was obtained from each mouse on Days 3, 6, 9 and 12 (sacrifice day) and added to 10 μL 20 mM EDTA (5:1 ratio). GLuc was measured using the commercially available kit “Pierce Gaussia Luicerase Glow Assay Kit (#16160, Thermo Scientific); also see Wurdinger et al. (2008) Nature Methods 5(2):171-173. For example: 50 μL of blood or serum was added to 125 μL of working solution (GLuc assay buffer+Coelenterazine) and photon counts were acquired at 485 nm for 10 seconds. Total relative light units (RLU) per second were recorded. Systemic delivery of GLuc was estimated as RLU (bacTRL-GLuc treated)-RLU (Baseline or Saline control). The results are shown graphically in FIG. 1, which shows that increased fluorescent signal (from the presence of GLuc) was detected in the blood of all mice treated orally with bacTRL-GLuc at all time points in the study. Furthermore, an increase in statistical significance was observed at later time points, indicating stable GI colonization and steady gene delivery, secretion and systemic availability.

1.2 In Vivo bacTRL-GLuc Colonization of the Colon

All mice were sacrificed at the end of the 12 days.

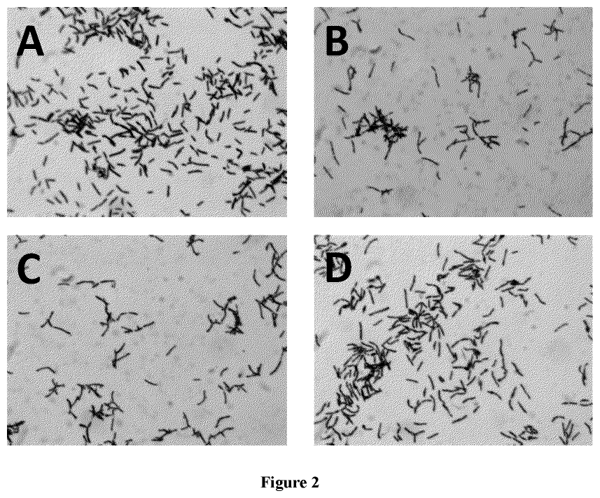

Two (2) saline colons and four (4) bacTRL-GLuc colons were harvested with luminal constituents intact and flash frozen in liquid nitrogen. The colons were then homogenized and assayed for extant bacTRL-GLuc colonization. Briefly, whole colon homogenates were processed using Qiagen Tissue Lyser, incubated anaerobically for 2-3 days at 37 degrees celsius and plated on RCA plates supplemented with 250 uL spectinomycin to select for transformed bacTRL-GLuc growth. Colonies formed were quantitated and total CFU per gram of colonic tissue was determined. Each biological replicate has 3 technical replicates based on sample volume and availability. The final CFU per mL and CFU per g of colon tissue is reported in Table 2 (below), and shows that bacTRL-GLuc colonization viable counts were obtained between 1.2 to 4.8×10⁵ CFU per g for all the tissue homogenates, except for M5, which showed a lower colonization count of 9.4×10² of total CFU's per g of tissue used. As shown in FIG. 2, Gram staining the colonies from the M5 (A), M6 (B), M7 (C) and M8 (D) bacTRL-GLuc treated colons confirmed that the bacteria were characteristic of B. longum cell morphology (Gram-positive violet-colored, rod shaped bacteria, in short chains, bifurcations, and short branches, sometimes scattered).

TABLE 2
Summary of CFU analysis using CFU per g of whole colon
tissue from the end-point samples of GI study to determine
selective bacTRL-GLuc colonization in the colon.

| Subject Study Mouse Designation | Cell count | |
|---|---|---|
| (Whole colon tissue homogenate) | (CFU per g of GI Tissue) | |
| bacTLR-GLuc (M5) | 9.4 × 10² | CFU/g |
| bacTRL-GLuc (M6) | 4.8 × 10⁵ | CFU/g |
| bacTRL-GLuc (M7) | 1.2 × 10⁵ | CFU/g |
| bacTRL GLuc (M8) | 2.9 × 10⁵ | CFU/g |
| Saline (M1) | 0 | CFU/g |
| Saline (M2) | 0 | CFU/g |

The remaining two (2) saline colons (M3, M4) and four (4) bacTRL-GLuc colons (M9, M10, M11, and M12) were fixed in formalin and paraffin-embedded (FFPE) following standard protocol in a Swiss roll-based orientation (see Bialkowska et al. 2016 Journal of Visualized Experiments Vol. 113(e54161):1-8) and examined for bacTRL-GLuc localization and GLuc reporter gene expression within the histological landscape.

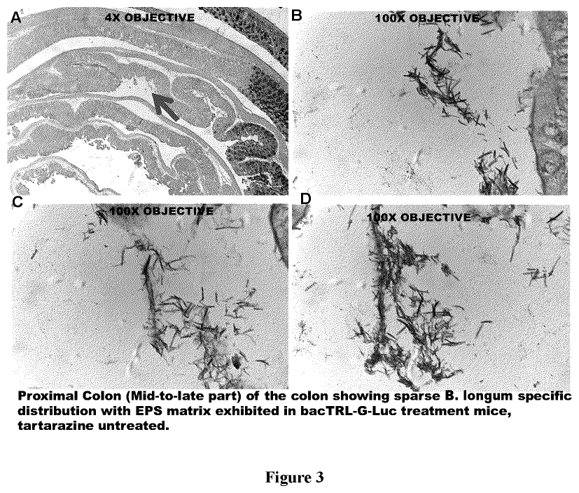

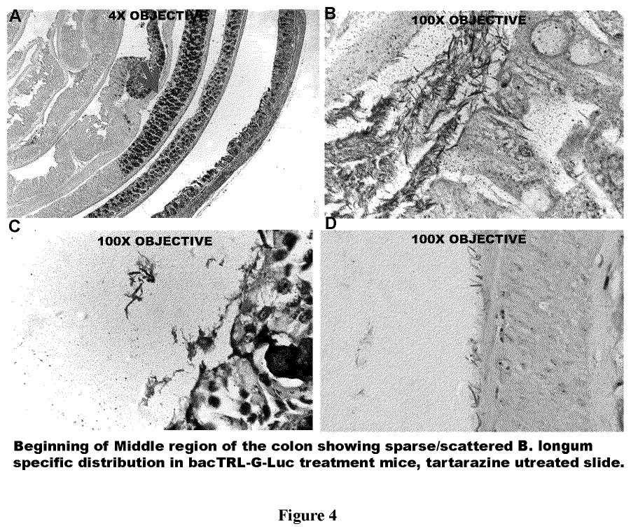

FIGS. 3 and 4 show representative gram staining of FFPE of whole colon tissue sections from the M9 bacTRL-GLuc treated mouse. Visualization was performed using Motic Panthera Trinocular microscope with Moticam S6 camera with MoticPlus 3.0 software used for capture and analysis. In FIG. 3 panel A: Proximal region of colon (mid-to-late section) showed sparse B. longum specific staining morphology without tartarazine treatment. In FIG. 3 panels B, C and D: Gram-positive rods in short chains, groups, scattered and with bifurcations (10× and 100× objective). In addition, B. longum-specific characteristic extracellular polysaccharide matrix was also observed around the bacterial cells. In FIG. 4 panel A: Beginning of middle region of colon. In FIG. 4 panels B, C and D: B. longum specific staining morphology (100× Objective) Gram-positive rods in short chains, groups, scattered and with bifurcations.

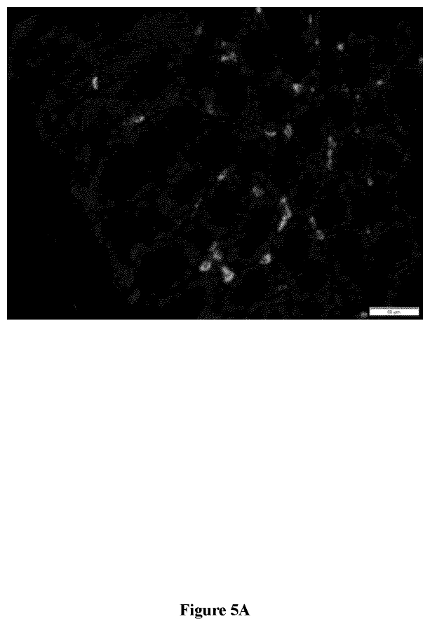

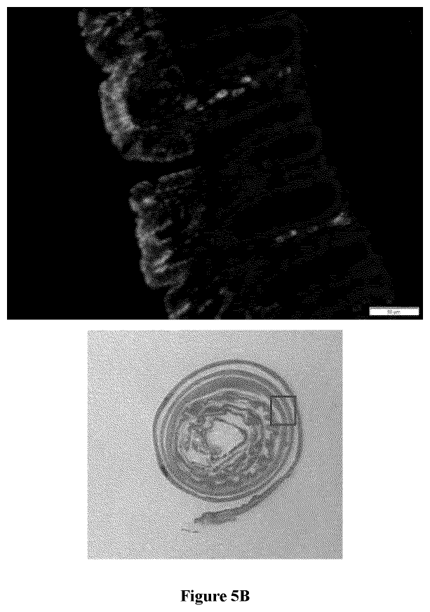

FFPE (Swiss roll preparation) colons for M3 (saline) and M 11 and M12 (bacTRL-GLuc) were also examined by immunofluorescent staining for GLuc expression using OPAL IHC Kit (NEL810001KT, PerkinElmer). Slides were deparaffinized and rehydrated. Antigen unmasking was done by microwave treatment in AR6 Buffer. Slides were cooled and blocked with PerkinElmer antibody diluent for 10 min at room temperature. Slides were probed with anti-GLuc polyclonal antibody (Invitrogen PA1-181) in PerkinElmer antibody diluent (1/400) for 2 h at room temperature. Tissues were washed in TBST and then re-probed with Opal Polymer HRP Ms+Rb for 10 min at room temperature, followed with incubation with Opal Fluorophore working solution (1/100 GFP, 520). Tissues were counterstained with DAPI (nuclei) and mounted with VectoShield. Fluorescence microscopy was used to analyze the results. Representative results are shown in FIGS. 5A (medial colon part), 5B (upper distal part) and 5C (lower distal part), magnification 20×. Exposure time was 700 ms for green channel and 30 ms for DAPI. Negative control (no primary Ab) showed no signal (data not shown).

In summary, bacTRL-GLuc, was designed to specifically deliver payload transgenes to the host gastrointestinal tract lining via oral administration of live bacteria. Periodic sampling of blood harvested from mice treated with bacTRL-GLuc daily demonstrated increasingly stable and consistent reporter gene expression and systemic availability as the treatment proceeds beyond 3 days. Robust colonization of bacTRL-GLuc was evident in all orally treated mice, specific to the lower GI tissues. A distinct pattern of G. luciferase gene delivery and expression was evident throughout the colonic landscape with more delivery to the GI lining observed in the distal portion of the large intestine. Potential staining of non-epithelial cells also detected throughout the tissue, indicating immune cells (e.g. macrophages) may have also been transfected.

### Example 2: SARS-CoV-2 DNA Vaccine for Multivalent (Spike, Matrix, and Nucleocapsid) Gene Delivery

Plasmid pFRG3.5-CMV-COVID19-SiMiN (SEQ ID NO: 45; Table 1) is a multivalent bacTRL plasmid construct designed to transport the plasmid to mammalian cells and there to express wild-type sequences for the SARS-CoV-2 Spike (S), Matrix (M) and Nucleocapsid (N) genes in sequence under control of single promoter separated by IRES sequences. Once synthesized and sequenced, the multivalent bacTRL plasmid construct will be transfected into established human cell lines to confirm appropriate transgene expression and antigen localization.

The plasmid construct will be transformed into B. longum. Transformed bacteria will be propagated and plated on antibiotic selective agar plates, allowing colony formation to occur. Individual colonies will then be selected, further propagated and analyzed through various molecular analysis techniques, confirming the presence and activity of the multivalent plasmid construct. Clones will then be propagated to create a master cell bank, which will be used for the basis of future manufacturing activities. Once the master cell bank is established, a small manufacturing run consisting of 75 doses of 10⁹ bacTRL bacteria in 200 μL of sucrose solution will be generated.

A murine study will be conducted to demonstrate the ability of bacTRL (i.e. transformed with pFRG3.5-CMV-COVID19-SiMiN) to produce an anti-SARS-CoV-2 immunological response, for example as discussed for Studies A and B below.

(Study A) A total of 11 healthy C57BL/6 mice will be used with two experimental arms. The treatment arm is comprised of 8 mice that receive an oral gavage dose of 10⁹ bacTRL-COVID-19 bacteria every second day for 14 days. The negative control arm is comprised of 3 mice that receive corresponding saline treatment for the same schedule. Blood and fecal samples will be obtained starting on day 5, and be taken every 5 days thereafter. Blood samples will undergo ELISA serological analysis to confirm the presence and kinetics of generation of neutralizing antibodies towards target S, M and N antigens. Fecal samples will undergo similar analysis for antigen specific IgA secretion. On day 40 a subset of the mice will be sacrificed, their colonic tissues excised and prepared according to histological “Swiss roll” technique, where the proximal and distal ends of the colon are rolled together enabling a single tissue section to provide molecular information along the tract of the large intestine. The sectioned tissue with then undergo molecular analysis quantifying the concentration and location of bacTRL-COVID-19 bacteria, as well as quantifying the extent and location of S, M, and N antigen expression. The remaining mice will subsequently be administered recombinant S, M and N proteins intravenously, and antibody titres will be monitored to confirm a “challenge” immunological response. The remaining mice will be sacrificed and processed in a similar manner.
(Study B) Alternatively, a total of up to 96 healthy C57BL/6 mice will be used with 12 experimental arms. Each treatment arm is comprised of 8 mice that receive a daily oral gavage dose of 10⁹ bacTRL bacteria every day for either 1 day, 3 days or 7 days. These dose regimens would constitute the priming vaccination dose. The time period to allow for immunological responses to the priming dose before further dosing will be between 14 and 28 days post-first dose. One set of treatment arms from each dose regiment will then receive a second homologous schedule of daily oral gavage of 10⁹ bacTRL bacteria every day for either 1 day, 3 days or 7 days beginning either at 14 days post-first dose, 21 days post-first dose, or 28 days post-first dose. This second dose constitutes the boost vaccination dose. The time period to allow for immunological responses to the boost dose before study endpoint will be between 42 and 56 days post-first dose. The negative control arm is comprised of 8 mice that receive corresponding saline treatment or bacTRL-GLuc for the same schedule. Blood and fecal samples will be obtained starting on day 7, and be taken every 7 days thereafter. Blood samples will undergo ELISA-based serological analysis to confirm the presence and kinetics of generation of antibodies able to specifically target S, M and N antigens. Further evaluation of these sera will be done to determine whether these samples containing SARS-CoV-2 specific antibodies are neutralizing, a measure of inhibition of viral entry into a host cell. Fecal samples will undergo similar analysis for the presence of secreted antigen specific IgA, a measure of local mucosal response against SARS-CoV-2 antigens expressed by the gut epithelia. On day of boost dosing, a subset of the mice will be sacrificed pre-boost, their colonic tissues excised and prepared according to histological “Swiss roll” technique, where the proximal and distal ends of the colon are rolled together enabling a single tissue section to provide molecular information along the tract of the large intestine. At end of study, the remaining mice will be sacrificed, their colonic tissues excised and prepared according to histological “Swiss roll” technique, where the proximal and distal ends of the colon are rolled together enabling a single tissue section to provide molecular information along the tract of the large intestine. The sectioned tissue will then undergo molecular analysis quantifying the concentration and location of bacTRL bacteria, as well as quantifying the extent and location of S, M, and N antigen expression.

Phase I trials will be performed to demonstrate the safety, immunogenicity and protective capabilities of bacTRL vaccination in healthy volunteers at meaningful risk of COVID-19 infection. Each bacTRL capsule contains 10⁹lyophilized bacteria. Cohorts of subjects will be administered one to ten capsule(s) orally with total bacTRL dose ranging from either 1×10⁹, 3×10⁹, or 1×10¹⁰. Appropriate placebo controls will be included in each cohort. Subjects will be advised on the nutritional guidelines that may aid in the establishment and maintenance of the bacterial colony. Subjects and their healthcare providers will be advised on two important contraindications, namely antibiotics treatments that will completely eliminate the bacTRL colony and additional probiotic supplements that may supplant the bacTRL colony. Oral administration of amoxicillin or erythromycin to eradicate bifidobacteria will be administered in the event of clinically meaningful toxicity.

Blood samples will be analyzed on a regular basis for neutralizing IgG antibodies as well as CD4⁺ and CD8⁺ T-cell lymphocytes specific to S, M and N antigens. Fecal samples will be analyzed to confirm the presence of the bacteria, during of colonization, and presence of neutralizing IgA antibodies specific to S, M, and N antigens. Subjects and appropriate stratified, matched controls will be followed for 12 months after vaccination to characterize the incidence of SARS-CoV-2 infections.

Upon oral administration of the bacTRL vaccine and subsequent colonic colonization and DNA delivery to human cells, the pathogen-associated antigens present in the bacterial vector will elicit an immune response, activating resident macrophages and dendritic cells in the intestine. Combined with the over-expression of the SARS-CoV-2 proteins by the host cells, the specific viral proteins and fragments will then be taken up by the activated macrophages and dendritic cells, which will then migrate to the mesenteric lymph nodes, where cognate T cells subsequently activate. Activated T cells will initiate activation of cognate B cells, facilitating B cell class switching to produce IgG and IgA, somatic hypermutation in germinal centers to select for high affinity B cell clones, and differentiation into memory B cells and plasma cells. After resolution of the response to the immunization, memory T and B cells specific to SARS-CoV-2 proteins will persist in circulation. The above Example will demonstrate the safety and efficacy of the bacTRL construct.

### Example 3: bacTRL-Spike

This example describes various pre-clinical studies of an orally administrable vaccine against SARS-CoV-2 infection that causes robust yet transient expression of the virus' spike protein in mouse colonic epithelia and mucosa. This example confirms that the spike protein is expressed in a conformation suitable for generation of protective immunity. This example demonstrates the rapid development of humoral systemic immunity, with IgG seroconversion evident at 14 days post-immunization, with levels persisting up to at least 40 days after priming dose. Development of neutralizing antibodies was observed, with serum samples from day 21 and day 40 maintaining the competitive ability to inhibit Spike binding to human ACE2 receptor. Oral administration of this vaccine, targeting antigen gene delivery to the intestinal epithelia and underlying mucosa also elicits a protective mucosal immunity, with anti-Spike IgA titers detectable in excreted fecal samples at 21 days post-vaccination.

bacTRL-Spike Construct

Plasmid pFRG-CMV-SGene (SEQ ID NO: 49; Table 1) is a monovalent bacTRL plasmid construct designed to constitutively deliver and express the full-length wild-type sequence for the SARS-CoV-2 Spike (S) gene in mammalian cells. Since the full-length spike protein sequence naturally translocates to the mammalian cell membrane, this payload protein can be detected within endosomal compartments bound to intracellular membranes as well as on the cell surface, where S1+S2 ectodomain is presented on the surface of the transfected cell. The payload also includes the spike transmembrane domain and cytoplasmic domain, and so should form an inate trimeric structure on the surface of the transfected cell. The plasmid was synthesized and transformed into Bifidobacterium longum subsp. longum. using known transformation protocols (e.g. see PCT Publication Nos. WO/2015/120541 and WO/2015/120542). The transformed bacteria is referred to herein as “bacTRL-Spike”.

### Histological Examination of Mouse Colonic Tissue Sections Using Gram Stain.

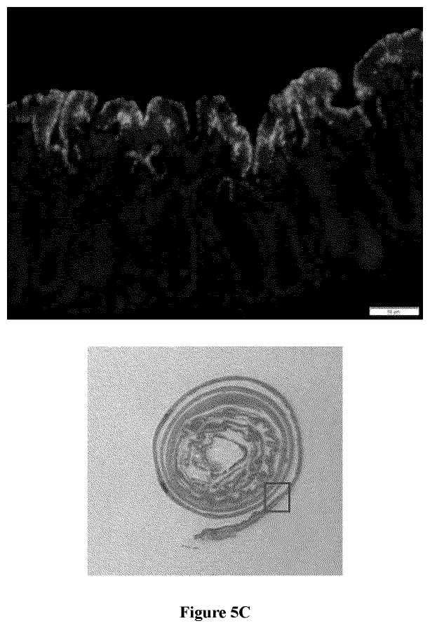

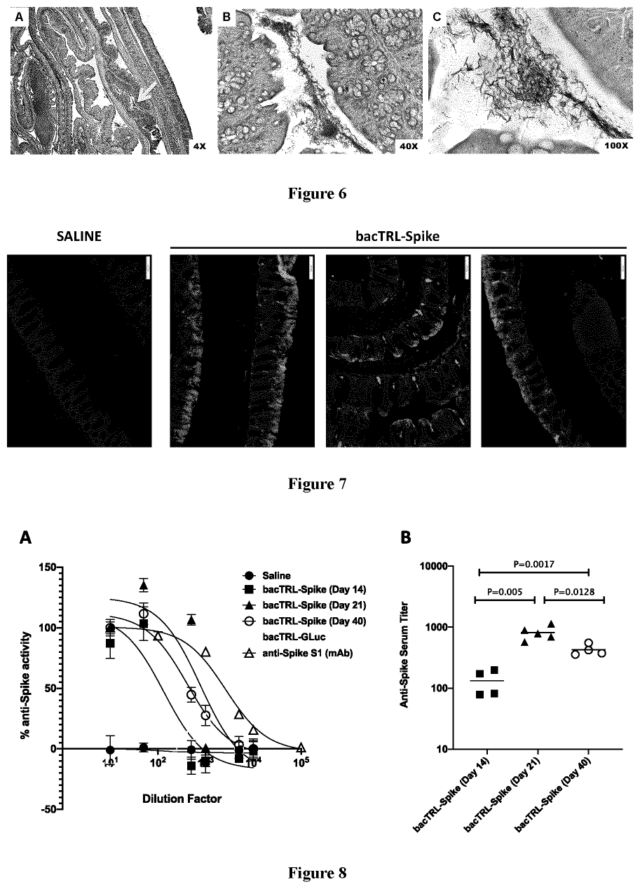

Mice (C57BL/6) were orally administered with 3 consecutive daily doses of 5×10⁸ CFU of bacTRL-Spike. The mice were harvested on day 4 and prepared for histological examination. The medial colon region was subjected to standard Gram staining and was then microscopically analyzed for the presence of Bifidobacterium longum. The results are shown in FIG. 6, where staining revealed scattered clusters of characteristic B. longum specific morphology in locations proximal and bound to the intestinal epithelial lining. FIG. 6, Panel A, shows a 4× objective magnification in which the arrow indicates a cluster of B. longum. FIG. 6, Panel B, shows a 40× objective magnification of the B. longum cluster indicated in Panel A. FIG. 6, Panel C, shows a 100× objective magnification of the B. longum cluster indicated in panel A. Key morphological parameters for visual identification included Gram-positive rods in short chains, with bifurcations in scattered/clustered groups. Visualization was performed using Motic Panthera Trinocular microscope with Moticam S6 camera with MoticPlus 3.0 software used for capture and analysis. FIG. 6 therefore confirms that oral administration of bacTRL-Spike traverses to the upper gastro-intestinal pathway until it finds the niche environment of B. longum in the lumen of the large intestine to establish robust colonization of the large intestinal lumen.

The colonization was observed to: (1) correlate with degree of oral administration, with colony forming units (CFUs) increasing with increased dose, and (2) to quickly wane once dosing is discontinued, demonstrating clearance in about 3 to 5 days after last dose.

Immunofluorescent Staining for Spike Protein Expression in Histological Colon Sections in Mice Treated Orally with bacTRL-Spike.

C57BL/6 mice were orally administered with 3 consecutive daily doses of(i) saline or (ii) 5×10⁸ CFU of bacTRL-Spike. Colons harvested from the treated mice were fixed in formalin and sectioned using standard techniques. Histological sections were processed as per standard methods for immunofluorescent detection using anti-Rabbit anti-SARS-CoV-2 (2019-nCoV) pAb (Sino Biologics 40150-R007; a human SARS Coronavirus polyclonal antibody that cross-reacts with 2019-SARS-Co-V-2 Spike). Tissues were probed with Opal Polymer HRP Ms+Rb for 10 minutes at room temperature, followed with incubation with Opal Fluorophore working solution (1/100 GFP, 520). Tissues were counterstained with DAPI (nuclei; blue) and subjected to fluorescence microscopy to analyze spike protein expression and localization (green). As shown in FIG. 7, no spike expression was observed in the colon sections of the saline-treated mice, whereas positive spike expression was observed in the colon sections of the bacTRL-Spike treated mice.

Biodistribution of both bacTRL-Spike colonization and Spike gene expression was evaluated for various non-colonic tissues (lungs & trachea, caudal esophagus, stomach, heart, kidney, liver, spleen, brain). Histological examination of these tissues revealed no evidence of bacTRL-Spike colonization nor Spike gene expression beyond the intended colonic epithelia. Tissues derived from the small Intestine (duodenum, jejunum and ileum) also did not show any positive signal in the intestinal epithelial lining.

Antibodies in Serum and Feces of bacTRL-Spike-Treated Mice Reactive to Commercial Spike Protein.

Immunogenicity of bacTRL-Spike was evaluated in C57BL/6 mice, post oral administration. 6-8 week old males and females were administered a daily dose of viable bacTRL-Spike at 5×10⁸ CFU/dose via oral gavage for 7 consecutive days. Mock vaccinations (controls) were conducted using analogous oral treatment with bacTRL-GLuc (see Example 1) or standard saline.

Serum was collected from bacTRL-Spike treated and control mice on day 14, 21 and 40 to evaluate immunogenicity of bacTRL-Spike mediated spike gene delivery to the large intestinal lining. Immunoreactivity of sera from mice immunized with bacTRL-Spike was measured using ELISA against a commercially-available trimerization-stabilized recombinant SARS-CoV-2 Spike protein construct constituting the S1+S2 ectodomain (Product #46328, LakePharma, CA, USA). FIG. 8, Panel A, shows mean (±SE) % anti-Spike immunoreactivity against recombinant trimeric Spike ectodomain (S1+S2) in serially titrated serum samples collected from bacTRL-Spike-treated mice (day 14, 21 and day 40), mock-treated bacTRL-GLuc (day 15) and saline-treated mice (day 21). The mean (±SE) % anti-Spike binding activity of a commercially available anti-SARS-CoV-2 Spike S1 antibody (rabbit monoclonal, Sino Biological Cat #40150-R007) serially diluted against commercial Spike ectodomain (S1+S2) was established to qualify the readout. ELISA measurements for serum samples analyzed are reported as % anti-spike activity which is calculated as the % of the maximal activity of anti-Spike S1 mAb (1/10 dilution) against 5 ng of Spike ectodomain (S1+52). FIG. 8, Panel B, shows serum antibody binding titers to commercial SARS-CoV-2 ectodomain (S1+S2) in sera from bacTRL-Spike-treated mice collected at day 14, 21 and day 40. ELISA titer for anti-Spike immunoreactivity in serum was determined at the dilution where half-maximal percent binding activity was observed (% EC₅₀) and is reported as the inverse dilution at this value (dilution factor at % EC₅₀). P values were derived by unpaired t-test using GraphPad Prism (v8.4.2).

FIG. 8 demonstrates the generation of Spike reactivity in bacTRL-Spike immunized mice. Anti-Spike antibody seroconversion was detected as early as 14 days after oral immunization, with antibody titers against Spike (S1+S2) antigen averaging 132.4. Notably enhanced anti-spike immunoreactivity was observed in samples collected at 21 days after immunization with peak antibody titers averaging 811.42. Anti-spike immunoreactivity persisted in sera samples collected at the end of the study on day 40, at an average titer of 425.7 (FIG. 8 Panel B). No detectable Spike (S1+S2) antigen binding was detected in sera from mice treated with either oral bacTRL-GLuc or oral saline. Accordingly, when expressed by cells of the intestinal lining, bacTRL-Spike derived full-length SARS-CoV-2 Spike protein is able to elicit an antigen-specific systemic humoral response, demonstrating anti-Spike reactivity lasting at least at 40 days after a priming immunization regimen.

Fecal samples were also collected from the bacTRL-Spike treated and control mice on day 14, 21 and 40 to evaluate potential mucosal immunity induced by bacTRL-Spike mediated spike gene delivery to the large intestinal lining. IgA-specific Immunoreactivity of fecal extracts from mice immunized with bacTRL-Spike was measured using the same ELISA against a trimerization-stabilized recombinant SARS-CoV-2 Spike protein construct constituting the S1+S2 ectodomain (Product #46328, LakePharma, CA, USA). FIG. 9, Panel A, shows mean (±SE) % anti-Spike IgA binding activity measured by ELISA against the trimeric Spike ectodomain (S1+S2) using serial titration of fecal extracts derived from bacTRL-Spike-treated and saline mice (day 21 post-immunization). ELISA measurements for fecal extracts analyzed are reported as % anti-spike activity, which is calculated as the % of the maximal activity against 5 ng of Spike ectodomain (S1+S2). FIG. 9, Panel B, shows fecal IgA antibody binding titers to SARS-CoV-2 ectodomain (S1+S2) in extracts from bacTRL-Spike-treated mice collected at day 21. ELISA titer for anti-Spike immunoreactivity in fecal extracts was determined at the dilution where half-maximal percent binding activity was observed (% EC₅₀) and is reported as the inverse dilution at this value (dilution factor at % EC₅₀). P values were derived using two-way ANOVA.

As shown in FIG. 9, IgA-based immunoreactivity against the Spike (S1+S2) ectodomain was significantly elevated in fecal samples collected from bacTRL-Spike-treated mice (Panel A), demonstrating a mean IgA binding titer of 164.5 (Panel B). No detectable spike binding IgA activity was detected in fecal samples from mice treated with saline. These results confirm that antigenic expression of spike transgene in intestinal epithelial cells delivered by bacTRL-Spike is presenting Spike protein complexes in a relevant conformation capable of eliciting mucosal IgA immunity to SARS-CoV-2 spike protein. Oral vaccine delivery confers mucosal immunity within the intestinal tissues. This local mucosal immune protection is known to also spread to other mucosal surfaces as well.

### Generation of Neutralizing Anti-Spike Antibodies.

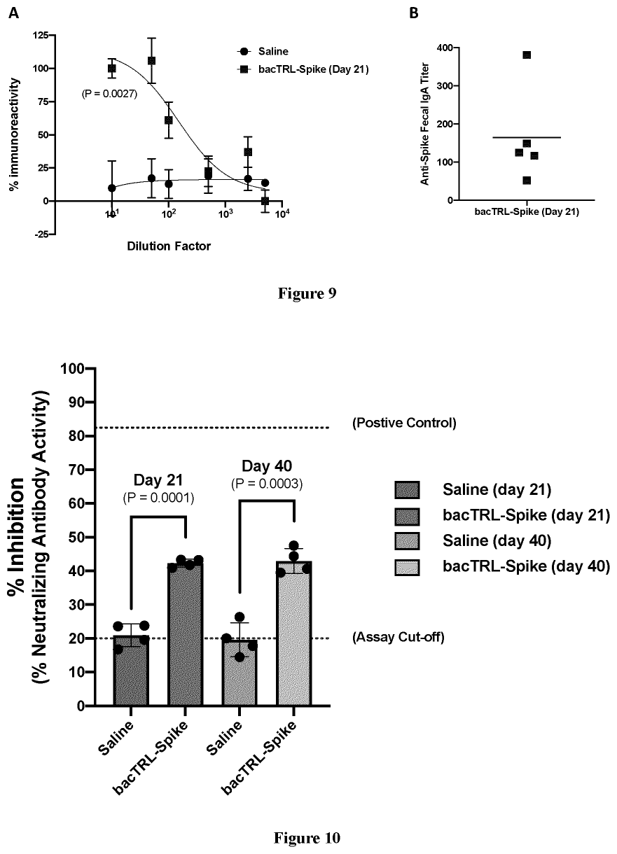

To confirm the ability of orally administered bacTRL-Spike to generate neutralizing antibodies targeting spike, a commercially available ELISA-based assay was used to screen mouse serum samples for inhibition of spike binding to its cognate human receptor, ACE2. Competitive activity of antibodies capable of inhibiting the association of the spike protein's Receptor Binding Motif (RBM) to the human host receptor, ACE2, has been previously established as a surrogate measure of potential neutralizing functionality. Serum samples purified from bacTRL-Spike-treated mice on day 21 and day 40 post-immunization were examined at 1:10 dilution for spike-RBD binding inhibition using a commercially available SARS-CoV-2 neutralizing antibody ELISA assay kit (Creative Diagnostics); the results are shown in FIG. 10. The kit utilizes a soluble HRP-conjugated spike-RBD construct (HRP-RBD) as a molecular target. As a blocking ELISA detection tool, unbound HRP-RBD and HRP-RBD bound by non-neutralizing antibodies are captured by hACE2 and measured using colorimetric HRP substrate reactivity (OD₄₅₀). Reactions with negative and positive control reagents establish assay parameters to determine dynamic range of the readout for appropriate interpretation of percent inhibition, which is presented as rate of inhibition relative to the assay-defined negative control. The positive control reagent in the kit produced a maximum inhibition of 82.5% within these parameters (upper dotted line). Defined assay baseline cut-off is reported to be % inhibition below 20% (lower dotted line). P values were derived using an unpaired t-test.

As shown in FIG. 10, bacTRL-Spike induced anti-spike antibodies effectively competed with ACE2 binding to the spike-RBD domain, with a mean percent inhibition of 42.3% in day 21 sera and 43.0% in day 40 sera relative to the assay negative control. In contrast, serum collected from mice treated with oral saline displayed negligible competitive activity towards inhibiting ACE2 binding, with measures approaching the established cut-off of this commercial ELISA assay. As the primary receptor for SARS-CoV-2 cellular entry, the generation of antibodies that can block the interaction with human ACE2 is generally accepted as important in the context of host protective immunity.

The present invention has been described with regard to one or more embodiments. However, it will be apparent to persons skilled in the art that a number of variations and modifications can be made without departing from the scope of the invention as defined in the claims.

The contents of United States Provisional Patent Application Nos. 63/079,841 (filed Sep. 17, 2020) and 62/981,464 (filed Feb. 25, 2020) are hereby incorporated by reference in their entirety.

1-32. (canceled)

33. A method for delivering a payload nucleic acid into colonic epithelial cells, colonic immune cells, and/or cells of the lamina propria of a subject and causing the cells to produce a payload encoded by the payload nucleic acid,
the method comprising administering to the subject a Bifidobacterium sp. bacterium comprising a plasmid and a transporter nucleic acid such that the bacterium colonizes the colon of the subject;
the transporter nucleic acid is in operative association with a first promoter and a first terminator configured to express the transporter nucleic acid in the bacterium;
the transporter nucleic acid encoding a transporter polypeptide comprising, in an amino-terminal to carboxy-terminal order, a bacterial secretion signal peptide, a DNA-binding domain, and a cell penetrating peptide, the DNA-binding domain configured for association with the plasmid to form a polypeptide-plasmid complex, the bacterial secretion signal peptide configured for secretion of the polypeptide-plasmid complex from the bacterium, and the cell penetrating peptide configured for importing the polypeptide-plasmid complex into a colonic epithelial cell, a colonic immune cell, and/or a cell of the lamina propria of the subject; and
the plasmid comprising a payload nucleic acid having a sequence encoding a payload protein or a payload ribonucleic acid, the payload nucleic acid in operative association with a second promoter and a second terminator configured to express the payload nucleic acid in the colonic epithelial cell, the colonic immune cell, or the cell of the lamina propria and produce the payload protein or the payload ribonucleic acid,
optionally wherein the Bifidobacterium sp. bacterium is Bifidobacterium longum;
optionally wherein the plasmid further comprises the transporter nucleic acid.

34. (canceled)

35. The method of claim 33, wherein the bacterium is administered in a pharmaceutical composition further comprising a pharmaceutically acceptable excipient;
optionally wherein the pharmaceutical composition is orally administered;
optionally wherein the pharmaceutical composition is administered in combination with an immunologic adjuvant;
optionally wherein the bacterium is lyophilized in the pharmaceutical composition;
optionally wherein the pharmaceutical composition is administered to the subject in a dose of 10⁵ to 10¹¹ colony forming units (CFUs), or optionally at a dose of 10⁸ to 10¹⁰ CFUs.

36-39. (canceled)

40. The method of claim 33, wherein the bacterium is a first bacterium and is administered in combination with a second bacterium as defined in claim 33, wherein the payload protein or the payload ribonucleic acid encoded by the payload nucleic acid of the first bacterium is distinct from the payload protein or the payload ribonucleic acid encoded by the payload nucleic acid of the second bacterium;
optionally wherein the first bacterium and the second bacterium are formulated in a single dosage form for co-administration, or
wherein the first bacterium and the second bacterium are formulated as separate dosage forms.

41-42. (canceled)

43. A DNA vaccine comprising:
a Bifidobacterium sp. bacterium comprising a plasmid and a transporter nucleic acid the transporter nucleic acid in operative association with a first promoter and a first terminator configured to express the transporter nucleic acid in the bacterium;
the transporter nucleic acid encoding a transporter polypeptide comprising, in an amino-terminal to carboxy-terminal order, a bacterial secretion signal peptide, a DNA-binding domain, and a cell penetrating peptide, the DNA-binding domain configured for association with the plasmid to form a polypeptide-plasmid complex, the bacterial secretion signal peptide configured for secretion of the polypeptide-plasmid complex from the bacterium, and the cell penetrating peptide configured for importing the polypeptide-plasmid complex into a cell of a subject; and
the plasmid comprising a payload nucleic acid encoding a payload protein, the payload nucleic acid in operative association with a second promoter and a second terminator configured to express the payload gene in the cell and produce the payload protein, wherein the payload protein is a component of a pathogen or wherein the payload protein comprises an antigen that is specific for or associated with a pathology, optionally wherein the pathology is a cancer, optionally wherein the DNA vaccine causes an adaptive immune response in the subject against the pathogen or the pathology following administration of the DNA vaccine to the subject;
optionally wherein the Bifidobacterium sp. bacterium is Bifidobacterium longum;
optionally wherein the plasmid further comprises the transporter nucleic acid.

44-51. (canceled)

52. The DNA vaccine of claim 43, wherein
the pathogen is a virus, a bacteria or a parasite,
optionally wherein the pathogen is a virus,
optionally a coronavirus, optionally a betacoronavirus, optionally SARS-CoV-2.

53-54. (canceled)

55. The DNA vaccine of claim 52, wherein the pathogen is a coronavirus, wherein the payload protein comprises a spike protein or an antigenic fragment thereof, a matrix protein or an antigenic fragment thereof, or a nucleocapsid protein or an antigenic fragment thereof,
optionally wherein the payload protein comprises a spike protein or an antigenic fragment or derivative thereof, a matrix protein or an antigenic fragment or derivative thereof, or a nucleocapsid protein or an antigenic fragment or derivative thereof;
optionally wherein the payload protein comprises a spike protein fragment or a derivative that is at least 80% identical to a wildtype sequence, wherein the spike protein fragment comprises a receptor binding domain (RBD) of the spike protein;
optionally wherein the payload protein comprises the amino acid sequence set out in SEQ ID NO:51 or 55, and the spike protein fragment is a derivative that is at least 80% identical to amino acids 13-685 of SEQ ID NO:46;
optionally wherein the payload protein comprises the amino acid sequence set out in any one of SEQ ID NOs:46 and 53-56;
optionally wherein the payload protein comprises amino acids 13-685 of SEQ ID NO:46 or amino acids 13-682 of SEQ ID NO:53, or optionally comprises amino acids 13-1273 of SEQ ID NO:46 or amino acids 13-1270 of SEQ ID NO:53.

56-83. (canceled)

84. A method of vaccinating a subject against a pathogen, the method comprising administering to the subject the DNA vaccine of claim 43, wherein the payload nucleic acid encodes one or more components of the pathogen.

85. A method of vaccinating a subject against a coronavirus, the method comprising administering to the subject the DNA vaccine of claim 52,
optionally wherein the coronavirus is a betacoronavirus, optionally SARS-CoV-2.

86. (canceled)

87. A method of vaccinating a subject against a pathology, the method comprising administering to the subject the DNA vaccine of claim 43, wherein the payload nucleic acid encodes an antigen that is specific for or associated with the pathology, optionally wherein the pathology is a cancer,
wherein the bacterium is administered in a pharmaceutical composition further comprising a pharmaceutically acceptable excipient;
optionally wherein the pharmaceutical composition is orally administered.

88-89. (canceled)

90. The method of claim 87, wherein the pharmaceutical composition is administered in combination with an immunologic adjuvant;
optionally wherein the bacterium is lyophilized in the pharmaceutical composition;
optionally wherein the pharmaceutical composition is administered to the subject in a dose of 10⁵ to 10¹¹ colony forming units (CFUs), or optionally at a dose of 10⁸ to 10¹⁰ CFUs.

91-92. (canceled)

93. The method of claim 84, wherein the bacterium is a first bacterium and is administered in combination with a second bacterium as defined in claim 43, wherein the payload protein encoded by the payload nucleic acid of the first bacterium is distinct from the payload protein or the payload ribonucleic acid encoded by the payload nucleic acid of the second bacterium,
optionally wherein the first bacterium and the second bacterium are formulated in a single dosage form for co-administration;
optionally wherein the first bacterium and the second bacterium are formulated as separate dosage forms;
optionally wherein the payload protein or the payload ribonucleic acid encoded by the payload nucleic acid of the second bacterium comprises one or more immunomodulatory proteins, optionally wherein the one or more immunomodulatory proteins is one or a combination of IL-12, INFg, TNFa, IL-10, IL-8, IL-2, IL-4, Il-15, IL-18, IL1a/b, IL-6, IL-17, CXCL10, CXCL-13, GSMCF, LTa/b, and/or a functional derivative of the foregoing.

94-96. (canceled)

97. The method of claim 33, wherein:
the bacterial secretion signal peptide is an alpha-arabinosidase secretion signal peptide, optionally having SEQ ID NO:13; and/or
the DNA-binding domain has sequence SEQ ID NO: 7; and/or
the cell penetrating peptide has sequence SEQ ID NO: 18;
optionally wherein the transporter polypeptide has sequence SEQ ID NO: 2.

98. The method of claim 33, wherein the payload nucleic acid comprises a basolateral sorting signal for targeting a payload protein to the basolateral cell membrane of the colonic epithelial cell, or wherein the payload nucleic acid comprises an apical sorting signal for targeting a payload protein to the lumenal cell membrane of the colonic epithelial cell.

99. The method of claim 33, wherein the payload protein is a membrane or membrane-associated protein comprising an extracellular domain,
optionally wherein the membrane or membrane-associated protein is an integral membrane protein.

100. The method of claim 33, wherein:
the plasmid further encodes a lipid anchor signal peptide in operative association with the payload nucleic acid to produce the payload protein as a lipid anchored protein; or
the plasmid further encodes a secretion signal peptide in operative association with the payload nucleic acid to secrete the payload protein; or
the plasmid is configured to produce the payload protein as an intracellular protein.

101. The method of claim 33, wherein the payload nucleic acid encodes, alone or in combination with other nucleic acid(s), an antigen from a pathogen, an antigen that is specific for or associated with a pathology, optionally cancer, an immunomodulatory protein, an antibody or antibody fragment or derivative, an enzyme, a receptor, or a therapeutic protein;
optionally wherein the payload protein comprises: a coronavirus spike protein, matrix protein or nucleocapsid protein; or a betacoronavirus spike protein, matrix protein or nucleocapsid protein, optionally wherein the coronavirus spike protein, matrix protein or nucleocapsid protein is SARS-CoV-2 spike protein, matrix protein or nucleocapsid protein;
optionally wherein the payload nucleic acid encodes a plurality of payloads, the plurality of payloads comprising a combination of antigenic proteins from the pathogen.

102. The method of claim 33, wherein the payload nucleic acid encodes a plurality of payloads;
optionally wherein the plurality of payloads comprises one or more immunomodulatory proteins, optionally wherein the one or more immunomodulatory proteins is one or a combination of IL-12, INFg, TNFa, IL-10, IL-8, IL-2, TL-4, 11-15, IL-18, IL1a/b, IL-6, IL-17, CXCL10, CXCL-13, GSMCF, LTa/b, and/or a functional derivative of the foregoing;
optionally wherein the payload nucleic acid comprises a plurality of payload coding sequences, wherein the payload nucleic acid is operatively associated with a single promoter and terminator for expression in the colonic epithelial cells, the colonic immune cells, and/or the cells of the lamina propria, and wherein each payload coding sequence is separated by an IRES element;
or optionally wherein the payload nucleic acid comprises a plurality of payload coding sequences and each payload coding sequence is operatively associated with a separate promoter and terminator for expression in the colonic epithelial cells, the colonic immune cells, and/or the cells of the lamina propria.

103. The method of claim 87, wherein the bacterium is a first bacterium and is administered in combination with a second bacterium as defined in claim 43, wherein the payload protein encoded by the payload nucleic acid of the first bacterium is distinct from the payload protein or the payload ribonucleic acid encoded by the payload nucleic acid of the second bacterium;
optionally wherein the first bacterium and the second bacterium are formulated in a single dosage form for co-administration.
optionally wherein the first bacterium and the second bacterium are formulated as separate dosage forms;
optionally wherein the payload protein or the payload ribonucleic acid encoded by the payload nucleic acid of the second bacterium comprises one or more immunomodulatory proteins, optionally wherein the one or more immunomodulatory proteins is one or a combination of IL-12, INFg, TNFa, IL-10, IL-8, IL-2, IL-4, Il-15, IL-18, IL1a/b, IL-6, IL-17, CXCL10, CXCL-13, GSMCF, LTa/b, and/or a functional derivative of the foregoing.

104. The method of claim 84, wherein:
the bacterial secretion signal peptide is an alpha-arabinosidase secretion signal peptide, optionally having SEQ ID NO:13; and/or
the DNA-binding domain has sequence SEQ ID NO: 7; and/or
the cell penetrating peptide has sequence SEQ ID NO: 18;
optionally wherein the transporter polypeptide has sequence SEQ ID NO: 2.

105. The method of claim 87, wherein:
the bacterial secretion signal peptide is an alpha-arabinosidase secretion signal peptide, optionally having SEQ ID NO:13; and/or
the DNA-binding domain has sequence SEQ ID NO: 7; and/or
the cell penetrating peptide has sequence SEQ ID NO: 18;
optionally wherein the transporter polypeptide has sequence SEQ ID NO: 2.


SEQUENCE LISTING (56 sequences)

SEQ ID NO: 1 - skipped

SEQ ID NO: 2 - protein, 133 aa, Artificial Sequence
  Transporter polypeptide HTP
MTLTGTLRKAFATTLAAAMLIGTLAGCSSAAYNKSDLVSKIAQKSNLTKAQAEAAVNAFQDVFVEAMKSGEGLKLTGLFSAERVKRPARTGRNPRTGEQIDIPASYGVRISAGSLLKKAVTEYGRKKRRQRRR

SEQ ID NO: 3 - protein, 577 aa, Artificial Sequence
  M-IL-12
MCHQQLVISWFSLVFLASPLVAIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSGGGGGGSRNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNASGGGGGGSGGGGGGSIIIGPLIFVFLFSVVIGSIYLFLRKRQPDGP

SEQ ID NO: 4 - DNA, 429 bp, Bifidobacterium longum
gaaaacaatttggagaacctgaccattggcgttttcgccaggacggccggggtcaatgtggagaccatccggttctatcagcgcaagggcttgctcccggaaccggacaagccttacggcagcattcgccgctatggcgagacggatgtaacgcgggtgcgcttcgtgaaatcagcccagcggttgggcttcagcctggatgagatcgccgagctgctgcggctggaggatggcacccattgcgaggaagccagcagcctggccgagcacaagctcaaggacgtgcgcgagaggatggctgacctggcgcgcatggaggccgtgctgtctgatttggtgtgcgcctgccatgcgcgaagggggaacgtttcctgcccgctgatcgcgtcactacagggtggagcaagcttggcaggttcggctatgcct

SEQ ID NO: 5 - protein, 143 aa, Bifidobacterium longum
ENNLENLTIGVFARTAGVNVETIRFYQRKGLLPEPDKPYGSIRRYGETDVTRVRFVKSAQRLGFSLDEIAELLRLEDGTHCEEASSLAEHKLKDVRERMADLARMEAVLSDLVCACHARRGNVSCPLIASLQGGASLAGSAMP

SEQ ID NO: 6 - DNA, 276 bp, Bifidobacterium longum
gcatacaacaagtctgacctcgtttcgaagatcgcccagaagtccaacctgaccaaggctcaggccgaggctgctgttaacgccttccaggatgtgttcgtcgaggctatgaagtccggcgaaggcctgaagctcaccggcctgttctccgctgagcgcgtcaagcgcccggctcgcaccggccgcaacccgcgcactggcgagcagattgacattccggcttcctacggcgttcgtatctccgctggctccctgctgaagaaggccgtcaccgag

SEQ ID NO: 7 - protein, 92 aa, Bifidobacterium longum
AYNKSDLVSKIAQKSNLTKAQAEAAVNAFQDVFVEAMKSGEGLKLTGLFSAERVKRPARTGRNPRTGEQIDIPASYGVRISAGSLLKKAVTE

SEQ ID NO: 8 - DNA, 294 bp, Artificial Sequence
  Zinc finger DNA binding domain
gaaaaactgcgcaacggcagcggcgatccgggcaaaaaaaaacagcatgcgtgcccggaatgcggcaaaagctttagccagagcagcgatctgcagcgccatcagcgcacccataccggcgaaaaaccgtataaatgcccggaatgcggcaaaagctttagccgcagcgatgaactgcagcgccatcagcgcacccataccggcgaaaaaccgtataaatgcccggaatgcggcaaaagctttagccgcagcgatcatctgagccgccatcagcgcacccatcagaacaaaaaa

SEQ ID NO: 9 - protein, 98 aa, Artificial Sequence
  Zinc finger DNA binding domain
EKLRNGSGDPGKKKQHACPECGKSFSQSSDLQRHQRTHTGEKPYKCPECGKSFSRSDELQRHQRTHTGEKPYKCPECGKSFSRSDHLSRHQRTHQNKK

SEQ ID NO: 10 - DNA, 84 bp, Bifidobacterium longum
aagcttgtcgacctgcagccttggcgcttgactccgtacatgagtacggaagtaaggttacgctatccttggctgcagaagctt

SEQ ID NO: 11 - DNA, 41 bp, Artificial Sequence
  Zinc finger DNA binding domain recognition site
aagcttttttttttttggggcggctttttttttttaagctt

SEQ ID NO: 12 - DNA, 90 bp, Bifidobacterium longum
atgaccctgaccggcaccctgcgcaaggccttcgccaccaccctggccgccgccatgctgatcggcaccctggccggctgctcctccgcc

SEQ ID NO: 13 - protein, 30 aa, Bifidobacterium longum
MTLTGTLRKAFATTLAAAMLIGTLAGCSSA

SEQ ID NO: 14 - DNA, 111 bp, Bifidobacterium longum
atgaaacatcggaaacccgcaccggcctggcataggctggggctgaagattagcaagaaagtggtggtcggcatcaccgccgcggcgaccgccttcggcggactggcaatc

SEQ ID NO: 15 - protein, 37 aa, Bifidobacterium longum
MKHRKPAPAWHRLGLKISKKVVVGITAAATAFGGLAI

SEQ ID NO: 16 - DNA, 138 bp, Bifidobacterium longum
atgaaacatcggaaacccgcaccggcctggcataggctggggctgaagattagcaagaaagtggtggtcggcatcaccgccgcggcgaccgccttcggcggactggcaatcgccagcaccgcagcacaggccagcacc

SEQ ID NO: 17 - protein, 46 aa, Bifidobacterium longum
MKHRKPAPAWHRLGLKISKKVVVGITAAATAFGGLAIASTAAQAST

SEQ ID NO: 18 - protein, 11 aa, Human Immunodeficiency virus
YGRKKRRQRRR

SEQ ID NO: 19 - protein, 16 aa, Antennapedia Drosophilamelanogaster
RQIKIWFQNRRMKWKK

SEQ ID NO: 20 - protein, 17 aa, Human immunodeficiency virus
TRQARRNRRRRWRERQF

SEQ ID NO: 21 - protein, 17 aa, Human immunodeficiency virus
NAKTRRHERRRKLAIER

SEQ ID NO: 22 - protein, 27 aa, Simian Virus 40
GALFLGFLGAAGSTMGAWSQPKKKRKV

SEQ ID NO: 23 - protein, 27 aa, Artificial Sequence
  Transportan (Galanin-mastoparan) CPP
GWTLNSAGYLLGKINLKALAALAKKIL

SEQ ID NO: 24 - protein, 21 aa, Simian Virus
  MISC_FEATURE (20)..(20): X may be Arginine or Lysine
KETWWETWWTEWSQPKKKRXV

SEQ ID NO: 25 - protein, 10 aa, Human immunodeficiency virus 1
GRKKRRQRRR

SEQ ID NO: 26 - protein, 10 aa, Artificial Sequence
  HRSV CPP
RRIPNRRPRR

SEQ ID NO: 27 - protein, 5 aa, Artificial Sequence
  AIP6 CPP
RLRWR

SEQ ID NO: 28 - protein, 22 aa, Artificial Sequence
  ARF(1-22) CPP
MVRRFLVTLRIRRACGPPRVRV

SEQ ID NO: 29 - protein, 18 aa, Artificial Sequence
  pVEC CPP
LLIILRRRIRKQAHAHSK

SEQ ID NO: 30 - protein, 18 aa, Artificial Sequence
  MAP17 CPP
QLALQLALQALQAALQLA

SEQ ID NO: 31 - protein, 26 aa, Artificial Sequence
  VT5 CPP
DPKGDPKGVTVTVTVTVTGKGDPKPD

SEQ ID NO: 32 - protein, 24 aa, Artificial Sequence
  Bac7 CPP
RRIRPRPPRLPRPRPRPLPFPRPG

SEQ ID NO: 33 - protein, 9 aa, Artificial Sequence
  (PPR)n CPP
PPRPPRPPR

SEQ ID NO: 34 - protein, 20 aa, Herpes Simplex virus 1
HGLASTLTRWAHYNALIRAF

SEQ ID NO: 35 - protein, 30 aa, Artificial Sequence
  GALA CPP
WEAALAEALAEALAEHLAEALAEALEALAA

SEQ ID NO: 36 - protein, 24 aa, Influenza
GLFEAIEGFIENGWEGMIDGWYGC

SEQ ID NO: 37 - protein, 17 aa, Artificial Sequence
  C105Y CPP
CSIPPEVKFNKPFVYLI

SEQ ID NO: 38 - protein, 6 aa, Artificial Sequence
  PFVYLI CPP
PFVYLI

SEQ ID NO: 39 - protein, 15 aa, Artificial Sequence
  Pep-7 CPP
SDLWEMMMVSLACQY

SEQ ID NO: 40 - protein, 20 aa, Artificial Sequence
  CADY CPP
GLWRALWRLLRSLWRLLWRA

SEQ ID NO: 41 - protein, 18 aa, Artificial Sequence
  MAP CPP
KLALKLALKALKAALKLA

SEQ ID NO: 42 - protein, 9 aa, Artificial Sequence
  R6W3 CPP
RRWWRRWRR

SEQ ID NO: 43 - DNA, 5714 bp, Artificial Sequence
  pFRG3.5-CMV-GLuc
atgtgagcaaaaggccagcaaaaggccagggaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacgggtttttttggggcggctttgaattcttttttttggggcggctttttttttatgcgctcacgcaactggtccagaaccttgaccgaacgcagcggtggtaacggcgcagtggcggttttcatggcttgttatgactgtttttttggggtacagtctatgcctcgggcatccaagcagcaagcgcgttacgccgtgggtcgatgtttgatgttatggagcagcaacgatgttacgcagcagggcagtcgccctaaaacaaagttaaacatcatgagggaagcggtgatcgccgaagtatcgactcaactatcagaggtagttggcgtcatcgagcgccatctcgaaccgacgttgctggccgtacatttgtacggctccgcagtggatggcggcctgaagccacacagtgatattgatttgctggttacggtgaccgtaaggcttgatgaaacaacgcggcgagctttgatcaacgaccttttggaaacttcggcttcccctggagagagcgagattctccgcgctgtagaagtcaccattgttgtgcacgacgacatcattccgtggcgttatccagctaagcgcgaactgcaatttggagaatggcagcgcaatgacattcttgcaggtatcttcgagccagccacgatcgacattgatctggctatcttgctgacaaaagcaagagaacatagcgttgccttggtaggtccagcggcggaggaactctttgatccggttcctgaacaggatctatttgaggcgctaaatgaaaccttaacgctatggaactcgccgcccgactgggctggcgatgagcgaaatgtagtgcttacgttgtcccgcatttggtacagcgcagtaaccggcaaaatcgcgccgaaggatgtcgctgccgactgggcaatggagcgcctgccggcccagtatcagcccgtcatacttgaagctagacaggcttatcttggacaagaagaagatcgcttggcctcgcgcgcagatcagttggaagaatttgtccactacgtgaaaggcgagatcaccaaggtagtcggcaaataaggtaccgttaggcgttttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccggactctagaggatcgaagctagccaccatgggagtcaaagttctgtttgccctgatctgcatcgctgtggccgaggccaagcccaccgagaacaacgaagacttcaacatcgtggccgtggccagcaacttcgcgaccacggatctcgatgctgaccgcgggaagttgcccggcaagaagctgccgctggaggtgctcaaagagatggaagccaatgcccggaaagctggctgcaccaggggctgtctgatctgcctgtcccacatcaagtgcacgcccaagatgaagaagttcatcccaggacgctgccacacctacgaaggcgacaaagagtccgcacagggcggcataggcgaggcgatcgtcgacattcctgagattcctgggttcaaggacttggagcccatggagcagttcatcgcacaggtcgatctgtgtgtggactgcacaactggctgcctcaaagggcttgccaacgtgcagtgttctgacctgctcaagaagtggctgccgcaacgctgtgcgacctttgccagcaagatccagggccaggtggacaagatcaagggggccggtggtgactaatcgatccctaccggttagtaatgagtttaaacgggggaggctaactgaaacacggaaggagacaataccggaaggaacccgcgctatgacggcaataaaaagacagaataaaacgcacgggtgttgggtcgtttgttcataaacgcggggttcggtcccagggctggcactctgtcgataccccaccgagaccccattggggccaatacgcccgcgtttcttccttttccccaccccaccccccaagttcgggtgaaggcccagggctcgcagccaacgtcggggcggcaggccctgccatagctttttggggcggcttttctcgagtccttccttaaggacgtgctcgtcaatttttgttttgagagtcatctattcggatgcttttcatgaagttttttatgaccctgaccggcaccctgcgcaaggccttcgccaccaccctggccgccgccatgctgatcggcaccctggccggctgctcctccgccgcatacaacaagtctgacctcgtttcgaagatcgcccagaagtccaacctgaccaaggctcaggccgaggctgctgttaacgccttccaggatgtgttcgtcgaggctatgaagtccggcgaaggcctgaagctcaccggcctgttctccgctgagcgcgtcaagcgcccggctcgcaccggccgcaacccgcgcactggcgagcagattgacattccggcttcctacggcgttcgtatctccgctggctccctgctgaagaaggccgtcaccgagtatggacggaagaagcgcaggcagcgacggcgatgagggtttgcgcttgcgtcgtggagggagcggaacgccgaaaaaggatccacgcgctggagatgttcaacgagtagatcgccacggcgacctccttccacgcgtgcgggcacggggattctcaaggggccggcccgaggccccttgagcccgccgggaggcgcccccggcagggcgggaatccaaagggcggagccctgtggccctccccgggcaggggcgggatcgtcaagggcggagcccttggccccctcgggagagcgcactgacacaatgctacctccggtagcattaagtgcgccctccgccatgcggagggacgggccgcgaccggatatgcggggaacgtccacgacgcgtcttccgtgtcctccgtcctgccttgtgccgtcgattataatcctcggataacggacgatgattgaaccgattggaggaaacgagatgccgaagagtttcgcgcagcagatcgaggacgacgagaacaagatcaagcgcatccgggagcaccagcgcatggtcagggccaaacaagccaaacaggagcgcaacgcccgcaccaaaaggctcgtggagaccggagcgatagtggaaaaagcgcacggcggcgcgtacgacgacgaaggacggcagacgttctcggatggtctgaacggcatcatctcggtctacgacccgtcccgcggcggcaacgtggacatgagagtcatcgacgtaatcgaccggcgcatccccagattgccaaggtccgaaaccacaacaggcacggcagcggcggcatcgcgaaccgtgcaagccaccgcaccacagccagcccacgcccaaccgcaaagcttcacgcccaacccccagcgcatcgaacaccggacaggagcacagcagccggatcggtgggcctagccgggacggcgcagccctcgctccgctgggctgaaatatctgacttggttcgtaacatatttccgacatgtcggaaatatgtcgtatcatcgttgctatgagtactgaacttcgtgagcagtgggagcagctttacctgccgctgcgcccgctgtgcacgaacgacttcatcgagggcgtgtaccggcagcccagggcgaaggcgctggagggctaccggtacatcgaggcgaaccccaagaccgtcagcgacctgctcgtggtcgacatcgacgacgcgaacgcccgcgcgatggccctatgggagcatgaggggatgctgcccaacgtgatcgtggagaacccgagaaacggccacgcgcacgccgtgtgggcgctggccgccccgttcagtcgcaccgaatacgcgcggcgcaagccgctcgccctggccgcggcggtcacggaggggctgcgccgctcgtgcgacggggacaagggatattcggggctgatgaccaagaacccgctgcacgaggcgtggaacagcgaactggtcaccgatcacctgtacgggctggacgaactgcgcgaggcgctggaggagtccggggacatgccgccggcctcgtggaagcgcacgaaacggcgcaacgtggtcggattgggccgcaactgccatctgttcgagacggcgcgcacatgggcgtacagggaggtgcgccaccactggggagacagcgaggggctgcggctggccatctcggccgaggcgcacgaaatgaacgccgaactgttccccgagccactggcatggtcggaggtcgagcagatcgccaagagcatccacaagtggatcgtcacccaaagccgcatgtggcgcgacggccccaccgtctacgaggcgacattcatcaccgtccaaagcgcccgagggaaaaagtcaggcaaggccagacgagaaacattcgaactattcgcgagcgaggagatgacgtaa

SEQ ID NO: 44 - DNA, 8978 bp, Artificial Sequence
  pFRG3.5-CMV-SGene
atgtgagcaaaaggccagcaaaaggccagggaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacgggtttttttggggcggctttgaattcttttttttggggcggctttttttttatgcgctcacgcaactggtccagaaccttgaccgaacgcagcggtggtaacggcgcagtggcggttttcatggcttgttatgactgtttttttggggtacagtctatgcctcgggcatccaagcagcaagcgcgttacgccgtgggtcgatgtttgatgttatggagcagcaacgatgttacgcagcagggcagtcgccctaaaacaaagttaaacatcatgagggaagcggtgatcgccgaagtatcgactcaactatcagaggtagttggcgtcatcgagcgccatctcgaaccgacgttgctggccgtacatttgtacggctccgcagtggatggcggcctgaagccacacagtgatattgatttgctggttacggtgaccgtaaggcttgatgaaacaacgcggcgagctttgatcaacgaccttttggaaacttcggcttcccctggagagagcgagattctccgcgctgtagaagtcaccattgttgtgcacgacgacatcattccgtggcgttatccagctaagcgcgaactgcaatttggagaatggcagcgcaatgacattcttgcaggtatcttcgagccagccacgatcgacattgatctggctatcttgctgacaaaagcaagagaacatagcgttgccttggtaggtccagcggcggaggaactctttgatccggttcctgaacaggatctatttgaggcgctaaatgaaaccttaacgctatggaactcgccgcccgactgggctggcgatgagcgaaatgtagtgcttacgttgtcccgcatttggtacagcgcagtaaccggcaaaatcgcgccgaaggatgtcgctgccgactgggcaatggagcgcctgccggcccagtatcagcccgtcatacttgaagctagacaggcttatcttggacaagaagaagatcgcttggcctcgcgcgcagatcagttggaagaatttgtccactacgtgaaaggcgagatcaccaaggtagtcggcaaataaggtaccgttaggcgttttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccggactctagaggatcgaagctagccaccatgtttgtttttcttgttttattgccactagtctctagtcagtgtgttaatcttacaaccagaactcaattaccccctgcatacactaattctttcacacgtggtgtttattaccctgacaaagttttcagatcctcagttttacattcaactcaggacttgttcttacctttcttttccaatgttacttggttccatgctatacatgtctctgggaccaatggtactaagaggtttgataaccctgtcctaccatttaatgatggtgtttattttgcttccactgagaagtctaacataataagaggctggatttttggtactactttagattcgaagacccagtccctacttattgttaataacgctactaatgttgttattaaagtctgtgaatttcaattttgtaatgatccatttttgggtgtttattaccacaaaaacaacaaaagttggatggaaagtgagttcagagtttattctagtgcgaataattgcacttttgaatatgtctctcagccttttcttatggaccttgaaggaaaacagggtaatttcaaaaatcttagggaatttgtgtttaagaatattgatggttattttaaaatatattctaagcacacgcctattaatttagtgcgtgatctccctcagggtttttcggctttagaaccattggtagatttgccaataggtattaacatcactaggtttcaaactttacttgctttacatagaagttatttgactcctggtgattcttcttcaggttggacagctggtgctgcagcttattatgtgggttatcttcaacctaggacttttctattaaaatataatgaaaatggaaccattacagatgctgtagactgtgcacttgaccctctctcagaaacaaagtgtacgttgaaatccttcactgtagaaaaaggaatctatcaaacttctaactttagagtccaaccaacagaatctattgttagatttcctaatattacaaacttgtgcccttttggtgaagtttttaacgccaccagatttgcatctgtttatgcttggaacaggaagagaatcagcaactgtgttgctgattattctgtcctatataattccgcatcattttccacttttaagtgttatggagtgtctcctactaaattaaatgatctctgctttactaatgtctatgcagattcatttgtaattagaggtgatgaagtcagacaaatcgctccagggcaaactggaaagattgctgattataattataaattaccagatgattttacaggctgcgttatagcttggaattctaacaatcttgattctaaggttggtggtaattataattacctgtatagattgtttaggaagtctaatctcaaaccttttgagagagatatttcaactgaaatctatcaggccggtagcacaccttgtaatggtgttgaaggttttaattgttactttcctttacaatcatatggtttccaacccactaatggtgttggttaccaaccatacagagtagtagtactttcttttgaacttctacatgcaccagcaactgtttgtggacctaaaaagtctactaatttggttaaaaacaaatgtgtcaatttcaacttcaatggtttaacaggcacaggtgttcttactgagtctaacaaaaagtttctgcctttccaacaatttggcagagacattgctgacactactgatgctgtccgtgatccacagacacttgagattcttgacattacaccatgttcttttggtggtgtcagtgttataacaccaggaacaaatacttctaaccaggttgctgttctttatcaggatgttaactgcacagaagtccctgttgctattcatgcagatcaacttactcctacttggcgtgtttattctacaggttctaatgtttttcaaacacgtgcaggctgtttaataggggctgaacatgtcaacaactcatatgagtgtgacatacccattggtgcaggtatatgcgctagttatcagactcagactaattctcctcggcgggcacgtagtgtagctagtcaatccatcattgcctacactatgtcacttggtgcagaaaattcagttgcttactctaataactctattgccatacccacaaattttactattagtgttaccacagaaattctaccagtgtctatgaccaagacatcagtagattgtacaatgtacatttgtggtgattcaactgaatgcagcaatcttttgttgcaatatggcagtttttgtacacaattaaaccgtgctttaactggaatagctgttgaacaagacaaaaacacccaagaagtttttgcacaagtcaaacaaatttacaaaacaccaccaattaaagattttggtggttttaatttttcacaaatattaccagatccatcaaaaccaagcaagaggtcatttattgaagatctacttttcaacaaagtgacacttgcagatgctggcttcatcaaacaatatggtgattgccttggtgatattgctgctagagacctcatttgtgcacaaaagtttaacggccttactgttttgccacctttgctcacagatgaaatgattgctcaatacacttctgcactgttagcgggtacaatcacttctggttggacctttggtgcaggtgctgcattacaaataccatttgctatgcaaatggcttataggtttaatggtattggagttacacagaatgttctctatgagaaccaaaaattgattgccaaccaatttaatagtgctattggcaaaattcaagactcactttcttccacagcaagtgcacttggaaaacttcaagatgtggtcaaccaaaatgcacaagctttaaacacgcttgttaaacaacttagctccaattttggtgcaatttcaagtgttttaaatgatatcctttcacgtcttgacaaagttgaggctgaagtgcaaattgataggttgatcacaggcagacttcaaagtttgcagacatatgtgactcaacaattaattagagctgcagaaatcagagcttctgctaatcttgctgctactaaaatgtcagagtgtgtacttggacaatcaaaaagagttgatttttgtggaaagggctatcatcttatgtccttccctcagtcagcacctcatggtgtagtcttcttgcatgtgacttatgtccctgcacaagaaaagaacttcacaactgctcctgccatttgtcatgatggaaaagcacactttcctcgtgaaggtgtctttgtttcaaatggcacacactggtttgtaacacaaaggaatttttatgaaccacaaatcattactacagacaacacatttgtgtctggtaactgtgatgttgtaataggaattgtcaacaacacagtttatgatcctttgcaacctgaattagactcattcaaggaggagttagataaatattttaagaatcatacatcaccagatgttgatttaggtgacatctctggcattaatgcttcagttgtaaacattcaaaaagaaattgaccgcctcaatgaggttgccaagaatttaaatgaatctctcatcgatctccaagaacttggaaagtatgagcagtatataaaatggccatggtacatttggctaggttttatagctggcttgattgccatagtaatggtgacaattatgctttgctgtatgaccagttgctgtagttgtctcaagggctgttgttcttgtggatcctgctgcaaatttgatgaagacgactctgagccagtgctcaaaggagtcaaattacattacacataatcgatccctaccggttagtaatgagtttaaacgggggaggctaactgaaacacggaaggagacaataccggaaggaacccgcgctatgacggcaataaaaagacagaataaaacgcacgggtgttgggtcgtttgttcataaacgcggggttcggtcccagggctggcactctgtcgataccccaccgagaccccattggggccaatacgcccgcgtttcttccttttccccaccccaccccccaagttcgggtgaaggcccagggctcgcagccaacgtcggggcggcaggccctgccatagctttttggggcggcttttctcgagtccttccttaaggacgtgctcgtcaatttttgttttgagagtcatctattcggatgcttttcatgaagttttttatgaccctgaccggcaccctgcgcaaggccttcgccaccaccctggccgccgccatgctgatcggcaccctggccggctgctcctccgccgcatacaacaagtctgacctcgtttcgaagatcgcccagaagtccaacctgaccaaggctcaggccgaggctgctgttaacgccttccaggatgtgttcgtcgaggctatgaagtccggcgaaggcctgaagctcaccggcctgttctccgctgagcgcgtcaagcgcccggctcgcaccggccgcaacccgcgcactggcgagcagattgacattccggcttcctacggcgttcgtatctccgctggctccctgctgaagaaggccgtcaccgagtatggacggaagaagcgcaggcagcgacggcgatgagggtttgcgcttgcgtcgtggagggagcggaacgccgaaaaaggatccacgcgctggagatgttcaacgagtagatcgccacggcgacctccttccacgcgtgcgggcacggggattctcaaggggccggcccgaggccccttgagcccgccgggaggcgcccccggcagggcgggaatccaaagggcggagccctgtggccctccccgggcaggggcgggatcgtcaagggcggagcccttggccccctcgggagagcgcactgacacaatgctacctccggtagcattaagtgcgccctccgccatgcggagggacgggccgcgaccggatatgcggggaacgtccacgacgcgtcttccgtgtcctccgtcctgccttgtgccgtcgattataatcctcggataacggacgatgattgaaccgattggaggaaacgagatgccgaagagtttcgcgcagcagatcgaggacgacgagaacaagatcaagcgcatccgggagcaccagcgcatggtcagggccaaacaagccaaacaggagcgcaacgcccgcaccaaaaggctcgtggagaccggagcgatagtggaaaaagcgcacggcggcgcgtacgacgacgaaggacggcagacgttctcggatggtctgaacggcatcatctcggtctacgacccgtcccgcggcggcaacgtggacatgagagtcatcgacgtaatcgaccggcgcatccccagattgccaaggtccgaaaccacaacaggcacggcagcggcggcatcgcgaaccgtgcaagccaccgcaccacagccagcccacgcccaaccgcaaagcttcacgcccaacccccagcgcatcgaacaccggacaggagcacagcagccggatcggtgggcctagccgggacggcgcagccctcgctccgctgggctgaaatatctgacttggttcgtaacatatttccgacatgtcggaaatatgtcgtatcatcgttgctatgagtactgaacttcgtgagcagtgggagcagctttacctgccgctgcgcccgctgtgcacgaacgacttcatcgagggcgtgtaccggcagcccagggcgaaggcgctggagggctaccggtacatcgaggcgaaccccaagaccgtcagcgacctgctcgtggtcgacatcgacgacgcgaacgcccgcgcgatggccctatgggagcatgaggggatgctgcccaacgtgatcgtggagaacccgagaaacggccacgcgcacgccgtgtgggcgctggccgccccgttcagtcgcaccgaatacgcgcggcgcaagccgctcgccctggccgcggcggtcacggaggggctgcgccgctcgtgcgacggggacaagggatattcggggctgatgaccaagaacccgctgcacgaggcgtggaacagcgaactggtcaccgatcacctgtacgggctggacgaactgcgcgaggcgctggaggagtccggggacatgccgccggcctcgtggaagcgcacgaaacggcgcaacgtggtcggattgggccgcaactgccatctgttcgagacggcgcgcacatgggcgtacagggaggtgcgccaccactggggagacagcgaggggctgcggctggccatctcggccgaggcgcacgaaatgaacgccgaactgttccccgagccactggcatggtcggaggtcgagcagatcgccaagagcatccacaagtggatcgtcacccaaagccgcatgtggcgcgacggccccaccgtctacgaggcgacattcatcaccgtccaaagcgcccgagggaaaaagtcaggcaaggccagacgagaaacattcgaactattcgcgagcgaggagatgacgtaa

SEQ ID NO: 45 - DNA, 12065 bp, Artificial Sequence
  pFRG3.5-CMV-COVID19-SiMiN
atgtgagcaaaaggccagcaaaaggccagggaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacgggtttttttggggcggctttgaattcttttttttggggcggctttttttttatgcgctcacgcaactggtccagaaccttgaccgaacgcagcggtggtaacggcgcagtggcggttttcatggcttgttatgactgtttttttggggtacagtctatgcctcgggcatccaagcagcaagcgcgttacgccgtgggtcgatgtttgatgttatggagcagcaacgatgttacgcagcagggcagtcgccctaaaacaaagttaaacatcatgagggaagcggtgatcgccgaagtatcgactcaactatcagaggtagttggcgtcatcgagcgccatctcgaaccgacgttgctggccgtacatttgtacggctccgcagtggatggcggcctgaagccacacagtgatattgatttgctggttacggtgaccgtaaggcttgatgaaacaacgcggcgagctttgatcaacgaccttttggaaacttcggcttcccctggagagagcgagattctccgcgctgtagaagtcaccattgttgtgcacgacgacatcattccgtggcgttatccagctaagcgcgaactgcaatttggagaatggcagcgcaatgacattcttgcaggtatcttcgagccagccacgatcgacattgatctggctatcttgctgacaaaagcaagagaacatagcgttgccttggtaggtccagcggcggaggaactctttgatccggttcctgaacaggatctatttgaggcgctaaatgaaaccttaacgctatggaactcgccgcccgactgggctggcgatgagcgaaatgtagtgcttacgttgtcccgcatttggtacagcgcagtaaccggcaaaatcgcgccgaaggatgtcgctgccgactgggcaatggagcgcctgccggcccagtatcagcccgtcatacttgaagctagacaggcttatcttggacaagaagaagatcgcttggcctcgcgcgcagatcagttggaagaatttgtccactacgtgaaaggcgagatcaccaaggtagtcggcaaataaggtaccgttaggcgttttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccggactctagaggatcgaagctagccaccatgtttgtttttcttgttttattgccactagtctctagtcagtgtgttaatcttacaaccagaactcaattaccccctgcatacactaattctttcacacgtggtgtttattaccctgacaaagttttcagatcctcagttttacattcaactcaggacttgttcttacctttcttttccaatgttacttggttccatgctatacatgtctctgggaccaatggtactaagaggtttgataaccctgtcctaccatttaatgatggtgtttattttgcttccactgagaagtctaacataataagaggctggatttttggtactactttagattcgaagacccagtccctacttattgttaataacgctactaatgttgttattaaagtctgtgaatttcaattttgtaatgatccatttttgggtgtttattaccacaaaaacaacaaaagttggatggaaagtgagttcagagtttattctagtgcgaataattgcacttttgaatatgtctctcagccttttcttatggaccttgaaggaaaacagggtaatttcaaaaatcttagggaatttgtgtttaagaatattgatggttattttaaaatatattctaagcacacgcctattaatttagtgcgtgatctccctcagggtttttcggctttagaaccattggtagatttgccaataggtattaacatcactaggtttcaaactttacttgctttacatagaagttatttgactcctggtgattcttcttcaggttggacagctggtgctgcagcttattatgtgggttatcttcaacctaggacttttctattaaaatataatgaaaatggaaccattacagatgctgtagactgtgcacttgaccctctctcagaaacaaagtgtacgttgaaatccttcactgtagaaaaaggaatctatcaaacttctaactttagagtccaaccaacagaatctattgttagatttcctaatattacaaacttgtgcccttttggtgaagtttttaacgccaccagatttgcatctgtttatgcttggaacaggaagagaatcagcaactgtgttgctgattattctgtcctatataattccgcatcattttccacttttaagtgttatggagtgtctcctactaaattaaatgatctctgctttactaatgtctatgcagattcatttgtaattagaggtgatgaagtcagacaaatcgctccagggcaaactggaaagattgctgattataattataaattaccagatgattttacaggctgcgttatagcttggaattctaacaatcttgattctaaggttggtggtaattataattacctgtatagattgtttaggaagtctaatctcaaaccttttgagagagatatttcaactgaaatctatcaggccggtagcacaccttgtaatggtgttgaaggttttaattgttactttcctttacaatcatatggtttccaacccactaatggtgttggttaccaaccatacagagtagtagtactttcttttgaacttctacatgcaccagcaactgtttgtggacctaaaaagtctactaatttggttaaaaacaaatgtgtcaatttcaacttcaatggtttaacaggcacaggtgttcttactgagtctaacaaaaagtttctgcctttccaacaatttggcagagacattgctgacactactgatgctgtccgtgatccacagacacttgagattcttgacattacaccatgttcttttggtggtgtcagtgttataacaccaggaacaaatacttctaaccaggttgctgttctttatcaggatgttaactgcacagaagtccctgttgctattcatgcagatcaacttactcctacttggcgtgtttattctacaggttctaatgtttttcaaacacgtgcaggctgtttaataggggctgaacatgtcaacaactcatatgagtgtgacatacccattggtgcaggtatatgcgctagttatcagactcagactaattctcctcggcgggcacgtagtgtagctagtcaatccatcattgcctacactatgtcacttggtgcagaaaattcagttgcttactctaataactctattgccatacccacaaattttactattagtgttaccacagaaattctaccagtgtctatgaccaagacatcagtagattgtacaatgtacatttgtggtgattcaactgaatgcagcaatcttttgttgcaatatggcagtttttgtacacaattaaaccgtgctttaactggaatagctgttgaacaagacaaaaacacccaagaagtttttgcacaagtcaaacaaatttacaaaacaccaccaattaaagattttggtggttttaatttttcacaaatattaccagatccatcaaaaccaagcaagaggtcatttattgaagatctacttttcaacaaagtgacacttgcagatgctggcttcatcaaacaatatggtgattgccttggtgatattgctgctagagacctcatttgtgcacaaaagtttaacggccttactgttttgccacctttgctcacagatgaaatgattgctcaatacacttctgcactgttagcgggtacaatcacttctggttggacctttggtgcaggtgctgcattacaaataccatttgctatgcaaatggcttataggtttaatggtattggagttacacagaatgttctctatgagaaccaaaaattgattgccaaccaatttaatagtgctattggcaaaattcaagactcactttcttccacagcaagtgcacttggaaaacttcaagatgtggtcaaccaaaatgcacaagctttaaacacgcttgttaaacaacttagctccaattttggtgcaatttcaagtgttttaaatgatatcctttcacgtcttgacaaagttgaggctgaagtgcaaattgataggttgatcacaggcagacttcaaagtttgcagacatatgtgactcaacaattaattagagctgcagaaatcagagcttctgctaatcttgctgctactaaaatgtcagagtgtgtacttggacaatcaaaaagagttgatttttgtggaaagggctatcatcttatgtccttccctcagtcagcacctcatggtgtagtcttcttgcatgtgacttatgtccctgcacaagaaaagaacttcacaactgctcctgccatttgtcatgatggaaaagcacactttcctcgtgaaggtgtctttgtttcaaatggcacacactggtttgtaacacaaaggaatttttatgaaccacaaatcattactacagacaacacatttgtgtctggtaactgtgatgttgtaataggaattgtcaacaacacagtttatgatcctttgcaacctgaattagactcattcaaggaggagttagataaatattttaagaatcatacatcaccagatgttgatttaggtgacatctctggcattaatgcttcagttgtaaacattcaaaaagaaattgaccgcctcaatgaggttgccaagaatttaaatgaatctctcatcgatctccaagaacttggaaagtatgagcagtatataaaatggccatggtacatttggctaggttttatagctggcttgattgccatagtaatggtgacaattatgctttgctgtatgaccagttgctgtagttgtctcaagggctgttgttcttgtggatcctgctgcaaatttgatgaagacgactctgagccagtgctcaaaggagtcaaattacattacacataacgccccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaaacgtctaggccccccgaaccacggggacgtggttttcctttgaaaaacacgatgataatatggccacaaccatggcagattccaacggtactattaccgttgaagagcttaaaaagctccttgaacaatggaacctagtaataggtttcctattccttacatggatttgtcttctacaatttgcctatgccaacaggaataggtttttgtatataattaagttaattttcctctggctgttatggccagtaactttagcttgttttgtgcttgctgctgtttacagaataaattggatcaccggtggaattgctatcgcaatggcttgtcttgtaggcttgatgtggctcagctacttcattgcttctttcagactgtttgcgcgtacgcgttccatgtggtcattcaatccagaaactaacattcttctcaacgtgccactccatggcactattctgaccagaccgcttctagaaagtgaactcgtaatcggagctgtgatccttcgtggacatcttcgtattgctggacaccatctaggacgctgtgacatcaaggacctgcctaaagaaatcactgttgctacatcacgaacgctttcttattacaaattgggagcttcgcagcgtgtagcaggtgactcaggttttgctgcatacagtcgctacaggattggcaactataaattaaacacagaccattccagtagcagtgacaatattgctttgcttgtacagtaacgccccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaaacgtctaggccccccgaaccacggggacgtggttttcctttgaaaaacacgatgataatatggccacaaccatgtctgataatggaccccaaaatcagcgaaatgcaccccgcattacgtttggtggaccctcagattcaactggcagtaaccagaatggagaacgcagtggggcgcgatcaaaacaacgtcggccccaaggtttacccaataatactgcgtcttggttcaccgctctcactcaacatggcaaggaagaccttaaattccctcgaggacaaggcgttccaattaacaccaatagcagtccagatgaccaaattggctactaccgaagagctaccagacgaattcgtggtggtgacggtaaaatgaaagatctcagtccaagatggtatttctactacctaggaactgggccagaagctggacttccctatggtgctaacaaagacggcatcatatgggttgcaactgagggagccttgaatacaccaaaagatcacattggcacccgcaatcctgctaacaatgctgcaatcgtgctacaacttcctcaaggaacaacattgccaaaaggcttctacgcagaagggagcagaggcggcagtcaagcctcttctcgttcctcatcacgtagtcgcaacagttcaagaaattcaactccaggcagcagtaggggaacttctcctgctagaatggctggcaatggcggtgatgctgctcttgctttgctgctgcttgacagattgaaccagcttgagagcaaaatgtctggtaaaggccaacaacaacaaggccaaactgtcactaagaaatctgctgctgaggcttctaagaagcctcggcaaaaacgtactgccactaaagcatacaatgtaacacaagctttcggcagacgtggtccagaacaaacccaaggaaattttggggaccaggaactaatcagacaaggaactgattacaaacattggccgcaaattgcacaatttgcccccagcgcttcagcgttcttcggaatgtcgcgcattggcatggaagtcacaccttcgggaacgtggttgacctacacaggtgccatcaaattggatgacaaagatccaaatttcaaagatcaagtcattttgctgaataagcatattgacgcatacaaaacattcccaccaacagagcctaaaaaggacaaaaagaagaaggctgatgaaactcaagccttaccgcagagacagaagaaacagcaaactgtgactcttcttcctgctgcagatttggatgatttctccaaacaattgcaacaatccatgagcagtgctgactcaactcaggcctaatcgatccctaccggttagtaatgagtttaaacgggggaggctaactgaaacacggaaggagacaataccggaaggaacccgcgctatgacggcaataaaaagacagaataaaacgcacgggtgttgggtcgtttgttcataaacgcggggttcggtcccagggctggcactctgtcgataccccaccgagaccccattggggccaatacgcccgcgtttcttccttttccccaccccaccccccaagttcgggtgaaggcccagggctcgcagccaacgtcggggcggcaggccctgccatagctttttggggcggcttttctcgagtccttccttaaggacgtgctcgtcaatttttgttttgagagtcatctattcggatgcttttcatgaagttttttatgaccctgaccggcaccctgcgcaaggccttcgccaccaccctggccgccgccatgctgatcggcaccctggccggctgctcctccgccgcatacaacaagtctgacctcgtttcgaagatcgcccagaagtccaacctgaccaaggctcaggccgaggctgctgttaacgccttccaggatgtgttcgtcgaggctatgaagtccggcgaaggcctgaagctcaccggcctgttctccgctgagcgcgtcaagcgcccggctcgcaccggccgcaacccgcgcactggcgagcagattgacattccggcttcctacggcgttcgtatctccgctggctccctgctgaagaaggccgtcaccgagtatggacggaagaagcgcaggcagcgacggcgatgagggtttgcgcttgcgtcgtggagggagcggaacgccgaaaaaggatccacgcgctggagatgttcaacgagtagatcgccacggcgacctccttccacgcgtgcgggcacggggattctcaaggggccggcccgaggccccttgagcccgccgggaggcgcccccggcagggcgggaatccaaagggcggagccctgtggccctccccgggcaggggcgggatcgtcaagggcggagcccttggccccctcgggagagcgcactgacacaatgctacctccggtagcattaagtgcgccctccgccatgcggagggacgggccgcgaccggatatgcggggaacgtccacgacgcgtcttccgtgtcctccgtcctgccttgtgccgtcgattataatcctcggataacggacgatgattgaaccgattggaggaaacgagatgccgaagagtttcgcgcagcagatcgaggacgacgagaacaagatcaagcgcatccgggagcaccagcgcatggtcagggccaaacaagccaaacaggagcgcaacgcccgcaccaaaaggctcgtggagaccggagcgatagtggaaaaagcgcacggcggcgcgtacgacgacgaaggacggcagacgttctcggatggtctgaacggcatcatctcggtctacgacccgtcccgcggcggcaacgtggacatgagagtcatcgacgtaatcgaccggcgcatccccagattgccaaggtccgaaaccacaacaggcacggcagcggcggcatcgcgaaccgtgcaagccaccgcaccacagccagcccacgcccaaccgcaaagcttcacgcccaacccccagcgcatcgaacaccggacaggagcacagcagccggatcggtgggcctagccgggacggcgcagccctcgctccgctgggctgaaatatctgacttggttcgtaacatatttccgacatgtcggaaatatgtcgtatcatcgttgctatgagtactgaacttcgtgagcagtgggagcagctttacctgccgctgcgcccgctgtgcacgaacgacttcatcgagggcgtgtaccggcagcccagggcgaaggcgctggagggctaccggtacatcgaggcgaaccccaagaccgtcagcgacctgctcgtggtcgacatcgacgacgcgaacgcccgcgcgatggccctatgggagcatgaggggatgctgcccaacgtgatcgtggagaacccgagaaacggccacgcgcacgccgtgtgggcgctggccgccccgttcagtcgcaccgaatacgcgcggcgcaagccgctcgccctggccgcggcggtcacggaggggctgcgccgctcgtgcgacggggacaagggatattcggggctgatgaccaagaacccgctgcacgaggcgtggaacagcgaactggtcaccgatcacctgtacgggctggacgaactgcgcgaggcgctggaggagtccggggacatgccgccggcctcgtggaagcgcacgaaacggcgcaacgtggtcggattgggccgcaactgccatctgttcgagacggcgcgcacatgggcgtacagggaggtgcgccaccactggggagacagcgaggggctgcggctggccatctcggccgaggcgcacgaaatgaacgccgaactgttccccgagccactggcatggtcggaggtcgagcagatcgccaagagcatccacaagtggatcgtcacccaaagccgcatgtggcgcgacggccccaccgtctacgaggcgacattcatcaccgtccaaagcgcccgagggaaaaagtcaggcaaggccagacgagaaacattcgaactattcgcgagcgaggagatgacgtaa

SEQ ID NO: 46 - protein, 1273 aa, SARS-CoV-2
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 47 - protein, 222 aa, SARS-CoV-2
MADSNGTITVEELKKLLEQWNLVIGFLFLTWICLLQFAYANRNRFLYIIKLIFLWLLWPVTLACFVLAAVYRINWITGGIAIAMACLVGLMWLSYFIASFRLFARTRSMWSFNPETNILLNVPLHGTILTRPLLESELVIGAVILRGHLRIAGHHLGRCDIKDLPKEITVATSRTLSYYKLGASQRVAGDSGFAAYSRYRIGNYKLNTDHSSSSDNIALLVQ

SEQ ID NO: 48 - protein, 419 aa, SARS-CoV-2
MSDNGPQNQRNAPRITFGGPSDSTGSNQNGERSGARSKQRRPQGLPNNTASWFTALTQHGKEDLKFPRGQGVPINTNSSPDDQIGYYRRATRRIRGGDGKMKDLSPRWYFYYLGTGPEAGLPYGANKDGIIWVATEGALNTPKDHIGTRNPANNAAIVLQLPQGTTLPKGFYAEGSRGGSQASSRSSSRSRNSSRNSTPGSSRGTSPARMAGNGGDAALALLLLDRLNQLESKMSGKGQQQQGQTVTKKSAAEASKKPRQKRTATKAYNVTQAFGRRGPEQTQGNFGDQELIRQGTDYKHWPQIAQFAPSASAFFGMSRIGMEVTPSGTWLTYTGAIKLDDKDPNFKDQVILLNKHIDAYKTFPPTEPKKDKKKKADETQALPQRQKKQQTVTLLPAADLDDFSKQLQQSMSSADSTQA

SEQ ID NO: 49 - DNA, 8978 bp, Artificial Sequence; identical to SEQ ID NO: 44
  DNA sequence of pFRG-CMV-Sgene

SEQ ID NO: 50 - protein, 223 aa, SARS-CoV-2
RVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNF

SEQ ID NO: 51 - protein, 72 aa, SARS-CoV-2
NSNNLDSKVGGNYNYLYRLFRKSNLKPFERDISTEIYQAGSTPCNGVEGFNCYFPLQSYGFQPTNGVGYQPY

SEQ ID NO: 52 - protein, 1261 aa, SARS-Cov-2
SQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 53 - protein, 1270 aa, SARS-CoV-2
MFVFLVLLPLVSSQCVNFTTRTQLPPAYTNSFTRGVYYPDKVFRSSVLHSTQDLFLPFFSNVTWFHAIHVSGTNGTKRFANPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRGLPQGFSALEPLVDLPIGINITRFQTLHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGNIADYNYKLPDDFTGCVIAWNSNNLDSKVGGNYNYLYRLFRKSNLKPFERDISTEIYQAGSTPCNGVKGFNCYFPLQSYGFQPTYGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEHVNNSYECDIPIGAGICASYQTQTNSPRRARSVASQSIIAYTMSLGVENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 54 - protein, 223 aa, SARS-CoV-2
RVQPTESIVRFPNITNLCPFGEVFNATRFASVYAWNRKRISNCVADYSVLYNSASFSTFKCYGVSPTKLNDLCFTNVYADSFVIRGDEVRQIAPGQTGNIADYNYKLPDDFTGCVIAWNSNNLDSKVGGNYNYLYRLFRKSNLKPFERDISTEIYQAGSTPCNGVKGFNCYFPLQSYGFQPTYGVGYQPYRVVVLSFELLHAPATVCGPKKSTNLVKNKCVNF

SEQ ID NO: 55 - protein, 71 aa, SARS-CoV-2
SNNLDSKVGGNYNYLYRLFRKSNLKPFERDISTEIYQAGSTPCNGVKGFNCYFPLQSYGFQPTYGVGYQPY

SEQ ID NO: 56 - protein, 1258 aa, SARS-CoV-2
SQCVNFTTRTQLPPAYTNSFTRGVYYPDKVFRSSVLHSTQDLFLPFFSNVTWFHAIHVSGTNGTKRFANPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRGLPQGFSALEPLVDLPIGINITRFQTLHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGNIADYNYKLPDDFTGCVIAWNSNNLDSKVGGNYNYLYRLFRKSNLKPFERDISTEIYQAGSTPCNGVKGFNCYFPLQSYGFQPTYGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEHVNNSYECDIPIGAGICASYQTQTNSPRRARSVASQSIIAYTMSLGVENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT